SEQUENCE LISTING (38 sequences)

SEQ ID NO: 1 - DNA, 388 bp, Artificial Sequence
  16S r
ctgccctttgcacagggatagccattggaaacgatgattaaaacctgataacaccatttggttacatgagcagatggtcaaagatttatcggcaaaggatgggcctgcgtctgattagctagttggtaaggtaacggcttaccaaggcgacgatcagtagccgacctgagagggtgaacggccacattggaactgagacacggtccaaactcctacgggaggcagcagtggggaatattgcacaatgggcgaaagcctgatgcagcaacgccgcgtgaaggaagaaggccttcgggtcgtaaacttctgtccttggggaagaagaactgacggtacccaaggaggaagccccggctaactacgtgccagcagccgcggtaatacgtag

SEQ ID NO: 2 - DNA, 378 bp, Artificial Sequence
  16S r
cagttagaaatgactgctaataccgcataagaccacaaagccgcatggctrwgtggtaaaaactccggtggtgtaagatgggcccgcgtctgattaggtagttggcggggtaacggcccaccaagccgacgatcagtagccgacctgagagggtgaccggccacattgggactgagacacggcccagactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagccccggctaactacgtgccagcagccgcggtaatacgtagggggcaagcgtta

SEQ ID NO: 3 - DNA, 133 bp, Artificial Sequence
  16S r
agttggaaacgactgctaataccgcatgatacatttgggtcgcatggtctgaatgtcaaagatttatcgccgaaagatggcctcgcgtctgattagctagttggtggggtaacggcccaccaaggcgacgatc

SEQ ID NO: 4 - DNA, 200 bp, Artificial Sequence
  16S r
tacagggggataacacttagaaataggtgctaataccgcataagcgcacaggggcgcatgccccggtgtgaaaaactccggtggtatatgatggacccgcgtctgattagccagttggcagggtaacggcctaccaaagcgacratcartagccggcctgagagggcggacggccacattgggactgagacacggcccaa

SEQ ID NO: 5 - DNA, 394 bp, Artificial Sequence
  16S r
cagggatagccattggaaacgatgattaaaacctgataacaccatttggttacatgagcagatggtcaaagatttatcggcaaaggatgggcctgcgtctgattagctagttggtaaggtaacggcttaccaaggcgacgatcagtagccgacctgagagggtgaacggccacattggaactgagacacggtccaaactcctacgggaggcagcagtggggaatattgcacaatgggcgaaagcctgatgcagcaacgccgcgtgaaggaagaaggccttcgggtcgtaaacttctgtccttggggaagaagaactgacggtacccaaggaggaagccccggctaactacgtgccagcagccgcggtaatacgtagggggcaagcgttatccgg

SEQ ID NO: 6 - DNA, 200 bp, Artificial Sequence; identical to SEQ ID NO: 4
  16S r

SEQ ID NO: 7 - DNA, 279 bp, Artificial Sequence
  16S r
cattggaaacgatgattaaaacctgataacaccatttggttacatgagcagatggtcaaagatttatcggcaaaggatgggcctgcgtctgattagctagttggtaaggtaacggcttaccaaggcgacgatcagtagccgacctgagagggtgaacggccacattggaactgagacacggtccaaactcctacgggaggcagcagtggggaatattgcacaatgggcgaaagcctgatgcagcaacsccgcgtgaaggaagaaggccttcgggtcgta

SEQ ID NO: 8 - DNA, 108 bp, Artificial Sequence
  Synthetic construct
gggggacaacagttagaaatgactgctaataccgcataagcgcacgggaacgcatgtttctgtgtgaaaaactccggtggtgtaagatgggcccgcgttggattaggt

SEQ ID NO: 9 - DNA, 149 bp, Artificial Sequence
  16S r
ccattggaaacgatgattaaaacctgataacaccatttggttacatgagcagatggtcaaagatttatcggcaaaggatgggcctgcgtctgattagctakttggtaaggtaacggcttaccaaggcgacgatcagtagcckacctgag

SEQ ID NO: 10 - DNA, 214 bp, Artificial Sequence
  16S r
taataccgcatgagactacagtactacatggtacagtggccaaaggagcaatccgctgaaagatgggctcgcgtccgattagatagttggcggggtaacggcccaccaagtcgacgatcggtagccggactgagaggttgaacggccacrttgggactgagacacggcccagactcctacgggaggcagcagtgagggatattggtcaatgggg

SEQ ID NO: 11 - DNA, 291 bp, Artificial Sequence
  16S r
agccattggaaacgatgattaaaacctgataacaccatttggttacatgagcagatggtcaaagatttatcggcaaaggatgggcctgcgtctgattagctagttggtaaggtaacggcttaccaaggcgacgatcagtagccgacctgagagggtgaacggccacattggaactgagacacggtccaaactcctacgggaggcagcagtggggaatattgcacaatgggcgaaagcctgatgcagcaacgccgcgtgaaggaagaaggccttcgggtcgtaaacttctgt

SEQ ID NO: 12 - DNA, 405 bp, Artificial Sequence
  16S r
gtaggcaacctgcccttwgcacagggatagccattggaaacgatgattaaaacctgataacaccatttggttacatgagcagatggtcaaagatttatcggcaaaggatgggcctgcgtctgattagctagttggtaaggtaacggcttaccaaggcgacgatcagtagccgacctgagagggtgaacggccacattggaactgagacacggtccaaactcctacgggaggcagcagtggggaatattgcacaatgggcgaaagcctgatgcagcaacgccgcgtgaaggaagaaggccttcgggtcgtaaacttctgtccttggggaagaagaactgacggtacccaaggaggaagccccggctaactacgtgccagcagccgcggtaatacgtagggggcaag

SEQ ID NO: 13 - DNA, 171 bp, Artificial Sequence
  16S r
gctaataccgcataagagtagatgttgcatgacatttgcttaaaaggtgcaattgcatcactaccagatggacctgcgttgtattagctagttggtggggtaacggctcaccaaggcgacgatacatagccgacctgagagggtgatcggccacactgggactgagacacg

SEQ ID NO: 14 - DNA, 164 bp, Artificial Sequence
  16S r
ataccgcataagagtagatgttgcatgacatttgcttaaaaggtgcaattgcatcactaccagatggacctgcgttgtattagctagttggtggggtaacggctcaccaasgcgacgatacatagccgacctgagagggtgatcggccrcactgggaccgagac

SEQ ID NO: 15 - DNA, 142 bp, Artificial Sequence
  16S r
ccccatagagggggacaacagctggaaacggctgctaataccgcatagcaggaaagagacgcatgtctttttcttcaaagatttatcgctatgggatggacccgcgtctgattagctagttggtaaggtaacggcctaccaa

SEQ ID NO: 16 - DNA, 252 bp, Artificial Sequence
  16S r
aactcctacgggaggcagcagtggggaatattgcacaatgggcgcaagcctgatgcagcgacsccgcgtgagcgaagaagtatttcggtatgtaaagctctatcagcagggaagaaaatgacrgtacctgactaaaaagctccggctaaatacrtgycagcasccscggtaatacgtatggagcaagcgttatccggaattactgkgtgtaaagggagcgtatacggatgtgcaagtctgatgtgaaaggcg

SEQ ID NO: 17 - DNA, 484 bp, Artificial Sequence
  16S r
ggggcaagcgttatccggaattattgggcgtaaaagagtacgtaggtggcaacctaagcgcagggtttaaggcaatggctcaaccattgttcgcccctgcgaactggagaatgcttgagtgcaggagaggaaaagcggaattcctagtgtagcggtgaaaatgcgtagatattaggaggaacaccagtggcgaaggcggctttctggactgtaactgacactgaggtacgaaagcgtggggagcaaacaggattagataccctggtagtccacgccgtaaacgatgagcactaggtgtcggggtcgcaagacttcggtgccgtagttaacgcattaagtgctccgcctgggggagtacgcacgcaagtgtgaaactcaaaggaaattgacgggggaccccgcacaagcagcggagcatgtggtttaattcgaagcaacgcgaaagaaaccttaccaggacttgacatccctctgacagaccctt

SEQ ID NO: 18 - DNA, 729 bp, Artificial Sequence
  16S r
ctgatgcagcgacgccgcgtgagcgaagaagtatttcggtatgtaaagctctatcagcagggaagaaaatgacggtacctgagtaagaagctccggctaaatacgtgccagcagccgcggtaatacgtatggagcaagcgttatccggatttactgggtgtaaagggagcgcaggcggcagggcaagtctgatgtgaaataccggggctcaaccccggagctgcattggaaactgttctgctggagtgtcggagaggcaggcggaattcctagtgtagcggtgaaatgcgtagatattaggaggaacaccagtggcgaaggcggcctgctggacgataactgacgctgaggctcgaaagcgtggggagcaaacaggattagataccctggtagtccacgccgtaaacgatgaatactaggtgtcggggagcaaagctcttcggtgccgcagcaaacgcagtaagtattccacctggggagtacgttcgcaagaatgaaactcaaaggaattgacggggacccgcacaagcggtggagcatgtggtttaattcgaagcaacgcgaagaaccttaccaagccttgacatcccgatgacagcatatgtaatgtatgttccctttttgggcattggagacaggtggtgcatggttgtcgtcagctcgtgtcgtgagatgttgggttaagtcccgcaacgagcgcaacccttatccttagtagccagcaggcag

SEQ ID NO: 19 - DNA, 448 bp, Artificial Sequence
  16S r
atgtaaagctctatcagcagggaagaaaatgacggtacctgactaagaagccccggctaactacgtgccagcagccgcggtaatacgtagggggcaagcgttatccggatttactgggtgtaaagggagcgtagacggcagcgcaagtctgaagtgaaatcccatggcttaaccatggaactgctttggaaactgtgcagctggagtgcaggagaggtaagcggaattcctagtgtagcggtgaaatgcgtakatattaggaggaacaccagtggcgaaggcggcttactggactgtaactgacgttgaggctcgaaagcgtggggagcaaacaggattagataccctggtagtccacgccstaaacgatgattactaggtgttgggggaccaaggtccttcggtgccggcgcaaacgcattaagtaatccacctggggagtacgttc

SEQ ID NO: 20 - DNA, 659 bp, Artificial Sequence
  16S r
taactacgtgccagcagccgcggttaatacgtagggggggcaagcgttatccggaattattgggcgtaaagagtacgtaggtggcaaccctaagcgcaggggttttaaggcaatggctcaaccattgttcgccctgcgactgggatgcttgagtgcaggagaggaaaagcggaattcctagtgtagcggtgaaaatgcgtagatattaggaggaacaccagtggcgaaggcggctttctggactgtaactgacactgaggtacgaaagcgtggggagcaaacaggattagataccctggtagtccacgccgtaaacgatgagcactaggtgtcggggtcgcaagacttcggtgccgtagttaacgcattaagtgctccgcctggggagtacgcacgcaagtgtgaaactcaaaggaattgacggggacccgcacaagcagcggagcatgtggtttaattcgaagcaacgcgaagaaccttaccaggacttgacatccctctgacagacccttaatcgggtttttctacggacagaggaacaggtggtgcatgggttgtcgtcagctcgtgtcgtgagatgttgggttaagtcccgcaacgagcgcaactctttgccattagtttgccagcagtaagatgggcactctagtgggactgcc

SEQ ID NO: 21 - DNA, 398 bp, Artificial Sequence
  16S r
aaatgcgtagatattaggaggaacaccagtggcgaaggcggctttctggactgtaactgacactgaggtacgaaagcgtgggggagcaaacaggattagataccctggtagtccacgccgtaaacgatgagcactaggtgtcggggtcgcaagacttcggtgccgtagttaacgcattaagtgctccgcctgggggagtacgcacgcaagtgtgaaactcaaaggaattgacggggacccgcacaagcagcggagcatgtggtttaattcgaagcaacgcgaagaaaccttaccaggacttgacatccctctgacagacccttaatcgggtttttttctacggacagaggagacaggtggtgcatggttgtcgtcagctcgtgtcgtgagatgttggg

SEQ ID NO: 22 - DNA, 418 bp, Artificial Sequence
  16S r
acgtagggggcaagcgttatcccggaattattgggcgtaaagagtacgtaggtggcaacctaagcgcaggggtttaaggcaatggctcaaccattgttcgccctgcgaactgggatgcttgagtgcaggagaggaaagcggaattcctagtgtagcggtgaaatgcgtagatattaggaggaacaccagtggcgaaggcggcttttctggactgtaactgacactgaggtacgaaaagcgtgggggagcaaacaggattagatacccctggtagtccacgccgtaaacgatgagcactaggtgtcgggggtcgcaagacttcggtgccgtagttaacgcattaagtgcctccgcctgggggagtacgcacgccaagtgtgaaactcataggaattgacggggacccgcacaagcagcg

SEQ ID NO: 23 - DNA, 899 bp, Artificial Sequence
  16S r
gagacacggtccaaactcctacgggaggcagcagtggggaatattgcacaatgggcgaaagcctgatgcagcaacgccgcgtgaaggaagaaggccttcgggtcgtaaacttctgtccttggggaagaagaactgacggtacccaaggaggaagccccggctaactacgtgccagcagccgcggtaatacgtagggggcaagcgttatccggaattattgggcgtaaagagtacgtaggtggcaacctaagcgcagggtttaaggcaatggctcaaccattgttcgccctgcgaactgggatgcttgagtgcaggagaggaaagcggaattcctagtgtagcggtgaaatgcgtagatattaggaggaacaccagtggcgaaggcggctttctggactgtaactgacactgaggtacgaaagcgtggggagcaaacaggattagataccctggtagtccacgccgtaaacgatgagcactaggtgtcggggtcgcaagacttcggtgccgtagttaacgcattaagtgctccgcctggggagtacgcacgcaagtgtgaaactcaaaggaattgacggggacccgcacaagcagcggagcatgtggtttaattcgaagcaacgcgaagaaccttaccaggacttgacatccctctgacagacccttaatcgggtttttctacggacagaggagacaggtggtgcatggttgtcgtcagctcgtgtcgtgagatgttgggttaagtcccgcaacgagcgcaacccttgccattagttgccagcagtaagatgggcactctagtgggactgccggggacaactcggaggaaggtggggatgacgtcaaatcatcatgccccttatgttctgggctacacacgtgctacaatggccggtaca

SEQ ID NO: 24 - DNA, 679 bp, Artificial Sequence
  16S r
gagcgagaagctgatgacagatacttcggttgaaggagtcagtggaaagcggcggacgggtgagtaacgcgtaggcaacctgccctttgcacagggatagccattggaaacgatgattaaaacctgataacaccatttggttacatgagcagatggtcaaagatttatcggcaaaggatgggcctgcgtctgattagctagttggtaaggtaacggcttaccaaggcgacgatcagtagccgacctgagagggtgaacggccacattggaactgagacacggtccaaactcctacgggaggcagcagtggggaatattgcacaatgggcgaaagcctgatgcagcaacgccgcgtgaaggaagaaggccttcgggtcgtaaacttctgtccttggggaagaagaactgacggtacccaaggaggaagccccggctaactacgtgccagcagccgcggtaatacgtagggggcaagcgttatccggaattattgggcgtaaagagtacgtaggtggcaacctaagcgcagggtttaaggcaatggctcaaccattgttcgccctgcgaactgggatgcttgagtgcaggagaggaaagcggaattcctagtgtagcggtgaaatgcgtagatattaggaggaacaccagtggcgaaggcggctttctggactgtaactga

SEQ ID NO: 25 - DNA, 583 bp, Artificial Sequence
  16S r
agcgagaagctgatgacagatacttcggttgaaggagtcagtggaaagcggcggacgggtgagtaacgcgtaggcaacctgccctttgcacagggatagccattggaaacgatgattaaaacctgataacaccatttggttacatgagcagatggtcaaagatttatcggcaaaggatgggcctgcgtctgattagctagttggtaaggtaacggcttaccaaggcgacgatcagtagccgacctgagagggtgaacggccacattggaactgagacacggtccaaactcctacgggaggcagcagtggggaatattgcacaatgggcgaaagcctgatgcagcaacgccgcgtgaaggaagaaggccttcgggtcgtaaacttctgtccttggggaagaagaactgacggtacccaaggaggaagccccggctaactacgtgccagcagccgcggtaatacgtagggggcaagcgttatccggaattattgggcgtaaagagtacgtaggtggcaacctaagcgcagggtttaaggcaatggctcaaccattgttcgccctgcgaactgggatgcttgagtg

SEQ ID NO: 26 - DNA, 652 bp, Artificial Sequence
  16S r
tcgaacgaagcgatttaacggaagttttcggatggaagttgaattgactgagtggcggacgggtgagtaacgcgtgggtaacctgccttgtactgggggacaacagttagaaatgactgctaataccgcataagcgcacagtatcgcatgatacagtgtgaaaaactccggtggtacaagatggacccgcgtctgattagctagttggtaaggtaacggcttaccaaggcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggtaaagcaagtctgaagtgaaagcccgcggctcaactgcgggactgctttggaaactgtttaactggagtgtcggagaggtaagtggaattcctagtgtagcggtgaaatgcgtagatattaggaggaacaccagtg

SEQ ID NO: 27 - DNA, 611 bp, Artificial Sequence
  16S r
aagtgcgtgagagtaactgttcacgtttcgacggtatctaaccagaaagccacggctaactacgtgccagcagccgcggtaatacgtaggtggcaagcgttatccggatttattgggcgtaaagggaacgcaggcggtcttttaagtctgatgtgaaagccttcggcttaaccggagtagtgcattggaaactgggagacttgagtgcagaagaggagagtggaactccatgtgtagcggtgaaatgcgtagatatatggaagaacaccagtggcgaaagcggctctctggtctgtaactgacgctgaggttcgaaagcgtgggtagcaaacaggattagataccctggtagtcccgccgtaaacgatgaatgctaagtgttggagggtttccgcccttcagtgctgcagctaacgcaataagcattccgcctggggagtacgaccgcaaggttgaaactcaaaggaattgacggggggcccgcacaagcggtggagcatgtggtttaattcgaagcaacgcgaagaacccttaccaaggtcttgacatctttttgacaatccctagagataggactttcccttcggggacaaaatgacaggtggtgcatg

SEQ ID NO: 28 - DNA, 666 bp, Artificial Sequence
  16S r
gactcctacgggaggcagcagtgagggatattggtcaatgggggaaaccctgaaccagcaacgccgcgtgagggaagacggttttcggattgtaaacctctgtcctctgtgaagataatgacggtagcagaggaggaagctccggctaactacgtgccagcagccgcggtaatacgtagggagcaagcgttgtccggatttactgggtgtaaagggtgcgtaggcggccttgcaagtcagaagtgaaatccatgggcttaacccgtgaactgcttttgaaactgtagggcttgagtgaagtagaggcaggcggaattcccggtgtagcggtgaaatgcgtagagatcgggaggaacaccagtggcgaaggcggcctgctgggctttaactgacgctgaagcacgaaagcgtgggtagcaaacaggattagataccctggtagtccacgccgtaaacgatgattactaggtgtggggggggtctgacccccctccgtgccggagttaacacaataagtaatccacctgggggagtacggccgcaaggctgaaactcaaaggaaattgacggggggcccgcacaagcagtggagtatgtggattaattcgaagccaacgcgaagaaccttaccaggtcttgacatccccggcgaccggcttagagata

SEQ ID NO: 29 - DNA, 740 bp, Artificial Sequence
  16S r
cacggcccagactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcggtgcaagccagatgtgaaagcccggggctcaaccccgggactgcatttggaactgtgctgctagagtgtcggagaggcaggcggaattcctagtgtagcggtgaaatgcgtagatattaggaggaacaccagtggcgaaggcggcctgctggagatgactgacgttgaggctcgaaagcgtggggagcaaacaggattagataccctggtagtccacgccgtaaacgatgactactaggtgtcgggcagcaaagctgttcggtgccgcagccaacgcaataagtagtccacctggggagtacgttcgcaagaatgaaactcaaaggaattgacggggacccgcacaagcggtggagcatgtggtttaattcgaagcaacgcgaagaaccttacctggycttgacatcccccctgaccggctcgtaatggggcctttccttcgggacaagggggagaacaggtggtgcatggattgtcgtcagctcgtgtcgtgagatgttgg

SEQ ID NO: 30 - DNA, 259 bp, Artificial Sequence
  16S r
aagtcgaacgggaagcggggcctccgggccccgccgagagtggcgaacggctgagtaacacgtgggcaacctgccccctccaccgggacagcctcgggaaaccgtgggtaataccggatactccgggacggccgcatggccggcccgggaaagcccagacgggaggggatgggcccgcggcctgttagctagtcggcggggtaacggcccaccgaggcgattatgggtagccgggttgagagaccgaccagccagattg

SEQ ID NO: 31 - DNA, 347 bp, Artificial Sequence
  16S r
aaatgcgtagatatcaggaggaacaccagtggcgaaggcggcctgctggacgatgactgacgctgaggctcgaaagcgtggggagcaaacaggattagataccctggtagtccacgccgtaaacgatgaataccaggtgtcggggagcagggctcttcggtgccgcagcaaacgcagtaagtattccacctggggagtacgttcgcaagaatgaaactcaaarggaattgacggggacccgcacaagcggtggagcatgtggtttaattcgaagcaacgcgaagacccttactcaggccttgacatcccgggtgacagcatatgtaatgtatgttcccttcggggca

SEQ ID NO: 32 - DNA, 634 bp, Artificial Sequence
  16S r
gctcaccaaggcgacgatcagtagccggcctgagagggtgaacggccacattgggactgagacacggccgaaactcctacgggaggcagcagtggggaatattgsasaatgggggaaaccctgatgcagcgacgccgcgtgaaggaagaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtaggcggttcagcaagtcagaagtgaaagcccggggctcaactccgggactgcttttgaaactgttgaactagattgcaggagaggtaagtggaattcctagtgtagcggtgaaatgcgtagatattaggaggaacaccagtggcgaaagcggcttactggactgtaaatgacgctgaggctcgaaagcgtgrgggagcaaacaggattagataccctggtagtccacgccgtaaacgatgaatactaggtgtcaggcgccataggcgtttggtgccgcagcaaacgcaataagtattccacctggaggaagtacgttcgcaagaatgaaactcaaaggaatt

SEQ ID NO: 33 - DNA, 19 bp, Artificial Sequence
  Synthetic construct
aagcgaaactggcggaaac

SEQ ID NO: 34 - DNA, 20 bp, Artificial Sequence
  Synthetic construct
taaccgatgttgggcatcag

SEQ ID NO: 35 - DNA, 24 bp, Artificial Sequence
  Synthetic construct
tcctctgacatttgcaggtctatc

SEQ ID NO: 36 - DNA, 20 bp, Artificial Sequence
  Synthetic construct
aaaggcattggctggaagaa

SEQ ID NO: 37 - DNA, 21 bp, Artificial Sequence
  Synthetic construct
ggaggtggtgatagccggtat

SEQ ID NO: 38 - DNA, 21 bp, Artificial Sequence
  Synthetic construct
tgggtaatccatagagcccag

(19) United States
(12) Patent Application Publication
(10) Pub. No.: US 2022/0265734 A1
(43) Pub. Date: Aug. 25, 2022

(54) CLOSTRIDIA CONSORTIA COMPOSITIONS AND METHODS OF TREATING OBESITY, METABOLIC SYNDROME AND IRRITABLE BOWEL DISEASE

(71) Applicant: University of Utah Research Foundation, Salt Lake City, UT (US)

(72) Inventor: June L. Round, Salt Lake City, UT (US)

(21) Appl. No.: 17/625,866

(22) PCT Filed: Jul. 17, 2020
(86) PCT No.: PCT/US2020/042578

§ 371 Date: Jan. 10, 2022

Related U.S. Application Data
(60) Provisional application No. 62/875,194, filed on Jul. 17, 2019.

(51) Int. Cl. A61K 35/742; C12N 1/20; A61P 3/04; A61P 3/10; A61P 1/00

(52) U.S. Cl. CPC A61K 35/742; C12N 1/20; C12R 2001/145; A61P 3/10; A61P 1/00; A61P 3/04

(57) ABSTRACT

Disclosed herein, are compositions comprising a consortium of Clostridia, and methods of treating obesity, metabolic syndrome, irritable bowel disease, as well as reducing weight gain and inhibiting lipid absorption in the small intestine by administering the compositions to a subject.

### CROSS REFERENCE TO RELATED APPLICATIONS

This application claims the benefit of the filing dates of U.S. Provisional Application No. 62/875,194, filed on Jul. 17, 2019. The content of this earlier filed application is hereby incorporated by reference in its entirety.

### REFERENCE TO A SEQUENCE LISTING

The Sequence Listing submitted herein as a text file named “21101_0401P1_SL.txt,” created on Jul. 16, 2020, and having a size of 24,576 bytes is hereby incorporated by reference pursuant to 37 C.F.R. § 1.52(e)(5).

### BACKGROUND

The microbiota influences host metabolism and obesity, yet organisms that protect from disease remain unknown.

### SUMMARY

Disclosed herein are consortia of bacteria. Disclosed herein are Clostridium consortia.

Disclosed herein are compositions comprising a supernatant from a Clostridia consortium.

Disclosed herein are compositions comprising a Clostridium consortium.

Disclosed herein are a consortium of bacteria comprising Clostridia anaerovorax strain, Clostridium XIVa, Clostridium IV, and Lachnospiraceae spps, wherein the consortium suppresses expression of lipid adsorption genes within intestinal epithelia in a subject compared to a subject where the consortium has not been administered.

Disclosed herein are methods of altering relative abundance of microbiota in a subject, the methods comprising administering to the subject an effective dose of any of the compositions described herein, thereby altering the relative abundance of microbiota in the subject.

Disclosed herein are methods of treating a subject with obesity. In some aspects, the methods can comprise administering to the subject any of the compositions disclosed herein, wherein the relative abundance of Clostridia is increased in the subject compared to the relative abundance prior to administration.

Disclosed herein are methods of treating a subject with metabolic syndrome. In some aspects, the methods can comprise administering to the subject any of the compositions disclosed herein, wherein the relative abundance of Clostridia is increased in the subject compared to the relative abundance prior to administration.

Disclosed herein are methods of treating a subject with irritable bowel disease. In some aspects, the methods can comprise administering to the subject any of the compositions disclosed herein, wherein the relative abundance of Clostridia is increased in the subject compared to the relative abundance prior to administration.

Disclosed herein are methods of reducing weight gain in a subject. In some aspects, the methods can comprise administering to the subject any of the compositions disclosed herein, wherein the relative abundance of Clostridia is increased in the subject compared to the relative abundance prior to administration.

Disclosed herein are methods of inhibiting lipid absorption in a subject's small intestine. In some aspects, the methods can comprise administering to the subject any of the compositions disclosed herein, wherein the relative abundance of Clostridia is increased in the subject compared to the relative abundance prior to administration.

Disclosed herein are methods of downregulating CD36 in a subject's liver. In some aspects, the methods can comprise administering to the subject any of the compositions disclosed herein, wherein the relative abundance of Clostridia is increased in the subject compared to the relative abundance prior to administration.

### BRIEF DESCRIPTION OF THE DRAWINGS

The accompanying figures, which are incorporated in and constitute a part of this specification, illustrate several aspects and together with the description serve to explain the principles of the invention.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

FIGS. 1A-J shows defective T cell signaling in the gut leads to age-associated obesity. FIG. 1A shows a representative image of 6-month WT and T-Myd88^{-/-} mice. FIG. 1B shows the percentage of weight gained as mice age, starting at 2 months of age (WT, n=8; T-Myd88^{-/-}, n=7 plotted). Representative of three independent experiments. FIG. 1C shows the fat accumulation as mice age, starting at 2 months of age (WT, n=8; T-Myd88^{-/-}, n=7 plotted.) Representative of three independent experiments. FIG. 1D shows the total weight of 1-year-old WT and T-Myd88^{-/-} mice (n=6). Representative of three independent experiments. FIG. 1E shows the total fat percentage as measured by NMR of 1-year-old WT and T-Myd88^{-/-} mice (n=6). Representative of three independent experiments. FIG. 1F shows the fasting serum insulin concentrations from 1-year-old WT and T-Myd88^{-/-} mice (WT, n=9; T-Myd88^{-/-}, n=10). Data pooled from three independent experiments. FIG. 1G shows the homeostatic model assessment (HOMA-IR) of 1-year-old WT and T-Myd88^{-/-} mice. (WT, n=9; T-Myd88^{-/-}, n=10). Data pooled from three independent experiments. FIG. 1H shows the blood glucose levels measured over time following i.p. insulin (0.75 U/kg) injection during insulin-resistance test (WT, n=9; T-Myd88^{-/-, n=}10). Data pooled from three independent experiments. FIG. 1I shows a representative hematoxylin and eosin staining of liver and VAT tissue from WT and T-Myd88^{-/-} mice, taken with 20× magnification. Scale bar indicates 100 μm. FIG. 1J shows the percentage of weight gained of WT and T-Myd88^{-/-} mice fed a control or HFD (WT CTRL, n=8; WT HFD, n=15; T-Myd88^{-/-} CTRL, n=9; T-Myd88^{-/-} HFD, n=13). P-value<0.05 (*); P-value<0.01 (**); P-value<0.001 (***); P-value<0.0001 (****) using a two-tailed, unpaired t test (B-G) and a repeated measures ANOVA (H, J). Error bars indicate SD.

FIGS. 2A-D show that microbiota is required for weight gain associated with T-Myd88^{-/-} mice. FIG. 2A shows the grams of weight gained measured over time (mean +/−SD). FIG. 2B shows the total weight gained (AUC). FIG. 2C shows the grams of VAT. FIG. 2D shows the final body fat percentage when WT and T-Myd88^{-/-} mice were fed HFD with or without antibiotics (WT CTRL, n=5; TMYD CTRL, n=4; WT ABX, n=5, TMYD ABX, n=5). Representative of two independent experiments. P-value<0.05 (*); P-value<0.01 (**); P-value<0.001 (***); P-value<0.0001 (****) using a repeated measures ANOVA (A) and a two-tailed, unpaired t test (B-D).

FIGS. 3A-F shows the loss of diversity and Clostridia abundance are associated with weight gain in T-Myd88^{-/-} mice. FIG. 3A shows the PCoA plot and FIG. 3B shows the number of observed OTUs from the ileal microbiota of indicated animals (WT, n=8; T-Myd88^{-/-}, n=7). FIG. 3C shows the top ten bacterial genera influencing mean accuracy of random forest classification between WT and T-Myd88^{-/-} ileal microbiota. Genera with enriched relative abundance in WT animals are shaded blue, genera with enriched relative abundance in T-Myd88^{-/-} animals are shaded red (WT, n=8; T-Myd88^{-/-}, n=7). FIG. 3D shows the top ten bacterial genera influencing standard error in random forest linearization of weight gain and ileal microbiota. Genera with enriched relative abundance in WT animals are shaded blue, genera with enriched relative abundance in T-Myd88^{-/-} animals are shaded red (WT, n=8; T-Myd88−/−, n=7). FIG. 3E shows a volcano plot of the ratio of bacterial UniRef90 gene family transcript abundances in ileal samples (n=6 per cohort). FIG. 3F shows the mapped reads per million of significantly different species from WT and T-Myd88^{-/-} ileal microbiota transcripts (n=6 per genotype). Error bars indicate SD. Data in FIGS. 3A, 3B, 3C and 3D are from one experiment and data from FIGS. 3E, and 3F are from one experiment. P-value<0.05 (*); P-value<0.01 (**); P-value<0.001 (***); P-value<0.0001 (****) using permanova (A) and two-tailed unpaired t test (B, F).

FIGS. 4A-G show that manipulation of gut microbiota influences T-Myd88^{-/-} associated weight gain. FIG. 4A shows the area under the curve (AUC) of weight gained and FIG. 4B shows the relative abundance of Desulfovibrio in WT and T-Myd88^{-/-} mice maintained in separate cages or cohoused and fed a HFD (n=4 per genotype). Representative of two independent experiments. FIG. 4C shows the relative abundance of indicated bacteria within fecal samples from SPF mice colonized with or without D. desulfuricans (n=5 per genotype). FIG. 4D shows the relative abundance of indicated bacteria from 16S sequencing in germfree mice colonized with the Clostridia consortium alone or together with D. desulfuricans (n=5 per cohort). Error bars indicate SD. FIGS. 4E-G) show T-Myd88^{-/-} mice that were gavaged with vehicle control or spore-forming Clostridia consortium (Vehicle (CTRL), n=4; Clostridia consortium, n=5). Representative of two independent experiments. (E) AUC of weight gained. FIG. 4F shows the total fat percentage as measured by NMR. (G) Grams of VAT. P-value<0.05 (*); P-value<0.01 (**); P-value<0.001 (***); P-value<0.0001 (****) using a two-tailed, unpaired t test (A,E-G) and a Mann-Whitney U test (B-D).

FIGS. 5A-H show that TFH cell regulation of the microbiota prevents obesity. FIGS. 5A-H show Tcrb^{-′-} mice that were given a mixture of WT and T-Myd88^{-/-} microbiota one week before being given either WT or T-Myd88^{-/-} T cells. Mice were then individually housed for 8 weeks and measured for weight gain and microbiota composition while being fed a normal chow (n=6 per cohort). FIG. 5A show the area under the curve (AUC) analysis of weight gained. FIG. 5B shows a representative flow cytometry plot was previously gated on SYBR Green+ cells in order to quantify the percentage of antibody bound bacteria at 8 weeks. Rag^{-′-} feces control (grey shaded area); Tcrb^{-′-} feces (gray line); WT (blue line); T-Myd88^{-/-} (red line). Quantitation of multiple animals to the right. FIG. 5C shows a violin plot of Bray-Curtis distances between microbiota of TCRβ^{-′-} mice given WT or T-Myd88^{-/-} T cells at days 0, 7 and 28. FIG. 5D shows a correlation between Desulfovibrionaceae abundance and Clostrideaceae abundance in Tcrb ^{-′-} mice given WT or T-Myd88^{-/-} CD4⁺ T cells (n=12). FIG. 5E shows the relative abundance of Clostridiaceae (4 weeks). Error bars indicate SD. FIG. 5F shows IgA-bound and IgA-unbound bacteria were analyzed from cecal contents of Tcrb^{-/-} mice given WT or T-Myd88−′−

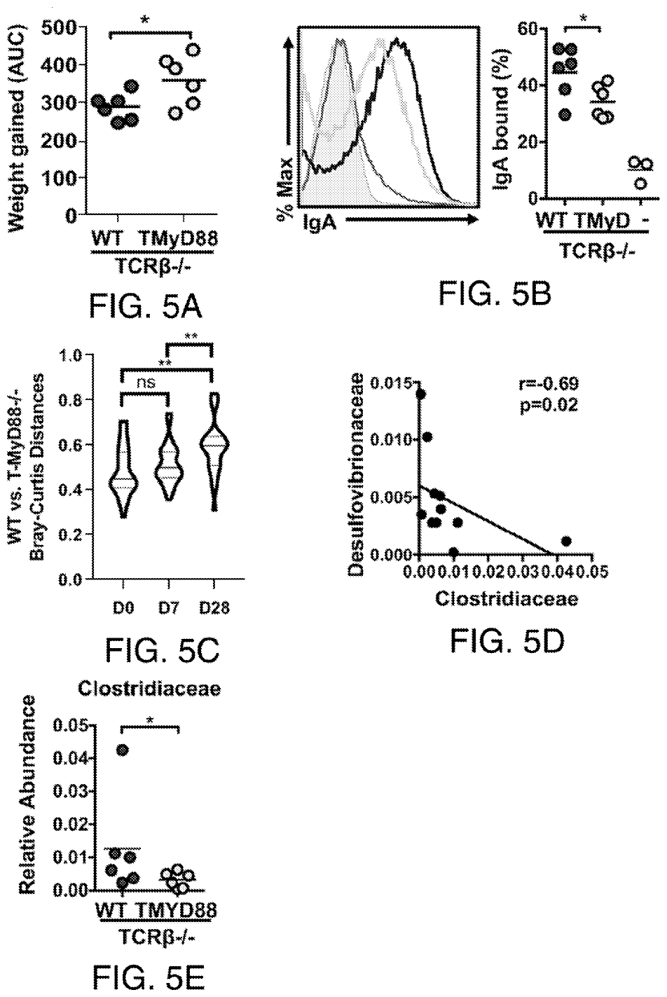

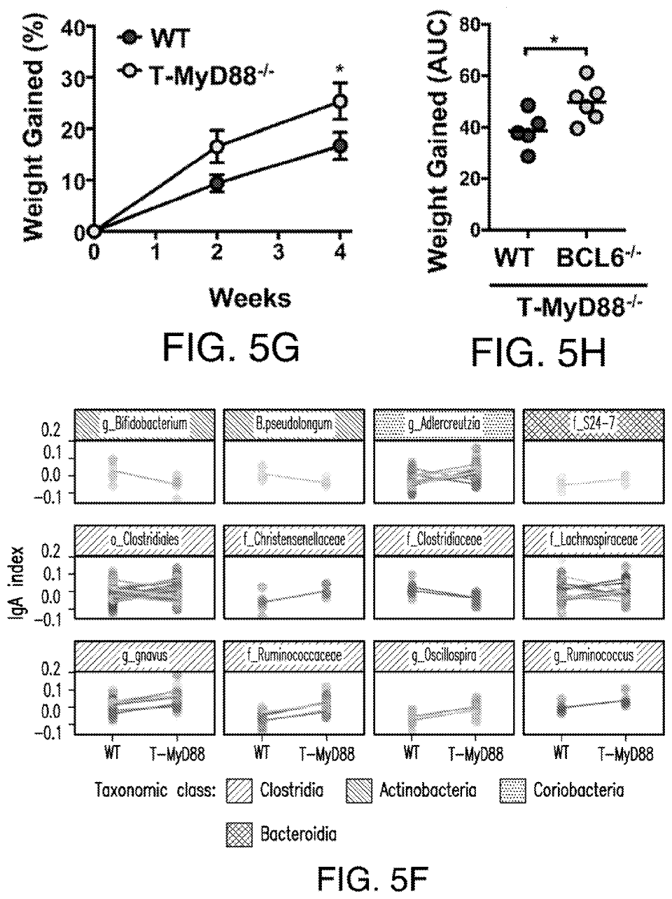

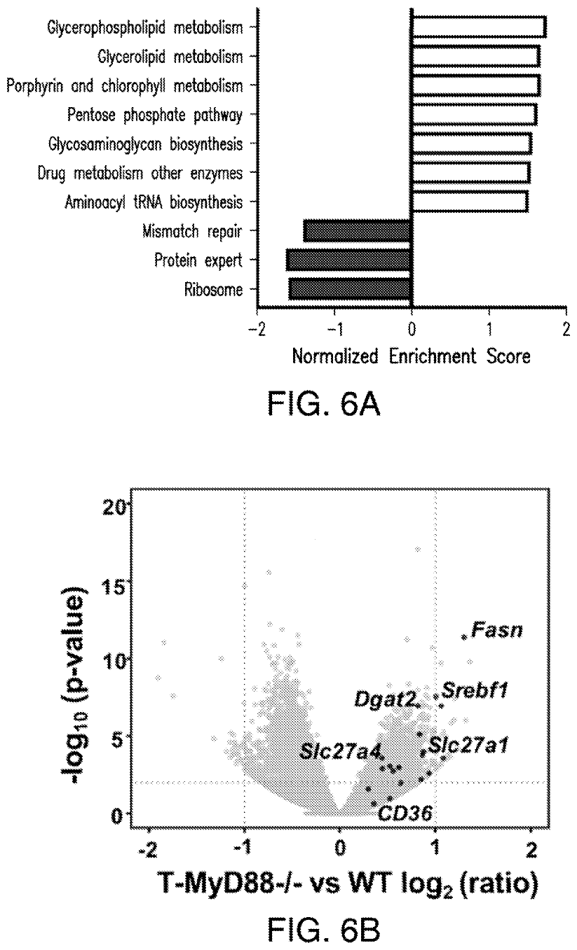

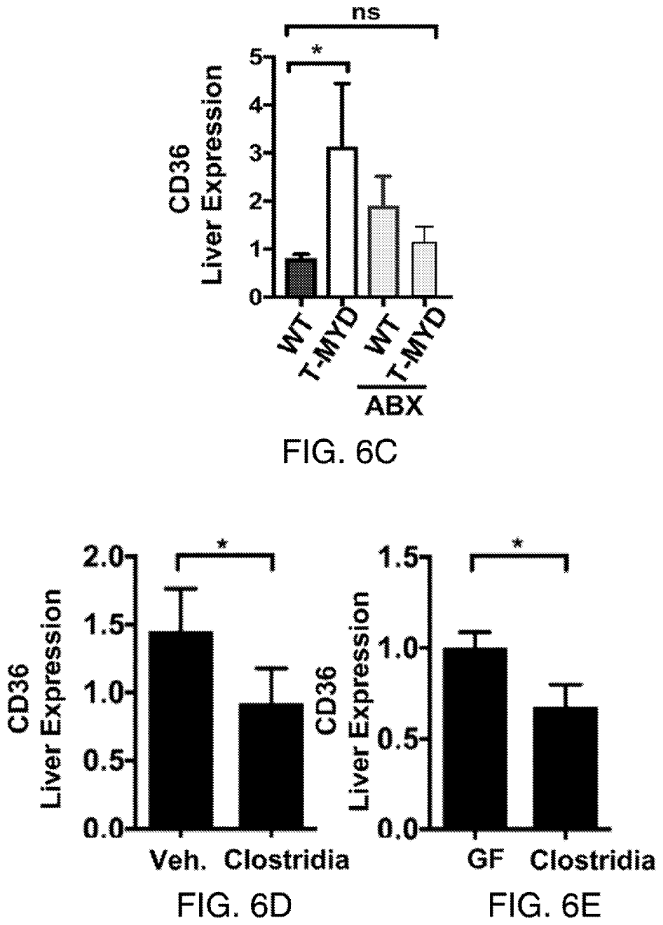

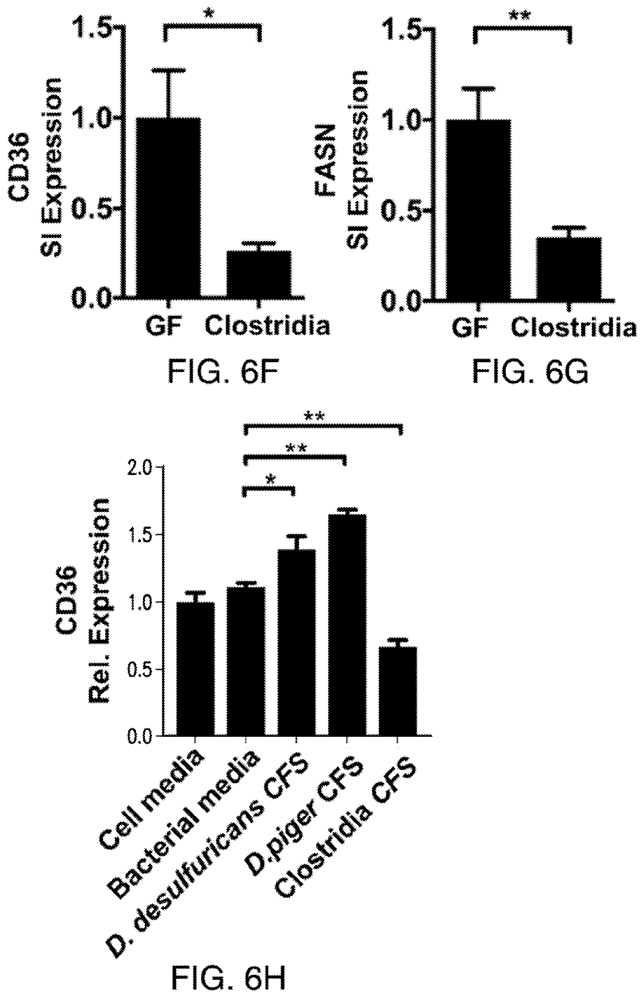

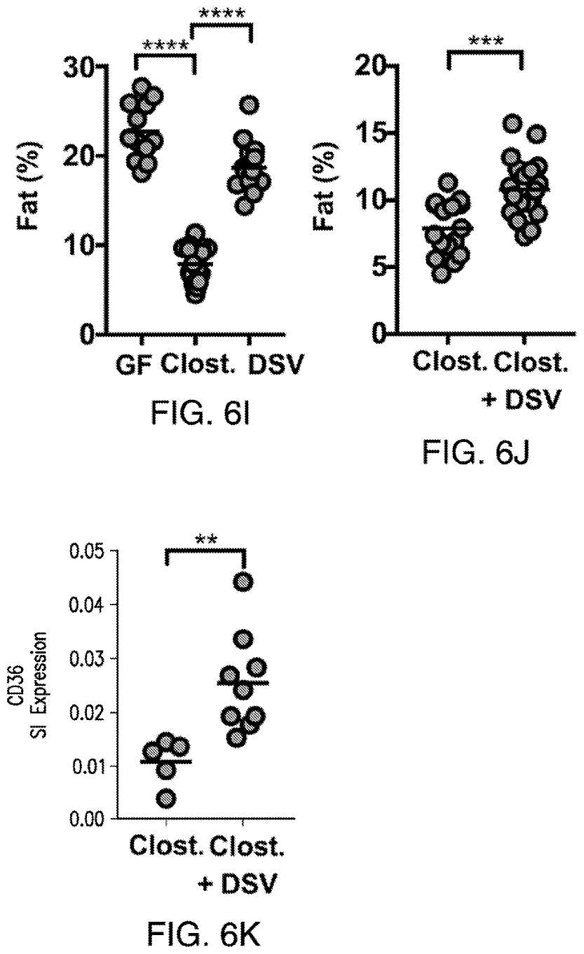

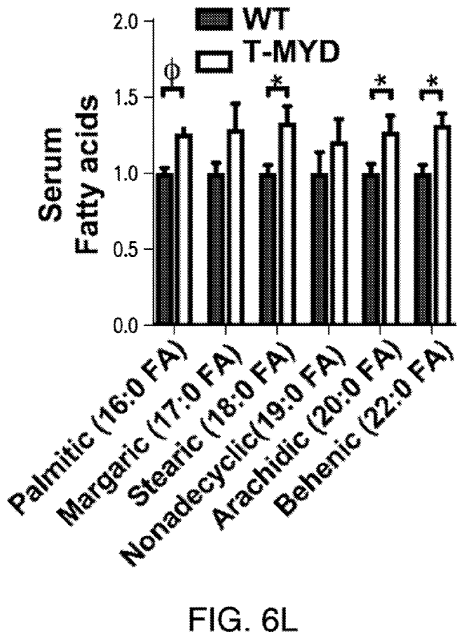

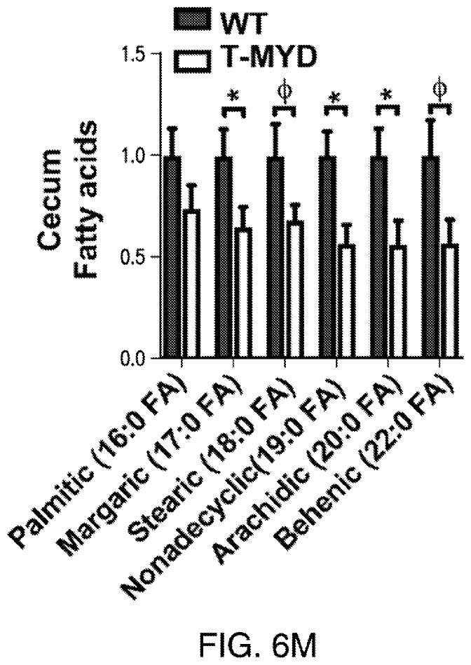

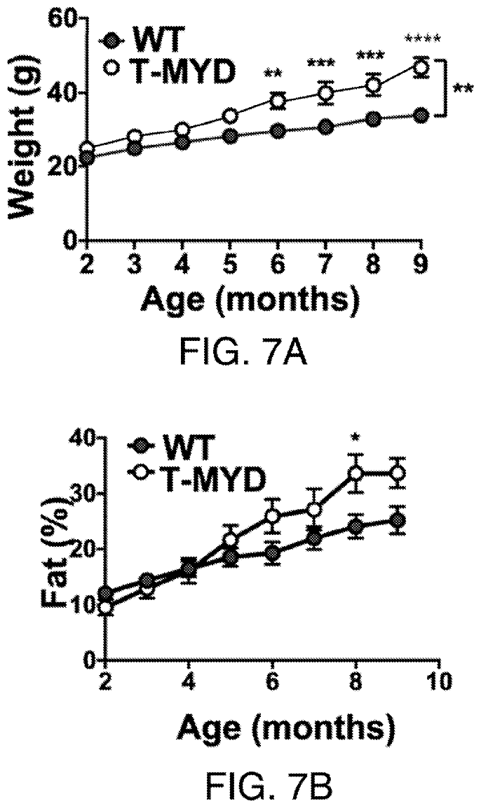

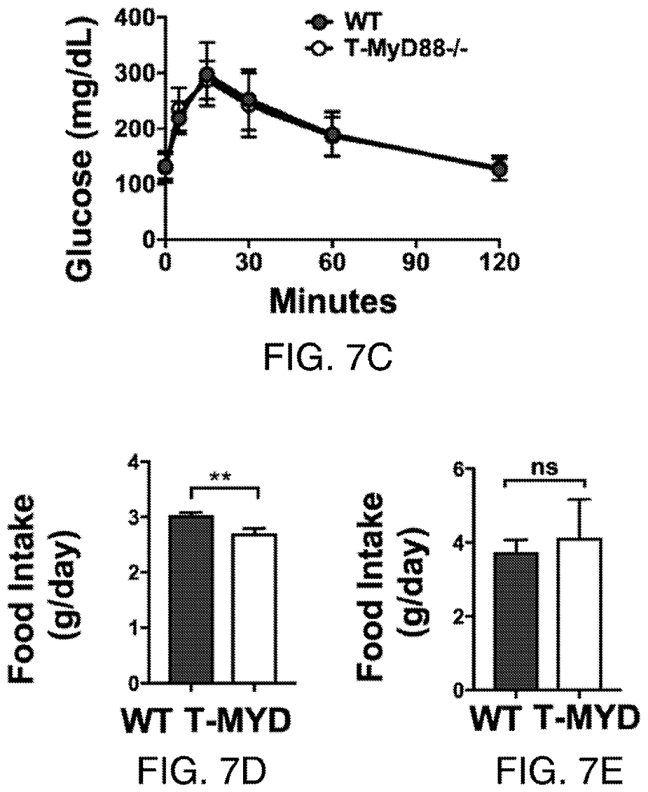

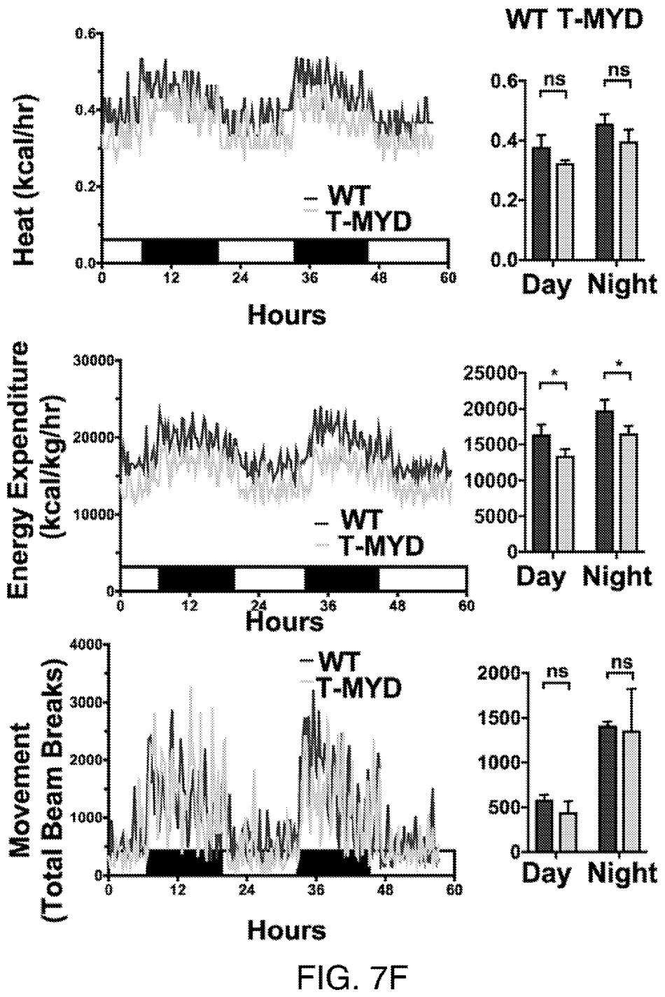

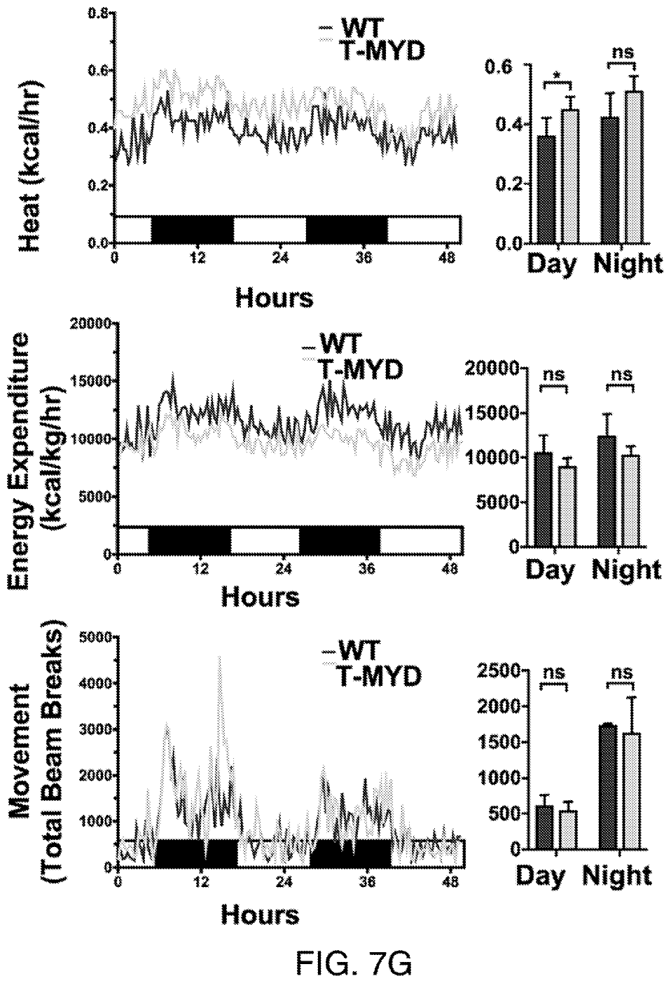

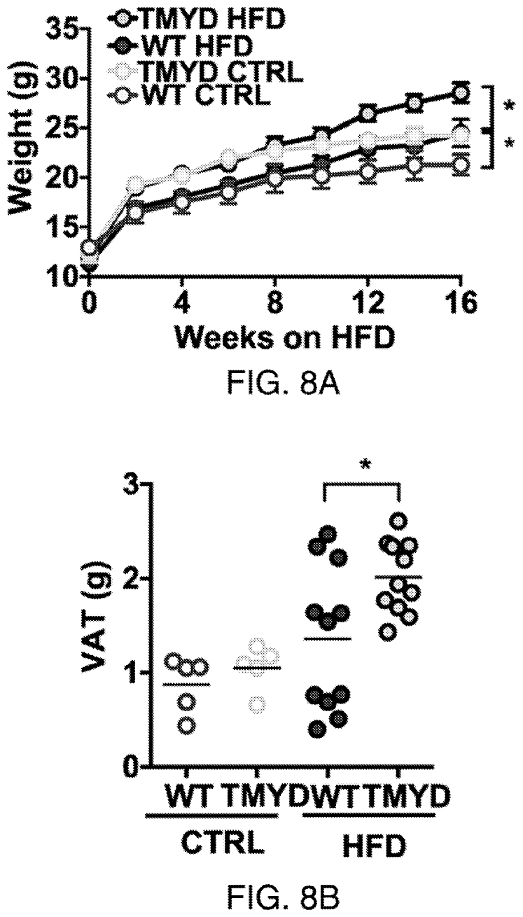

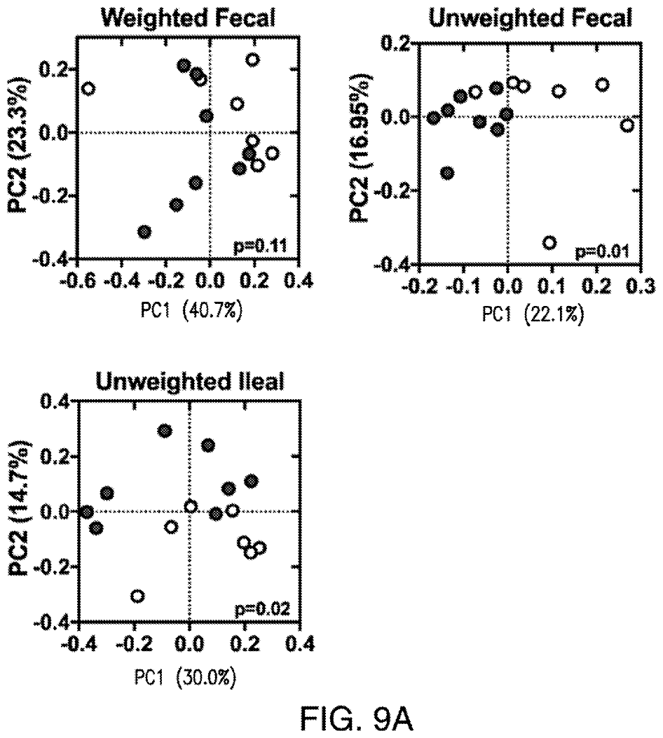

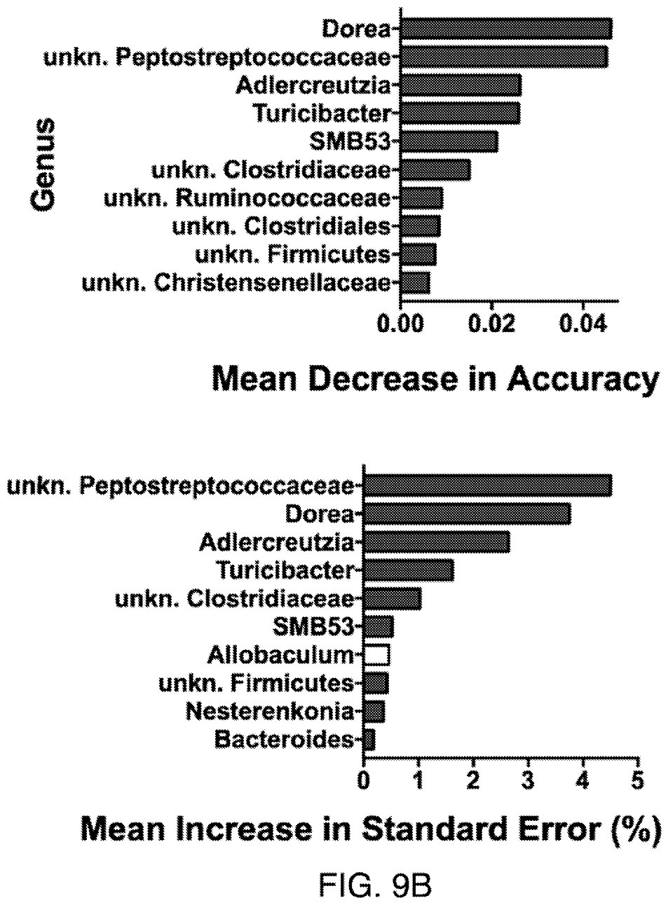

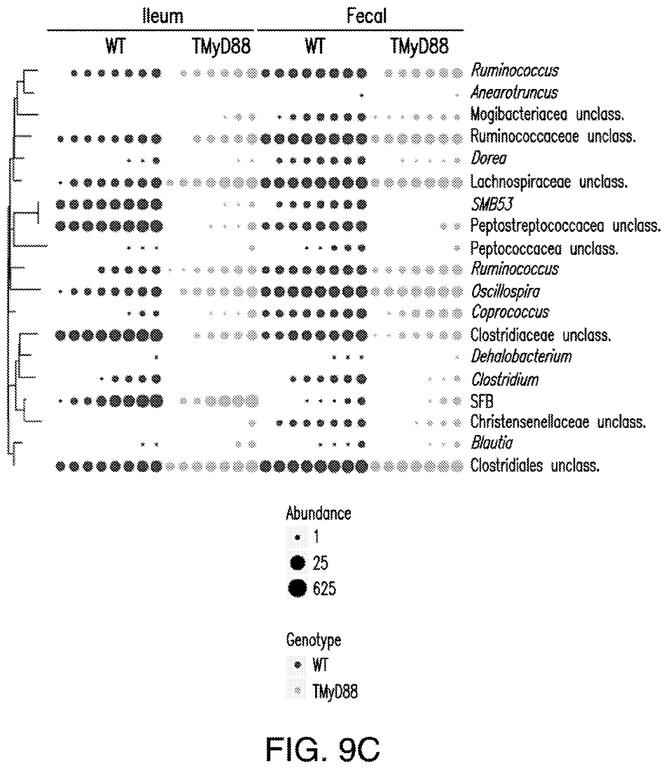

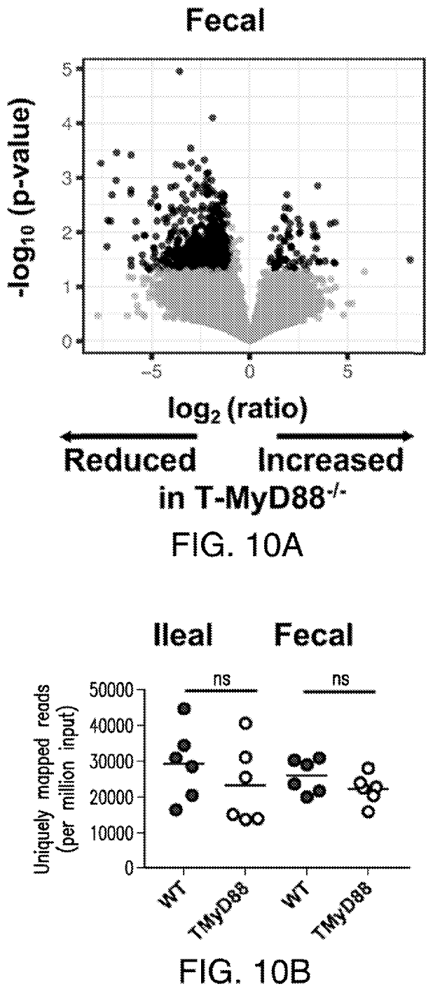

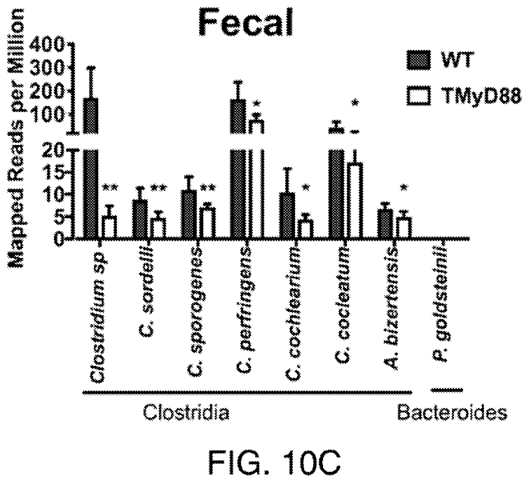

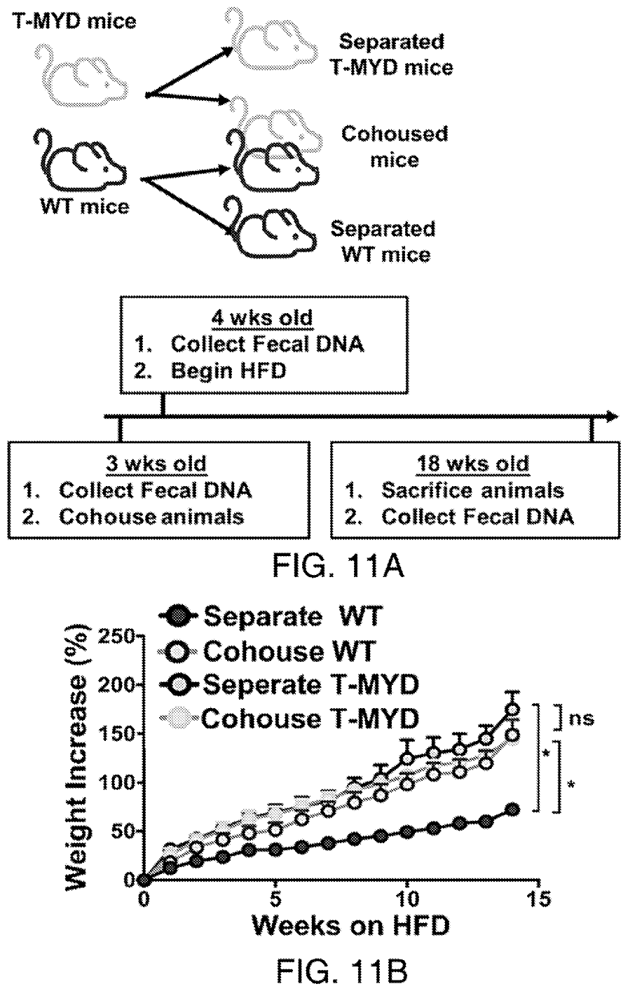

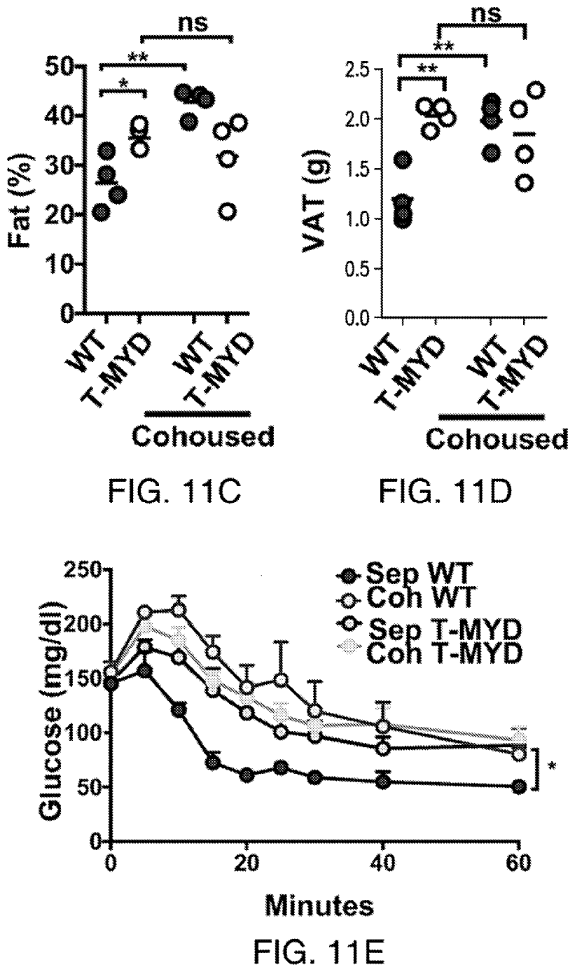

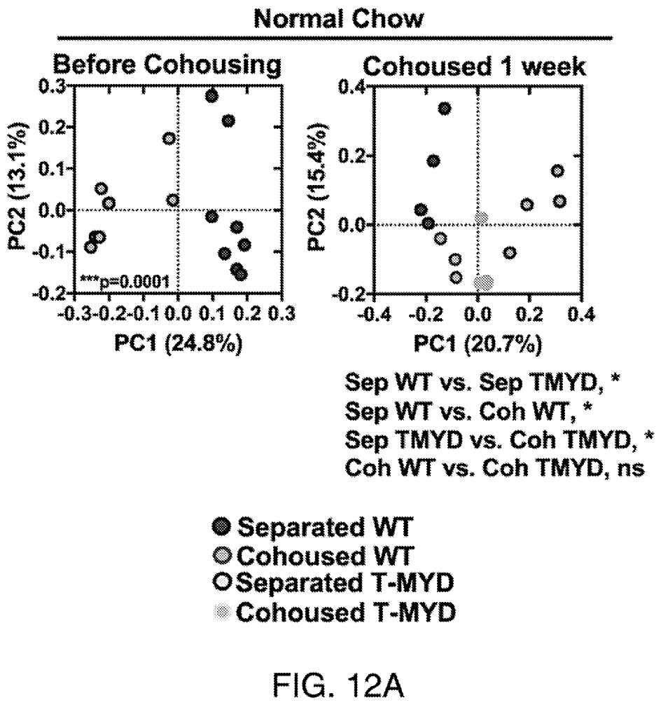

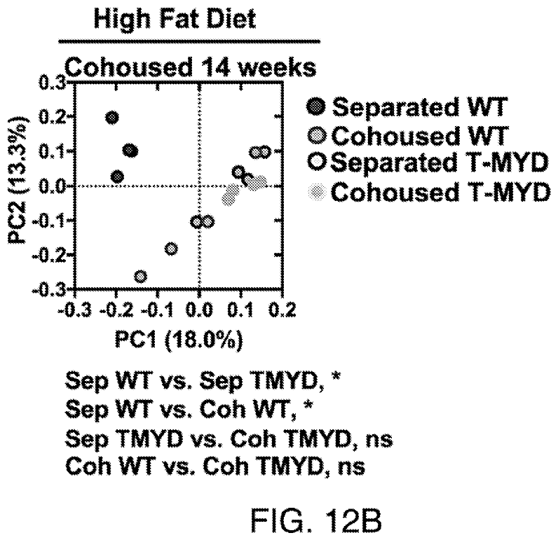

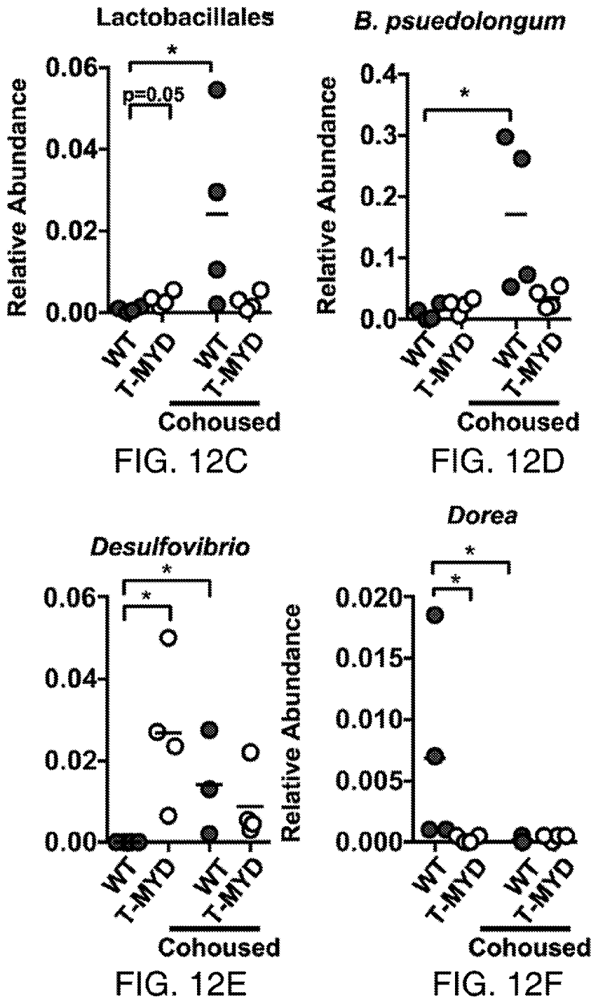

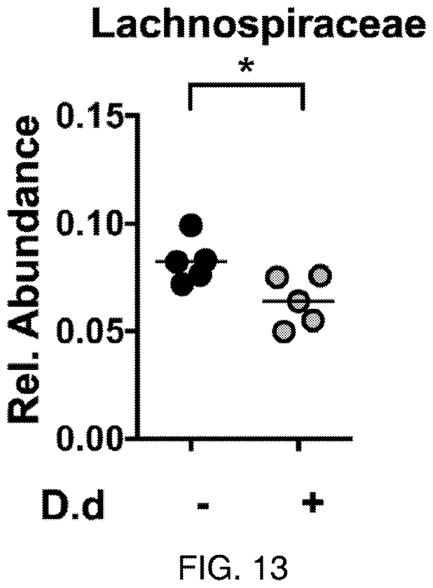

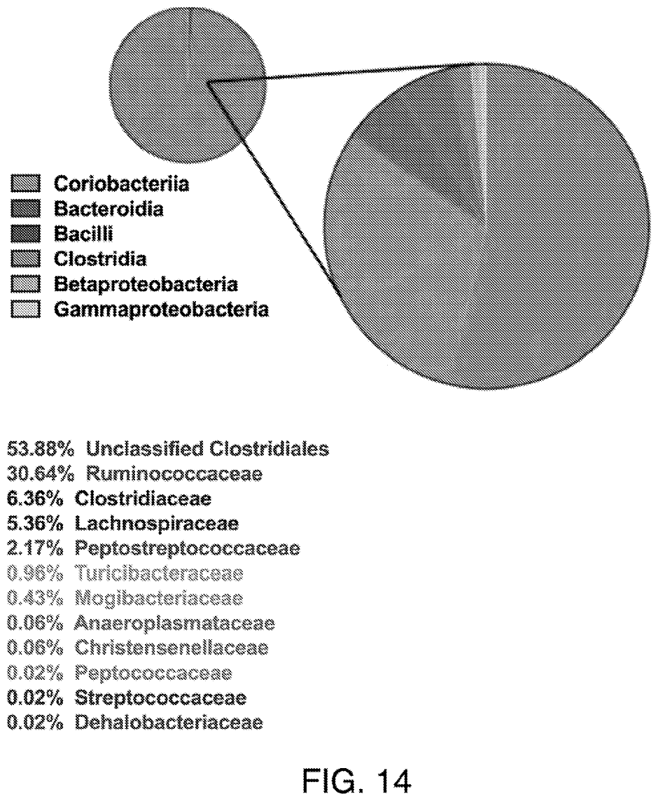

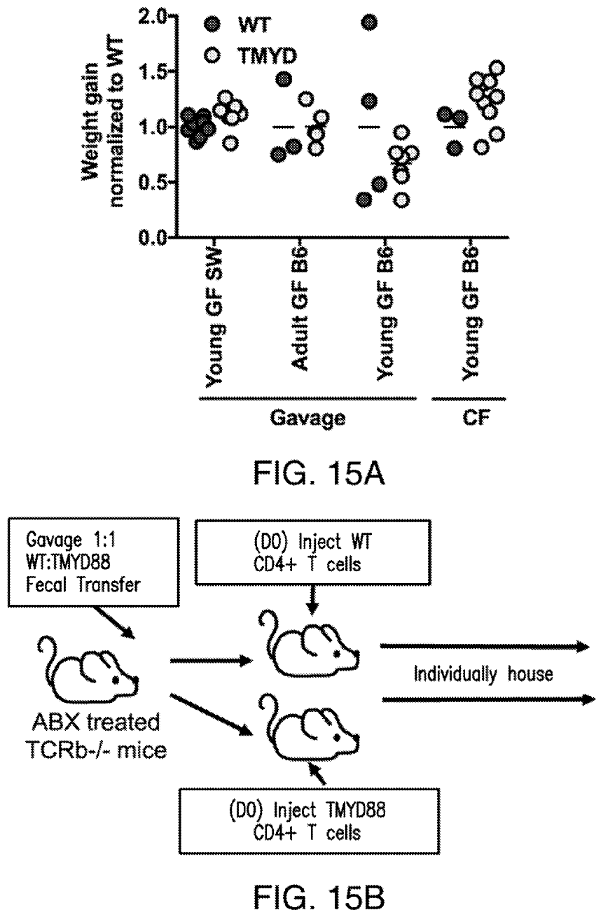

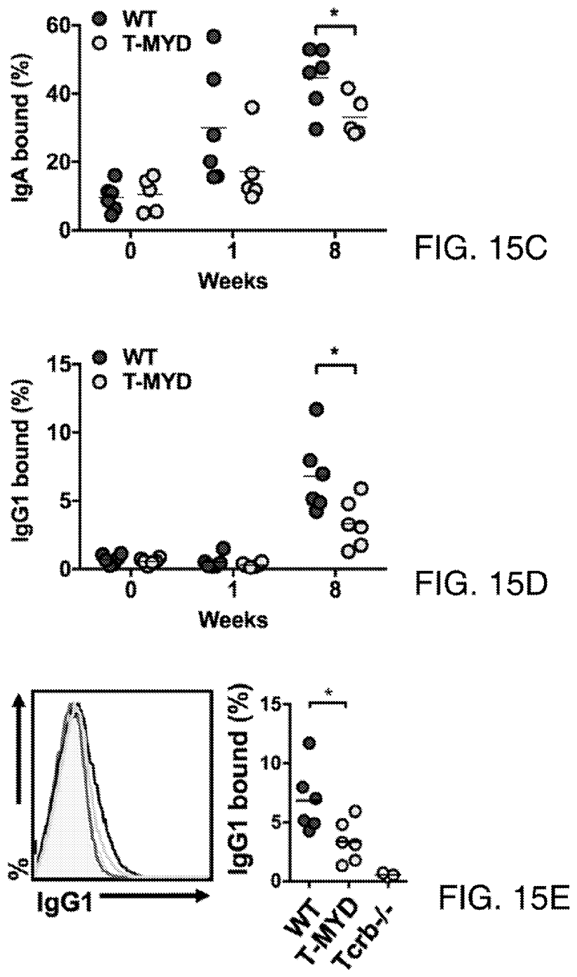

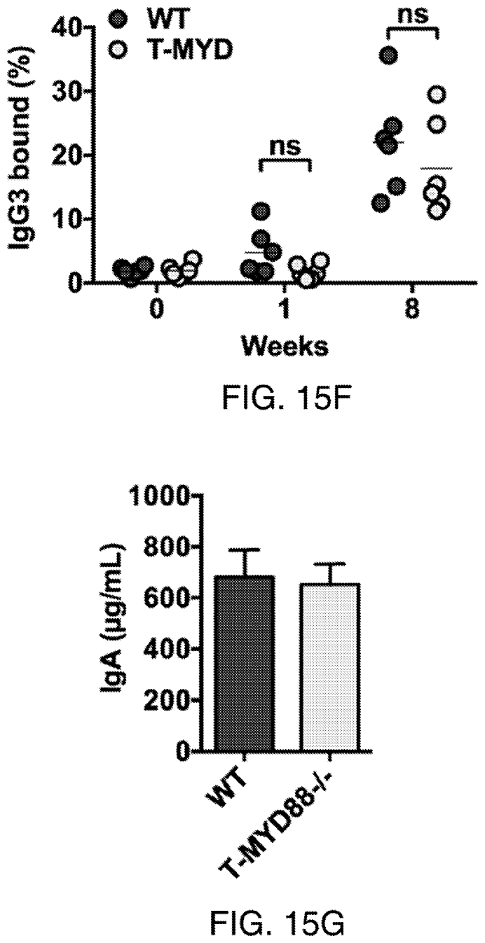

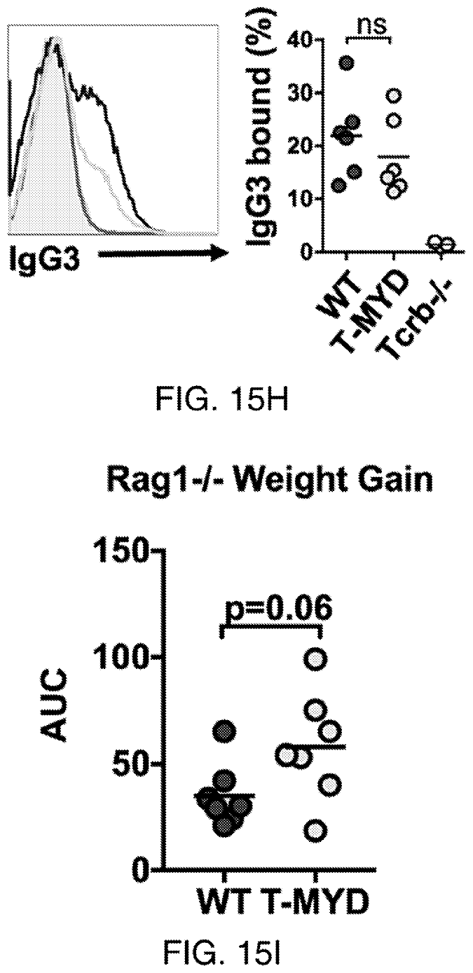

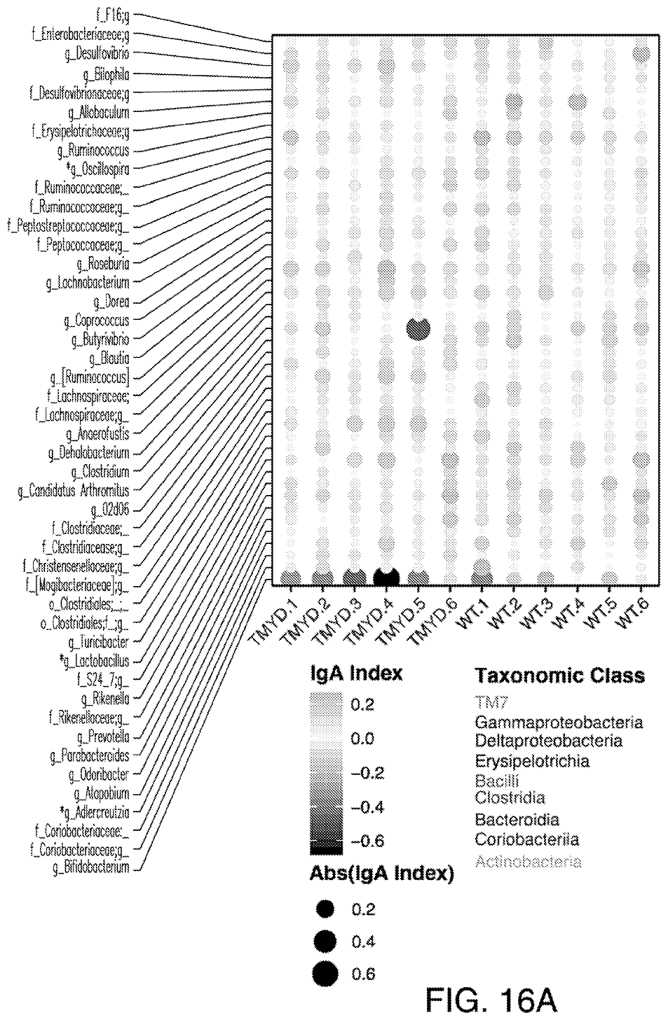

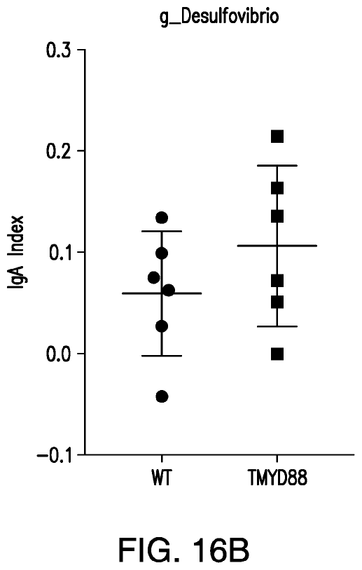

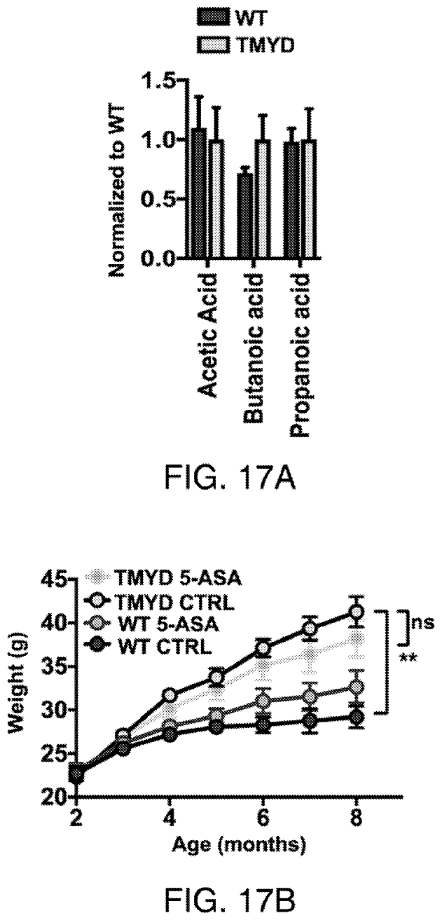

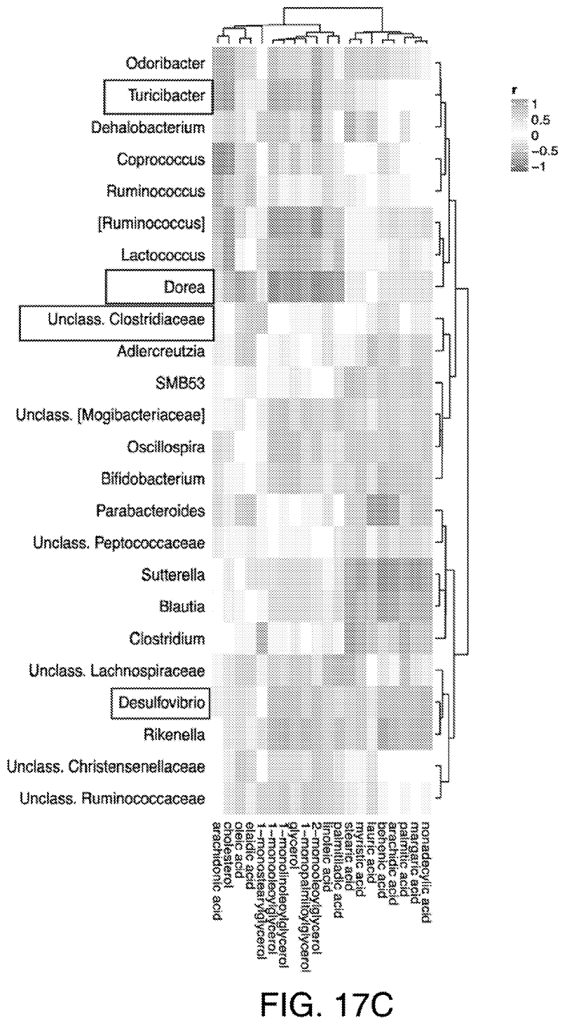

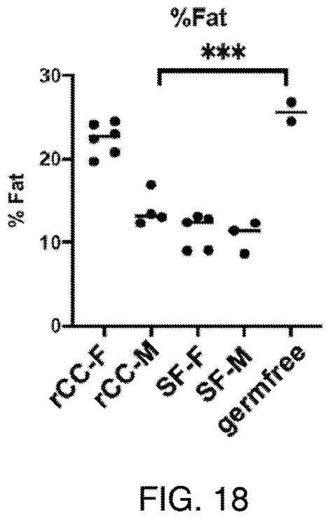

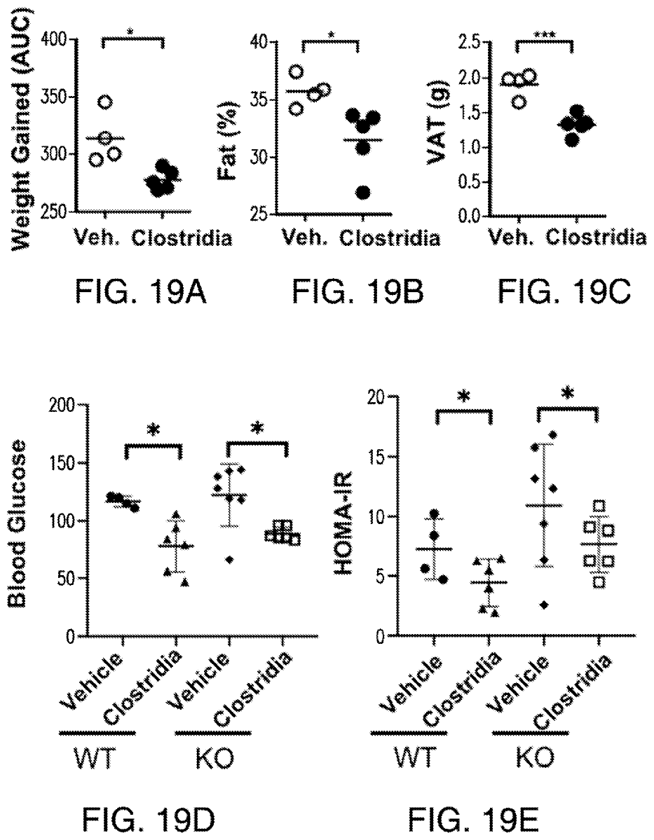

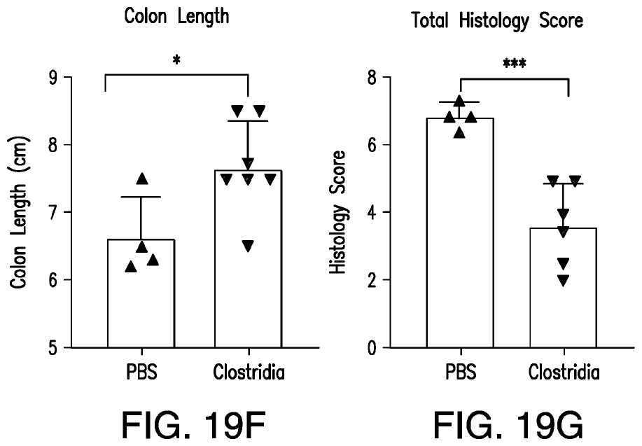

CD4⁺ T cells. An IgA index was calculated for each OTU to show differences in binding. Positive values indicate enrichment in the bound fraction and negative values enrichment in the unbound fraction. All OTUs with statistically significant differences are shown (p<0.05, Wilcoxon rank sum test). Each panel groups OTUs with the same taxonomic call according to their finest classification level (genus (g), family (f), or order (o)). Each dot represents an individual animal, while different colors within a panel distinguish OTUs within a taxa, and each line connects the means from each OTU. Data from one experiment. FIG. 5G shows the percent weight gained in Rag1^{-′-} mice colonized with WT or T-Myd88^{-/-} fecal microbiota (n=7 per cohort). Representative of two independent experiments. FIG. 5H shows the AUC of weight gained in T-Myd88^{-/-} mice receiving donor WT or Bcl6^{-/-} T cells and fed a normal chow (WT donor, n=5; T-Myd88^{-/-} donor, n=6). Representative of two independent experiments. P-value<0.05 (*); P-value<0.01 (**); P-value<0.001 (***); P-value<0.0001 (****) using a two-tailed, unpaired t test (A, B, H),repeated measures ANOVA with Tukey's multiple comparison (C), Spearman's rank-order correlation (D), a repeated measures ANOVA Sidak's correction for multiple comparisons (G) and a Mann-Whitney U test (E). Error bars indicate SD (D,G).

FIGS. 6A-M show that Clostridia inhibit lipid absorption within the intestine. FIG. 6A shows GSEA analysis from RNA expression in livers from 1-year-old WT and T-Myd88^{-′-} mice, pathways that had a significant FDR of 0.25 or smaller were included. FIG. 6B shows a Volcano plot of ratio of liver transcripts. Highlighted genes are involved in lipid metabolism. FIG. 6C shows Cd36 RNA expression within livers of WT and T-Myd88^{-′-} mice fed HFD with or without antibiotics (ABX) (WT, n=5; T-Myd88^{-/-}, n=4; WT ABX, n=5, T-Myd88^{-/-} ABX, n=5). Representative of two independent experiments. FIG. 6D shows Cd36 RNA expression in livers of T-Myd88^{-′-} mice gavaged with vehicle control or spore-forming Clostridia consortium (control n=4; Clostridia consortium, n=5). Representative of two independent experiments. FIGS. 6E-G show germfree mice with or without colonization of a Clostridia consortium (GF, n=8; Clostridia, n=10). FIG. 6E shows Cd36 RNA expression in the liver. FIG. 6F shows Cd36 RNA expression in the small intestines (SI). FIG. 6G shows Fasn RNA expression in the SI. FIG. 6H shows Cd36 RNA expression in MODE-K cells incubated for 4 hours with media or bacterial cell-free-supernatant (CFS). Representative of three independent experiments. FIG. 6I that germfree mice were associated with the Clostridia consortia or two Desulfovibrio species (D. piger and D. desulfuricans). Body fat percentage was measured by NMR analysis. (Germfree mice n=12; Clostridia, n=16, Desulfovibrio, n=14). FIGS. 6J,K show germfree mice were associated with the Clostridia consortia with or without D. piger and D. desulfuricans (DSV). Body fat percentage was measured by NMR (Clostridia alone n=16; Clostridia+DSV n=21) (J) and Cd36 within the small intestine by q-PCR (FIG. 6K). FIGS. 6L,M show GC-MS-detected metabolites within serum and cecum contents of WT and T-Myd88^{-/-} mice fed HFD (n=6 per cohort). P-value of <0.06 ((p), P-value<0.05 (*); P-value<0.01 (**); P-value<0.001 (***); P-value<0.0001 (****) a two-tailed, unpaired t test (C-G, I-K), and one-way ANOVA Sidak's correction for multiple comparisons (FIG. 6H). Data are presented as mean+/−SD.

FIGS. 7A-G show that mice lacking Myd88 signaling within T cells develop age-associated obesity. FIG. 7A shows that weight gained as mice age, starting at 2 months of age (WT, n=8; T-Myd88^{-/-} n=7). FIG. 7B shows the percentage of fat gained as mice age, starting at 2 months of age (WT, n=8; T-Myd88^{-/-}, n=7). FIG. 7C shows blood levels of glucose (mg/dL) measured over time following i.p. glucose (1 mg/g) injection during glucose tolerance test of 1-year-old WT and T-Myd88^{-/-} mice. FIG. 7D shows grams of food intake per mouse while being fed normal chow at 2 months (n=3 per cohort). FIG. 7E shows grams of food intake per mouse while being fed normal chow 1-year-old mice (n=5 per group. FIG. 7F shows heat, energy expenditure, and total movement of 2-month-old (n=3 per group). FIG. 7G shows heat, energy expenditure, and total movement of 1-year-old mice (n=5 per group). Statistics: p-value<0.05 (*); p-value<0.01 (**); p-value<0.001 (***) using a repeated measures ANOVA with Sidak's correction for multiple comparisons (FIGS. 7A, 7B, 7C), two-tailed, unpaired t test (FIGS. 7D-G). Error bars indicate SD.

FIGS. 8A-B show that obesity in T-Myd88^{-/-} mice is accelerated by increased dietary intake of fat. FIG. 8A shows the weight of animals over-time after high fat diet feeding. FIG. 8B shows viceral fat mass in age matched indicated animals on control chow or 16 weeks post HFD feeding. Statistics: P-value<0.05 (*); P-value<0.01 (**); P-value<0.001 (***) using a repeated measures ANOVA (FIG. 8A) and two-tailed, unpaired t test (FIG. 8B). Error bars indicate SD.

FIGS. 9A-C show that changes to microbial composition within T-Myd88^{-/-} mice is associated with spontaneous weight gain. FIG. 9A show beta-diversity analysis of ileal and fecal 16S sequencing samples from 1-year-old WT and T-Myd88^{-′-} mice, measured by unweighted unifrac and weighted unifrac (WT, n=8; T-Myd88^{-′-}, n=7). FIG. 9B show random forest analysis of microbial communities. FIG. 9C show the number and relative abundance of Clostridia OTUs in fecal and ileal microbiota (WT, n=8; T-Myd88 n=7). Statistics: p-value<0.05 (*); p-value<0.01 (**); p-value<0.001 (***) using a PERMANOVA (C).

FIGS. 10A-C show the transcriptomic data of microbiota from WT or T-Myd88^{-/-} mice. FIG. 10A show a volcano plot of ratio of bacterial UniRef90 gene family transcript abundances in fecal samples. FIG. 10B show the uniquely mapped reads per million in WT and T-Myd88^{-′-} mice ileum and feces. FIG. 10C show mapped reads per million of significantly different species from WT and T-Myd88^{-′-} fecal transcripts (n=6 per genotype). Statistics: p-value<0.05 (*); p-value<0.01 (**); p-value<0.001 (***) using a two-tailed, unpaired t test. Error bars indicate SD.

FIGS. 11A-E show that dysbiosis within T-Myd88^{-/-} mice transfers obesity to WT animals during co-housing. FIG. 11A show a schematic of cohousing experiment and schematic of timeline for cohousing experiment. FIG. 11B shows percent weight increase, FIG. 11C shows percent fat, and FIG. 11D shows the grams of VAT in separated or cohoused WT and T-Myd88^{-/-} mice fed a HFD (n=4 per cohort). Representative of two independent experiments. FIG. 11E show blood levels of glucose (mg/dL) measured over time following i.p. glucose (1 mg/g) injection during glucose tolerance test of separated or cohoused WT and T-Myd88^{-/-} mice fed a HFD (n=4 per cohort). Statistics: p-value<0.05 (*); p-value<0.01 (**); p-value<0.001 (***) repeated measures ANOVA (B,E), two-tailed, unpaired t test (C,D). Error bars indicate SD.

FIGS. 12A-F show the determination of transmissible organisms during co-housing. FIGS. A and B show beta diversity measured by unweighted Unifrac analysis of separated or cohoused WT and T-Myd88^{-′-} mice fed a normal chow both prior to cohousing and one week following cohousing (FIG. 12A) and then after 14 weeks of HFD (FIG. 12B). FIGS. 12C and D show the relative abundance of indicated organisms within fecal 16S sequencing samples from separated or cohoused WT and T-Myd88^{-′-} at the final time point (n=4 per cohort). FIG. 12E shows the relative abundance of Desulfovibrio within fecal samples from indicated animals just one week after co-housing. FIG. 12F shows the relative abundance of Dorea in indicated animals after 12 weeks of cohousing. Statistics: p-value<0.05 (*); p-value<0.01 (**); p-value<0.001 (***) permanova (A,B) and a Mann-Whitney U test (C-F).

FIG. 13 shows that expansion of Desulfovibrio leads to loss of Clostridia. The graphs shows the relative abundance of Lachnospiraceae within fecal 16S sequencing samples from mice colonized with or without D. desulfuricans and fed a HFD (n=5 per cohort). Statistics: p-value<0.05 (*); p-value<0.01 (**); p-value<0.001 (***) Mann-Whitney U test.

FIG. 14 shows the microbiota composition from germfree mice colonized with spore-forming microbes. Parts of whole graph from 16s sequencing of fecal microbiota of germfree mice colonized with spore-forming clostridia consortium.

FIGS. 15A-I show that T cell shaping of the microbiota is associated with spontaneous weight gain. FIG. 15A shows weight gained in germfree mice given WT or T-Myd88^{-/-} microbiota through multiple methods of transfer (CF=cross fostered). FIG. 15B shows a schematic of experimental strategy. Tcrb^{-/-} animals were depleted of the microbiota by antibiotic treatment and subsequently gavaged with a 1:1 mixture of microbiota from WT or T-Myd88^{-/-} animals. WT or T-Myd88^{-/-} T cells were transplanted into T cell deficient animals. FIG. 15C shows Flow cytometry used to quantify the percentage of IgA bound bacteria within Tcrb^{-/-} mice given WT or T-Myd88^{-/-} cells at Day 0, Week 1, and Week 8 (n=6 per cohort). FIGS. 15D,E show flow cytometry used to quantify the percentage of IgG1 bound bacteria within Tcrb^{-/-} mice given WT or T-Myd88^{-/-} cells at Day 0, Week 1, and Week 8. FIG. 15F shows flow cytometry used to quantify the percentage of IgG3 bound bacteria within Tcrb^{-/-} mice given WT or T-Myd88^{-/-} cells at Day 0, Week 1, and Week 8. FIG. 15G shows the concentration of luminal IgA (μg/mL) was measured within Tcrb^{-/-} mice given WT or T-Myd88^{-/-} cells after 8 weeks using an ELISA. Error bars indicate SD. FIG. 15H shows a representative flow cytometry plot was previously gaited on SyBR Green⁺ cells in order to quantify the percentage of IgG3 bound bacteria within Tcrb^{-/-} mice given WT or T-Myd88^{-/-} cells after 8 weeks. FIG. 15I shows the AUC of weight gained of Rag1 −/− mice colonized with WT or T-Myd88^{-/-} fecal microbiota (n=7 per cohort). Statistics: p-value<0.05 (*); p-value<0.01 (**); p-value<0.001 (***) two-tailed, unpaired t test (A, C-I).

FIGS. 16A-B show IgA targeting of bacterial communities (FIG. 16A) IgA bound and unbound bacteria were analyzed from cecal contents of Tcrb^{-/-} given either WT or T-Myd88^{-/-} T cells. The IgA index was calculated for each genus in each animal (columns). Bubbles are colored by enrichment in the bound or unbound fraction and sized by the magnitude of enrichment (the absolute value of the IgA index). Genus taxa strings are colored according to their taxonomic class. Significantly differentially bound genera between genotypes are indicated (*, p<0.05; Wilcoxon rank sum test). FIG. 16B shows Desulfovibrio IgA targeting in this dataset. Error bars indicate SD.

FIG. 17A-C show that gut metabolites are associated with weight gain (FIG. 17A) GC-MS detected SCFAs of cecal contents of WT and T-Myd88^{-/-} mice (WT, n=3; T-Myd88^{-/-} n=5). FIG. 17B shows the grams of weight gained by WT and T-Myd88^{-/-} mice fed control diet or 5-ASA diet, starting at 2 months of age (WT CTRL, n=3; WT 5-ASA, n=4; T-MYD CTRL, n=3; TMYD 5-ASA, n=4). Error bars indicate SD. FIG. 17C shows the spearman rank order correlation between relative abundances of Clostridia and fatty acid and monoacylglycerol metabolites (n=12). Statistics: p-value<0.05 (*); p-value<0.01 (**); p-value<0.001 (***) two-tailed, unpaired t test (A), and repeated measures ANOVA (B).

FIG. 18 shows a refined Clostridia consortia (rCC-4) of 4 strains (i.e. Clostridia anaerovorax strain, Clostridium XIVa, Clostridium IV, and Lachnospiraceae spps) is sufficient to reduce adiposity. Female (F) and male (M) germfree mice were colonized with the 4 strains cultured (rCC) from the more complex Clostridia consortia and analyzed by NMR 4 weeks after colonization. rCC-4 reduced adiposity to the same degree as the complex Clostridia consortia in males but not females.

FIGS. 19A-G show that Clostridia treatment improves MetS and IBD. FIGS. 19A-C show T-MyD88^{-/-} mice gavaged with vehicle control or spore-forming Clostridia consortium every third day for three months while on a HFD. FIG. 19A, weight gained; and FIG. 19B, total fat percentage as measured by NMR. C, visceral adipose tissue. FIG. 20D, and FIG. 20E both show wild-type (WT) and T-MyD88^{-/-} (KO) placed on a HFD and treated with Clostridia as in FIG. 19A and FIG. 19D, Fasting Glucose. FIG. 19E shows HOMA-IR values.

FIG. 19F and FIG. 19G show animals provided orally gavaged with the Clostridia consortia every other day while on three cycles of DSS colitis (5 days of DSS and 10 days of water). F, is colon length and G is pathology scores from H&E stained sections of colons. p-value<0.05 (*); p-value<0.001.

### DETAILED DESCRIPTION

The present disclosure can be understood more readily by reference to the following detailed description of the invention, the figures and the examples included herein.

Before the present methods and compositions are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile, cerebral spinal fluid) that contains cells or cell components. In some aspects, the sample can be taken from the brain, spinal cord, cerebral spinal fluid or blood.

As used herein, the term “subject” refers to the target of administration, e.g., a human. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment for multiple sclerosis, such as, for example, prior to the administering step. In some aspects of the disclosed methods, the “patient” has been diagnosed with a need for treatment for a type II diabetes, obesity, or inflammatory bowel disease, such as, for example, prior to the administering step.

As used herein, the term “normal” refers to an individual, a sample or a subject that does not have type II diabetes, obesity, or inflammatory bowel disease or does not have an increased susceptibility of developing type II diabetes, obesity, or inflammatory bowel disease.

As used herein, the term “susceptibility” refers to the likelihood of a subject being clinically diagnosed with a disease. For example, a human subject with an increased susceptibility for type II diabetes, obesity, or inflammatory bowel disease can refer to a human subject with an increased likelihood of a subject being clinically diagnosed with type II diabetes, obesity, or inflammatory bowel disease.

As used herein, the term “comprising” can include the aspects “consisting of” and “consisting essentially of”

As used herein, a “control” is a sample from either a normal subject or from tissue from a normal subject that does not have type II diabetes, obesity, or inflammatory bowel disease.

As used herein, “over-expression” means expression greater than the expression detected in a normal sample. For example, a nucleic acid that is over-expressed may be expressed about 1 standard deviation above normal, or about 2 standard deviations above normal, or about 3 standard deviations above the normal level of expression. Therefore, a nucleic acid that is expressed about 3 standard deviations above a control level of expression is a nucleic acid that is over-expressed.

As used herein, “treat” is meant to mean administer a compound or molecule of the invention to a subject, such as a human or other mammal (for example, an animal model), that has type II diabetes, obesity, or inflammatory bowel disease, in order to prevent or delay a worsening of the effects of the disease or condition, or to partially or fully reverse the effects of the disease.

As used herein, “prevent” is meant to mean minimize the chance that a subject who has an increased susceptibility for developing type II diabetes, obesity, or inflammatory bowel disease or will develop type II diabetes, obesity, or inflammatory bowel disease.

As used herein, the term “reference,” “reference expression,” “reference sample,” “reference value,” “control,” “control sample” and the like, when used in the context of a sample or expression level of one or more microbes refers to a reference standard wherein the reference is expressed at a constant level among different (i.e., not the same tissue, but multiple tissues) tissues, and is unaffected by the experimental conditions, and is indicative of the level in a sample of a predetermined disease status (e.g., not suffering from type II diabetes, obesity, or inflammatory bowel disease). The reference value can be a predetermined standard value or a range of predetermined standard values, representing no illness, or a predetermined type or severity of illness.

Compositions

The present disclosure is directed to compositions containing and methods of using bacterial isolates and communities. In particular, the present disclosure is directed to a composition containing one or more microorganisms from the bacterial consortia as disclosed herein, particularly in Table 1 or mixtures thereof. In a preferred embodiment, the composition will include two or more strains of bacterium having a 16S rDNA sequence comprising SEQ ID NOs: 1, 2, 3, and 4. The microorganisms can be characterized by an identifying 16S ribosomal gene sequence corresponding to, and at and least 98% identical to SEQ ID NOs 1-4.

Disclosed herein are newly identified bacterium. It was found that the bacterium having a 16S rDNA sequence comprising SEQ ID NOs: 1, 2, 3, and 4 belong to the genus Clostridium. In some aspects, the compositions described herein comprise at least one bacterium, wherein the bacteria is a Clostridium sp.

Clostridia anaerovorax. Disclosed herein is a bacteria, Clostridia anaerovorax. Clostridia anaerovorax as used herein refers to a bacteria having a 16S nucleic acid sequence sharing at least 98% sequence identity to SEQ ID NO: 1. Clostridia anaerovorax has NRRL or ATCC Accession number ______. Clostridia anaerovorax was isolated from a fecal pellet and luminal content from the lower the small intestine of CD4-Cre⁺ mice (WT).

Clostridium XIVa. Disclosed herein is a bacteria, Clostridium XIVa. Clostridium XIVa as used herein refers to a bacteria having a 16S nucleic acid sequence sharing at least 98% sequence identity to SEQ ID NO: 2. Clostridium XIVa has NRRL or ATCC Accession number ______. Clostridium XIVa was isolated from a fecal pellet and luminal content from the lower the small intestine of CD4-Cre⁺ mice (WT).

Clostridium IV. Disclosed herein is a bacteria, Clostridium IV. Clostridium IV as used herein refers to a bacteria having a 16S nucleic acid sequence sharing at least 98% sequence identity to SEQ ID NO: 3. Clostridium IV has NRRL or ATCC Accession number ______. Clostridium IV was isolated from a fecal pellet and luminal content from the lower the small intestine of CD4-Cre⁺ mice (WT).

Lachnospiraceae spps. Disclosed herein is a bacteria, Lachnospiraceae spps. Lachnospiraceae spps. as used herein refers to a bacteria having a 16S nucleic acid sequence sharing at least 98% sequence identity to SEQ ID NO: 4. Lachnospiraceae spps. has NRRL or ATCC Accession number ______. Lachnospiraceae spps. was isolated from a fecal pellet and luminal content from the lower the small intestine of CD4-Cre⁺ mice (WT).

Clostridia Consortium. The present disclosure is directed to compositions containing and methods of using bacterial isolates and communities. Disclosed herein is a Clostridia consortium (a mixture of two or more distinct strains of bacteria). In particular, the present disclosure is directed to compositions containing one or more microorganisms from the bacterial consortia as disclosed herein, particularly in Table 1 or mixtures thereof. In some aspects, the composition will include two or more bacterial strains from those listed in Table 1. The microorganisms can be characterized by an identifying 16S ribosomal gene sequence corresponding to, and at and least 98% identical to SEQ ID Nos: 1-4 and/or by comparison to bacteria with NRRL or ATCC Accession Nos: ______, ______, and ______, respectively. In some aspects, the Clostridia Consortium comprises two or more strains of bacterium having a 16S rDNA sequence comprising SEQ ID NOs: 1, 2, 3, and 4. In some aspects, the Clostridia Consortium comprises three or more strains of bacterium having a 16S rDNA sequence comprising SEQ ID NOs: 1, 2, 3, and 4. In some aspects, the Clostridia Consortium comprises fours strains of bacterium having a 16S rDNA sequence comprising SEQ ID NOs: 1, 2, 3, and 4, respectively.

In some aspects, the Clostridia Consortium comprises two or more strains of bacterium having a 16S rDNA sequence comprising SEQ ID NOs: 1, 2, 3, and 4 in the absence of any other strain of bacterium. In some aspects, the Clostridia Consortium comprises three or more strains of bacterium having a 16S rDNA sequence comprising SEQ ID NOs: 1, 2, 3, and 4 in the absence of any other strain of bacterium. In some aspects, the Clostridia Consortium comprises fours strains of bacterium having a 16S rDNA sequence comprising SEQ ID NOs: 1, 2, 3, and 4, respectively in the absence of any other strain of bacterium.

In some aspects, the Clostridia consortia comprises up to four of the bacterial strains listed in Table 1. In some aspects, the Clostridia consortia comprises two or more of the bacterial strains of Table 1. In some aspects, the Clostridia consortium comprises Clostridia anaerovorax, Clostridium XIVa, Clostridium IV and Lachnospiraceae spps. In some aspects, the Clostridia consortium comprises Clostridia anaerovorax, Clostridium XIVa, Clostridium IV, Lachnospiraceae spps and one or more of the bacterial strains of Table 1. In some aspects, the various bacteria in the consortia can be identified by their 16S ribosomal gene sequences.

In some aspects, the Clostridia consortium disclosed herein can reduce adiposity in a subject when colonized in the subject to the same degree as a complex microbial community that comprises a more complex Clostridia consortia that comprises more than the fours strains of bacterium having a 16S rDNA sequence comprising SEQ ID NOs: 1, 2, 3, and 4.

In some aspects, the Clostridia consortium disclosed herein can reduce weight gain and fat accumulation in WT mice or the obesity prone T-MyD88−/− mice when fed a high fat diet when compared to untreated WT mice or T-MyD88−/− mice, respectively.

In some aspects, the Clostridia consortium disclosed herein can lower body fat percentage and reduce VAT mass in WT mice or the obesity prone T-MyD88−/− mice when fed a high fat diet when compared to untreated WT mice or T-MyD88−/− mice, respectively.

In some aspects, the Clostridia consortium disclosed herein can decrease blood glucose levels and reduce insulin resistance in WT mice or the obesity prone T-MyD88−/− mice when fed a high fat diet when compared to untreated WT mice or T-MyD88−/− mice, respectively.

Disclosed herein is a Clostridia consortium for reducing adiposity in a subject, reducing weight gain and/or fat accumulation in a subject, lowering body fat percentage and/or reducing visceral adipose tissue (VAT) mass in a subject, decreasing blood glucose levels and/or reducing insulin resistance in a subject, inhibiting lipid absorption in a subject's small intestine, downregulating CD36 in a subject's liver and suppressing expression of lipid absorption genes within intestinal epithelia in a subject. In some aspects, the Clostridia consortia includes up to four of the bacterial strains listed in Table 1. In some aspects, a combination of any two or more of the bacterial strains of Table 1 can be used in a Clostridia consortia. In some aspects, the Clostridia consortium comprises Clostridia anaerovorax, Clostridium XIVa, Clostridium IV and Lachnospiraceae spps. In some aspects, the various bacteria in the consortia can be identified by their 16S ribosomal gene sequences.

TABLE 1
Bacterial Strains.

| | | SEQ ID |
|---|---|---|
| Name/Strain | 16S r Sequence | NO: |
| Clostridia anaerovorax | CTGCCCTTTGCACAGGGATAGCCATTGGAAACGATG | 1 |
| | ATTAAAACCTGATAACACCATTTGGTTACATGAGCA | |
| | GATGGTCAAAGATTTATCGGCAAAGGATGGGCCTGC | |
| | GTCTGATTAGCTAGTTGGTAAGGTAACGGCTTACCAA | |
| | GGCGACGATCAGTAGCCGACCTGAGAGGGTGAACGG | |
| | CCACATTGGAACTGAGACACGGTCCAAACTCCTACGG | |
| | GAGGCAGCAGTGGGGAATATTGCACAATGGGCGAAA | |
| | GCCTGATGCAGCAACGCCGCGTGAAGGAAGAAGGCC | |
| | TTCGGGTCGTAAACTTCTGTCCTTGGGGAAGAAGAA | |
| | CTGACGGTACCCAAGGAGGAAGCCCCGGCTAACTAC | |
| | GTGCCAGCAGCCGCGGTAATACGTAG | |
| Clostridium XIVa | CAGTTAGAAATGACTGCTAATACCGCATAAGACCAC | 2 |
| | AAAGCCGCATGGCTRWGTGGTAAAAACTCCGGTGGT | |
| | GTAAGATGGGCCCGCGTCTGATTAGGTAGTTGGCGG | |
| | GGTAACGGCCCACCAAGCCGACGATCAGTAGCCGAC | |
| | CTGAGAGGGTGACCGGCCACATTGGGACTGAGACAC | |
| | GGCCCAGACTCCTACGGGAGGCAGCAGTGGGGAATA | |
| | TTGCACAATGGGGGAAACCCTGATGCAGCGACGCCG | |
| | CGTGAGCGATGAAGTATTTCGGTATGTAAAGCTCTAT | |
| | CAGCAGGGAAGAAAATGACGGTACCTGACTAAGAAG | |
| | CCCCGGCTAACTACGTGCCAGCAGCCGCGGTAATACG | |
| | TAGGGGGCAAGCGTTA | |
| Clostridium IV | AGTTGGAAACGACTGCTAATACCGCATGATACATTTG | 3 |
| | GGTCGCATGGTCTGAATGTCAAAGATTTATCGCCGAA | |
| | AGATGGCCTCGCGTCTGATTAGCTAGTTGGTGGGGTA | |
| | ACGGCCCACCAAGGCGACGATC | |
| Lachnospiraceae spps. | TACAGGGGGATAACACTTAGAAATAGGTGCTAATACC | 4 |
| | GCATAAGCGCACAGGGGCGCATGCCCCGGTGTGAAAA | |
| | ACTCCGGTGGTATATGATGGACCCGCGTCTGATTAGCC | |
| | AGTTGGCAGGGTAACGGCCTACCAAAGCGACRATCAR | |
| | TAGCCGGCCTGAGAGGGCGGACGGCCACATTGGGACT | |
| | GAGACACGGCCCAA | |
| Anaerovorax | CAGGGATAGCCATTGGAAACGATGATTAAAACCTGAT | 5 |
| | AACACCATTTGGTTACATGAGCAGATGGTCAAAGATT | |
| | TATCGGCAAAGGATGGGCCTGCGTCTGATTAGCTAGTT | |
| | GGTAAGGTAACGGCTTACCAAGGCGACGATCAGTAGC | |
| | CGACCTGAGAGGGTGAACGGCCACATTGGAACTGAGA | |
| | CACGGTCCAAACTCCTACGGGAGGCAGCAGTGGGGAA | |
| | TATTGCACAATGGGCGAAAGCCTGATGCAGCAACGCC | |
| | GCGTGAAGGAAGAAGGCCTTCGGGTCGTAAACTTCTGT | |
| | CCTTGGGGAAGAAGAACTGACGGTACCCAAGGAGGAA | |
| | GCCCCGGCTAACTACGTGCCAGCAGCCGCGGTAATACG | |
| | TAGGGGGCAAGCGTTATCCGG | |
| Eisenbergiella | TACAGGGGGATAACACTTAGAAATAGGTGCTAATACCG | 6 |
| | CATAAGCGCACAGGGGCGCATGCCCCGGTGTGAAAAAC | |
| | TCCGGTGGTATATGATGGACCCGCGTCTGATTAGCCAGT | |
| | TGGCAGGGTAACGGCCTACCAAAGCGACRATCARTAGC | |
| | CGGCCTGAGAGGGCGGACGGCCACATTGGGACTGAGAC | |
| | ACGGCCCAA | |
| Anaerovorax | CATTGGAAACGATGATTAAAACCTGATAACACCATTT | 7 |
| | GGTTACATGAGCAGATGGTCAAAGATTTATCGGCAAA | |
| | GGATGGGCCTGCGTCTGATTAGCTAGTTGGTAAGGTA | |
| | ACGGCTTACCAAGGCGACGATCAGTAGCCGACCTGAG | |
| | AGGGTGAACGGCCACATTGGAACTGAGACACGGTCCA | |
| | AACTCCTACGGGAGGCAGCAGTGGGGAATATTGCACA | |
| | ATGGGCGAAAGCCTGATGCAGCAACSCCGCGTGAAGG | |
| | AAGAAGGCCTTCGGGTCGTA | |
| Hungatella | GGGGGACAACAGTTAGAAATGACTGCTAATACCGCAT | 8 |
| | AAGCGCACGGGAACGCATGTTTCTGTGTGAAAAACTC | |
| | CGGTGGTGTAAGATGGGCCCGCGTTGGATTAGGT | |
| Anaerotruncus | CCATTGGAAACGATGATTAAAACCTGATAACACCATT | 9 |
| | TGGTTACATGAGCAGATGGTCAAAGATTTATCGGCAA | |
| | AGGATGGGCCTGCGTCTGATTAGCTAKTTGGTAAGGT | |
| | AACGGCTTACCAAGGCGACGATCAGTAGCCKACCTGAG | |
| Anaerotruncus | TAATACCGCATGAGACTACAGTACTACATGGTACAG | 10 |
| | TGGCCAAAGGAGCAATCCGCTGAAAGATGGGCTCG | |
| | CGTCCGATTAGATAGTTGGCGGGGTAACGGCCCACC | |
| | AAGTCGACGATCGGTAGCCGGACTGAGAGGTTGAA | |
| | CGGCCACRTTGGGACTGAGACACGGCCCAGACTCCT | |
| | ACGGGAGGCAGCAGTGAGGGATATTGGTCAATGGGG | |
| Anaerovorax | AGCCATTGGAAACGATGATTAAAACCTGATAACAC | 11 |
| | CATTTGGTTACATGAGCAGATGGTCAAAGATTTATC | |
| | GGCAAAGGATGGGCCTGCGTCTGATTAGCTAGTTGG | |
| | TAAGGTAACGGCTTACCAAGGCGACGATCAGTAGC | |
| | CGACCTGAGAGGGTGAACGGCCACATTGGAACTGA | |
| | GACACGGTCCAAACTCCTACGGGAGGCAGCAGTGG | |
| | GGAATATTGCACAATGGGCGAAAGCCTGATGCAGC | |
| | AACGCCGCGTGAAGGAAGAAGGCCTTCGGGTCGTA | |
| | AACTTCTGT | |
| Anaerovorax | GTAGGCAACCTGCCCTTWGCACAGGGATAGCCATT | 12 |
| | GGAAACGATGATTAAAACCTGATAACACCATTTGG | |
| | TTACATGAGCAGATGGTCAAAGATTTATCGGCAAA | |
| | GGATGGGCCTGCGTCTGATTAGCTAGTTGGTAAGGT | |
| | AACGGCTTACCAAGGCGACGATCAGTAGCCGACCTG | |
| | AGAGGGTGAACGGCCACATTGGAACTGAGACACGG | |
| | TCCAAACTCCTACGGGAGGCAGCAGTGGGGAATATT | |
| | GCACAATGGGCGAAAGCCTGATGCAGCAACGCCGC | |
| | GTGAAGGAAGAAGGCCTTCGGGTCGTAAACTTCTG | |
| | TCCTTGGGGAAGAAGAACTGACGGTACCCAAGGAG | |
| | GAAGCCCCGGCTAACTACGTGCCAGCAGCCGCGGT | |
| | AATACGTAGGGGGCAAG | |
| Streptococcus | GCTAATACCGCATAAGAGTAGATGTTGCATGACATT | 13 |
| | TGCTTAAAAGGTGCAATTGCATCACTACCAGATGGA | |
| | CCTGCGTTGTATTAGCTAGTTGGTGGGGTAACGGCT | |
| | CACCAAGGCGACGATACATAGCCGACCTGAGAGGG | |
| | TGATCGGCCACACTGGGACTGAGACACG | |
| Streptococcus | ATACCGCATAAGAGTAGATGTTGCATGACATTTGCT | 14 |
| | TAAAAGGTGCAATTGCATCACTACCAGATGGACCT | |
| | GCGTTGTATTAGCTAGTTGGTGGGGTAACGGCTCA | |
| | CCAASGCGACGATACATAGCCGACCTGAGAGGGTG | |
| | ATCGGCCRCACTGGGACCGAGAC | |
| Dehalobacter | CCCCATAGAGGGGGACAACAGCTGGAAACGGCTG | 15 |
| | CTAATACCGCATAGCAGGAAAGAGACGCATGTCT | |
| | TTTTCTTCAAAGATTTATCGCTATGGGATGGACCC | |
| | GCGTCTGATTAGCTAGTTGGTAAGGTAACGGCCT | |
| | ACCAA | |
| Ruminococcus2 | AACTCCTACGGGAGGCAGCAGTGGGGAATATTGCA | 16 |
| | CAATGGGCGCAAGCCTGATGCAGCGACSCCGCGTG | |
| | AGCGAAGAAGTATTTCGGTATGTAAAGCTCTATCA | |
| | GCAGGGAAGAAAATGACRGTACCTGACTAAAAAG | |
| | CTCCGGCTAAATACRTGYCAGCASCCSCGGTAATAC | |
| | GTATGGAGCAAGCGTTATCCGGAATTACTGKGTGTA | |
| | AAGGGAGCGTATACGGATGTGCAAGTCTGATGTGAA | |
| | AGGCG | |
| Anaerovorax | GGGGCAAGCGTTATCCGGAATTATTGGGCGTAAAAG | 17 |
| | AGTACGTAGGTGGCAACCTAAGCGCAGGGTTTAAGG | |
| | CAATGGCTCAACCATTGTTCGCCCCTGCGAACTGGAG | |
| | AATGCTTGAGTGCAGGAGAGGAAAAGCGGAATTCCT | |
| | AGTGTAGCGGTGAAAATGCGTAGATATTAGGAGGAA | |
| | CACCAGTGGCGAAGGCGGCTTTCTGGACTGTAACTGA | |
| | CACTGAGGTACGAAAGCGTGGGGAGCAAACAGGATT | |
| | AGATACCCTGGTAGTCCACGCCGTAAACGATGAGCAC | |
| | TAGGTGTCGGGGTCGCAAGACTTCGGTGCCGTAGTTA | |
| | ACGCATTAAGTGCTCCGCCTGGGGGAGTACGCACGCA | |
| | AGTGTGAAACTCAAAGGAAATTGACGGGGGACCCCG | |
| | CACAAGCAGCGGAGCATGTGGTTTAATTCGAAGCAA | |
| | CGCGAAAGAAACCTTACCAGGACTTGACATCCCTCT | |
| | GACAGACCCTT | |
| Lachnospiracea_incertae_sedis | CTGATGCAGCGACGCCGCGTGAGCGAAGAAGTATTT | 18 |
| | CGGTATGTAAAGCTCTATCAGCAGGGAAGAAAATGA | |
| | CGGTACCTGAGTAAGAAGCTCCGGCTAAATACGTGC | |
| | CAGCAGCCGCGGTAATACGTATGGAGCAAGCGTTAT | |
| | CCGGATTTACTGGGTGTAAAGGGAGCGCAGGCGGCA | |
| | GGGCAAGTCTGATGTGAAATACCGGGGCTCAACCCC | |
| | GGAGCTGCATTGGAAACTGTTCTGCTGGAGTGTCGG | |
| | AGAGGCAGGCGGAATTCCTAGTGTAGCGGTGAAATG | |
| | CGTAGATATTAGGAGGAACACCAGTGGCGAAGGCGG | |
| | CCTGCTGGACGATAACTGACGCTGAGGCTCGAAAGCG | |
| | TGGGGAGCAAACAGGATTAGATACCCTGGTAGTCCAC | |
| | GCCGTAAACGATGAATACTAGGTGTCGGGGAGCAAA | |
| | GCTCTTCGGTGCCGCAGCAAACGCAGTAAGTATTCCA | |
| | CCTGGGGAGTACGTTCGCAAGAATGAAACTCAAAGG | |
| | AATTGACGGGGACCCGCACAAGCGGTGGAGCATGTG | |
| | GTTTAATTCGAAGCAACGCGAAGAACCTTACCAAGCC | |
| | TTGACATCCCGATGACAGCATATGTAATGTATGTTCCC | |
| | TTTTTGGGCATTGGAGACAGGTGGTGCATGGTTGTCG | |
| | TCAGCTCGTGTCGTGAGATGTTGGGTTAAGTCCCGCA | |
| | ACGAGCGCAACCCTTATCCTTAGTAGCCAGCAGGCAG | |
| Butyrivibrio | ATGTAAAGCTCTATCAGCAGGGAAGAAAATGACGGT | 19 |
| | ACCTGACTAAGAAGCCCCGGCTAACTACGTGCCAGC | |
| | AGCCGCGGTAATACGTAGGGGGCAAGCGTTATCCGG | |
| | ATTTACTGGGTGTAAAGGGAGCGTAGACGGCAGCGC | |
| | AAGTCTGAAGTGAAATCCCATGGCTTAACCATGGAA | |
| | CTGCTTTGGAAACTGTGCAGCTGGAGTGCAGGAGAG | |
| | GTAAGCGGAATTCCTAGTGTAGCGGTGAAATGCGTA | |
| | KATATTAGGAGGAACACCAGTGGCGAAGGCGGCTT | |
| | ACTGGACTGTAACTGACGTTGAGGCTCGAAAGCGT | |
| | GGGGAGCAAACAGGATTAGATACCCTGGTAGTCCA | |
| | CGCCSTAAACGATGATTACTAGGTGTTGGGGGACCA | |
| | AGGTCCTTCGGTGCCGGCGCAAACGCATTAAGTAAT | |
| | CCACCTGGGGAGTACGTTC | |
| Anaerovorax | TAACTACGTGCCAGCAGCCGCGGTTAATACGTAGGG | 20 |
| | GGGGCAAGCGTTATCCGGAATTATTGGGCGTAAAG | |
| | AGTACGTAGGTGGCAACCCTAAGCGCAGGGGTTTT | |
| | AAGGCAATGGCTCAACCATTGTTCGCCCTGCGACT | |
| | GGGATGCTTGAGTGCAGGAGAGGAAAAGCGGAAT | |
| | TCCTAGTGTAGCGGTGAAAATGCGTAGATATTAGG | |
| | AGGAACACCAGTGGCGAAGGCGGCTTTCTGGACTG | |
| | TAACTGACACTGAGGTACGAAAGCGTGGGGAGCAA | |
| | ACAGGATTAGATACCCTGGTAGTCCACGCCGTAAAC | |
| | GATGAGCACTAGGTGTCGGGGTCGCAAGACTTCGGT | |
| | GCCGTAGTTAACGCATTAAGTGCTCCGCCTGGGGAG | |
| | TACGCACGCAAGTGTGAAACTCAAAGGAATTGACG | |
| | GGGACCCGCACAAGCAGCGGAGCATGTGGTTTAATT | |
| | CGAAGCAACGCGAAGAACCTTACCAGGACTTGACAT | |
| | CCCTCTGACAGACCCTTAATCGGGTTTTTCTACGGAC | |
| | AGAGGAACAGGTGGTGCATGGGTTGTCGTCAGCTC | |
| | GTGTCGTGAGATGTTGGGTTAAGTCCCGCAACGAG | |
| | CGCAACTCTTTGCCATTAGTTTGCCAGCAGTAAGAT | |
| | GGGCACTCTAGTGGGACTGCC | |
| Anaerovorax | AAATGCGTAGATATTAGGAGGAACACCAGTGGCG | 21 |
| | AAGGCGGCTTTCTGGACTGTAACTGACACTGAGGT | |
| | ACGAAAGCGTGGGGGAGCAAACAGGATTAGATA | |
| | CCCTGGTAGTCCACGCCGTAAACGATGAGCACTA | |
| | GGTGTCGGGGTCGCAAGACTTCGGTGCCGTAGTT | |
| | AACGCATTAAGTGCTCCGCCTGGGGGAGTACGCA | |
| | CGCAAGTGTGAAACTCAAAGGAATTGACGGGGA | |
| | CCCGCACAAGCAGCGGAGCATGTGGTTTAATTCGA | |
| | AGCAACGCGAAGAAACCTTACCAGGACTTGACAT | |
| | CCCTCTGACAGACCCTTAATCGGGTTTTTTTCTAC | |
| | GGACAGAGGAGACAGGTGGTGCATGGTTGTCGT | |
| | CAGCTCGTGTCGTGAGATGTTGGG | |
| Anaerovorax | ACGTAGGGGGCAAGCGTTATCCCGGAATTATTGGG | 22 |
| | CGTAAAGAGTACGTAGGTGGCAACCTAAGCGCAGG | |
| | GGTTTAAGGCAATGGCTCAACCATTGTTCGCCCTGC | |
| | GAACTGGGATGCTTGAGTGCAGGAGAGGAAAGCG | |
| | GAATTCCTAGTGTAGCGGTGAAATGCGTAGATATT | |
| | AGGAGGAACACCAGTGGCGAAGGCGGCTTTTCTG | |
| | GACTGTAACTGACACTGAGGTACGAAAAGCGTGG | |
| | GGGAGCAAACAGGATTAGATACCCCTGGTAGTCC | |
| | ACGCCGTAAACGATGAGCACTAGGTGTCGGGGGT | |
| | CGCAAGACTTCGGTGCCGTAGTTAACGCATTAAG | |
| | TGCCTCCGCCTGGGGGAGTACGCACGCCAAGTGT | |
| | GAAACTCATAGGAATTGACGGGGACCCGCACAAG | |
| | CAGCG | |
| Eubacterium | GAGACACGGTCCAAACTCCTACGGGAGGCAGCAG | 23 |
| | TGGGGAATATTGCACAATGGGCGAAAGCCTGATG | |
| | CAGCAACGCCGCGTGAAGGAAGAAGGCCTTCGGG | |
| | TCGTAAACTTCTGTCCTTGGGGAAGAAGAACTGAC | |
| | GGTACCCAAGGAGGAAGCCCCGGCTAACTACGTG | |
| | CCAGCAGCCGCGGTAATACGTAGGGGGCAAGCGT | |
| | TATCCGGAATTATTGGGCGTAAAGAGTACGTAGGT | |
| | GGCAACCTAAGCGCAGGGTTTAAGGCAATGGCTCA | |
| | ACCATTGTTCGCCCTGCGAACTGGGATGCTTGAGTG | |
| | CAGGAGAGGAAAGCGGAATTCCTAGTGTAGCGGTG | |
| | AAATGCGTAGATATTAGGAGGAACACCAGTGGCGA | |
| | AGGCGGCTTTCTGGACTGTAACTGACACTGAGGTAC | |
| | GAAAGCGTGGGGAGCAAACAGGATTAGATACCCTG | |
| | GTAGTCCACGCCGTAAACGATGAGCACTAGGTGTC | |
| | GGGGTCGCAAGACTTCGGTGCCGTAGTTAACGCAT | |
| | TAAGTGCTCCGCCTGGGGAGTACGCACGCAAGTGT | |
| | GAAACTCAAAGGAATTGACGGGGACCCGCACAAG | |
| | CAGCGGAGCATGTGGTTTAATTCGAAGCAACGCGA | |
| | AGAACCTTACCAGGACTTGACATCCCTCTGACAGA | |
| | CCCTTAATCGGGTTTTTCTACGGACAGAGGAGACA | |
| | GGTGGTGCATGGTTGTCGTCAGCTCGTGTCGTGAG | |
| | ATGTTGGGTTAAGTCCCGCAACGAGCGCAACCCTT | |
| | GCCATTAGTTGCCAGCAGTAAGATGGGCACTCTAG | |
| | TGGGACTGCCGGGGACAACTCGGAGGAAGGTGGG | |
| | GATGACGTCAAATCATCATGCCCCTTATGTTCTGG | |
| | GCTACACACGTGCTACAATGGCCGGTACA | |
| Anaerovorax | GAGCGAGAAGCTGATGACAGATACTTCGGTTGAAG | 24 |
| | GAGTCAGTGGAAAGCGGCGGACGGGTGAGTAACGC | |
| | GTAGGCAACCTGCCCTTTGCACAGGGATAGCCATTG | |
| | GAAACGATGATTAAAACCTGATAACACCATTTGGTT | |
| | ACATGAGCAGATGGTCAAAGATTTATCGGCAAAGG | |
| | ATGGGCCTGCGTCTGATTAGCTAGTTGGTAAGGTAA | |
| | CGGCTTACCAAGGCGACGATCAGTAGCCGACCTGA | |
| | GAGGGTGAACGGCCACATTGGAACTGAGACACGGT | |
| | CCAAACTCCTACGGGAGGCAGCAGTGGGGAATATT | |
| | GCACAATGGGCGAAAGCCTGATGCAGCAACGCCGC | |
| | GTGAAGGAAGAAGGCCTTCGGGTCGTAAACTTCTG | |
| | TCCTTGGGGAAGAAGAACTGACGGTACCCAAGGAG | |
| | GAAGCCCCGGCTAACTACGTGCCAGCAGCCGCGGT | |
| | AATACGTAGGGGGCAAGCGTTATCCGGAATTATTG | |
| | GGCGTAAAGAGTACGTAGGTGGCAACCTAAGCGCA | |
| | GGGTTTAAGGCAATGGCTCAACCATTGTTCGCCCTG | |
| | CGAACTGGGATGCTTGAGTGCAGGAGAGGAAAGCG | |
| | GAATTCCTAGTGTAGCGGTGAAATGCGTAGATATT | |
| | AGGAGGAACACCAGTGGCGAAGGCGGCTTTCTGG | |
| | ACTGTAACTGA | |
| Anaerovorax | AGCGAGAAGCTGATGACAGATACTTCGGTTGAAG | 25 |
| | GAGTCAGTGGAAAGCGGCGGACGGGTGAGTAAC | |
| | GCGTAGGCAACCTGCCCTTTGCACAGGGATAGCC | |
| | ATTGGAAACGATGATTAAAACCTGATAACACCAT | |
| | TTGGTTACATGAGCAGATGGTCAAAGATTTATCG | |
| | GCAAAGGATGGGCCTGCGTCTGATTAGCTAGTTG | |
| | GTAAGGTAACGGCTTACCAAGGCGACGATCAGTA | |
| | GCCGACCTGAGAGGGTGAACGGCCACATTGGAAC | |
| | TGAGACACGGTCCAAACTCCTACGGGAGGCAGCA | |
| | GTGGGGAATATTGCACAATGGGCGAAAGCCTGAT | |
| | GCAGCAACGCCGCGTGAAGGAAGAAGGCCTTCGG | |
| | GTCGTAAACTTCTGTCCTTGGGGAAGAAGAACTG | |
| | ACGGTACCCAAGGAGGAAGCCCCGGCTAACTAC | |
| | GTGCCAGCAGCCGCGGTAATACGTAGGGGGCAA | |
| | GCGTTATCCGGAATTATTGGGCGTAAAGAGTAC | |
| | GTAGGTGGCAACCTAAGCGCAGGGTTTAAGGC | |
| | AATGGCTCAACCATTGTTCGCCCTGCGAACTGG | |
| | GATGCTTGAGTG | |
| Clostridium XIVa | TCGAACGAAGCGATTTAACGGAAGTTTTCGGAT | 26 |
| | GGAAGTTGAATTGACTGAGTGGCGGACGGGTG | |
| | AGTAACGCGTGGGTAACCTGCCTTGTACTGGGG | |
| | GACAACAGTTAGAAATGACTGCTAATACCGCAT | |
| | AAGCGCACAGTATCGCATGATACAGTGTGAAAA | |
| | ACTCCGGTGGTACAAGATGGACCCGCGTCTGATT | |
| | AGCTAGTTGGTAAGGTAACGGCTTACCAAGGCGA | |
| | CGATCAGTAGCCGACCTGAGAGGGTGACCGGCCA | |
| | CATTGGGACTGAGACACGGCCCAAACTCCTACGG | |
| | GAGGCAGCAGTGGGGAATATTGCACAATGGGCGA | |
| | AAGCCTGATGCAGCGACGCCGCGTGAGTGAAGAA | |
| | GTATTTCGGTATGTAAAGCTCTATCAGCAGGGAAG | |
| | AAAATGACGGTACCTGACTAAGAAGCCCCGGCTAA | |
| | CTACGTGCCAGCAGCCGCGGTAATACGTAGGGGGC | |
| | AAGCGTTATCCGGATTTACTGGGTGTAAAGGGAG | |
| | CGTAGACGGTAAAGCAAGTCTGAAGTGAAAGCCC | |
| | GCGGCTCAACTGCGGGACTGCTTTGGAAACTGTTT | |
| | AACTGGAGTGTCGGAGAGGTAAGTGGAATTCCTA | |
| | GTGTAGCGGTGAAATGCGTAGATATTAGGAGGAA | |
| | CACCAGTG | |
| Lactobacillus | AAGTGCGTGAGAGTAACTGTTCACGTTTCGACGGT | 27 |
| | ATCTAACCAGAAAGCCACGGCTAACTACGTGCCAG | |
| | CAGCCGCGGTAATACGTAGGTGGCAAGCGTTATCC | |
| | GGATTTATTGGGCGTAAAGGGAACGCAGGCGGTCT | |
| | TTTAAGTCTGATGTGAAAGCCTTCGGCTTAACCGG | |
| | AGTAGTGCATTGGAAACTGGGAGACTTGAGTGCAG | |
| | AAGAGGAGAGTGGAACTCCATGTGTAGCGGTGAA | |
| | ATGCGTAGATATATGGAAGAACACCAGTGGCGAA | |
| | AGCGGCTCTCTGGTCTGTAACTGACGCTGAGGTTC | |
| | GAAAGCGTGGGTAGCAAACAGGATTAGATACCCT | |
| | GGTAGTCCCGCCGTAAACGATGAATGCTAAGTGTT | |
| | GGAGGGTTTCCGCCCTTCAGTGCTGCAGCTAACGC | |
| | AATAAGCATTCCGCCTGGGGAGTACGACCGCAAG | |
| | GTTGAAACTCAAAGGAATTGACGGGGGGCCCGCA | |
| | CAAGCGGTGGAGCATGTGGTTTAATTCGAAGCAA | |
| | CGCGAAGAACCCTTACCAAGGTCTTGACATCTTTT | |
| | TGACAATCCCTAGAGATAGGACTTTCCCTTCGGGG | |
| | ACAAAATGACAGGTGGTGCATG | |
| Clostridium IV | GACTCCTACGGGAGGCAGCAGTGAGGGATATTGG | 28 |
| | TCAATGGGGGAAACCCTGAACCAGCAACGCCGCG | |
| | TGAGGGAAGACGGTTTTCGGATTGTAAACCTCTGT | |
| | CCTCTGTGAAGATAATGACGGTAGCAGAGGAGGA | |
| | AGCTCCGGCTAACTACGTGCCAGCAGCCGCGGTA | |
| | ATACGTAGGGAGCAAGCGTTGTCCGGATTTACTG | |
| | GGTGTAAAGGGTGCGTAGGCGGCCTTGCAAGTCA | |
| | GAAGTGAAATCCATGGGCTTAACCCGTGAACTGC | |
| | TTTTGAAACTGTAGGGCTTGAGTGAAGTAGAGGC | |
| | AGGCGGAATTCCCGGTGTAGCGGTGAAATGCGTA | |
| | GAGATCGGGAGGAACACCAGTGGCGAAGGCGGC | |
| | CTGCTGGGCTTTAACTGACGCTGAAGCACGAAAG | |
| | CGTGGGTAGCAAACAGGATTAGATACCCTGGTAG | |
| | TCCACGCCGTAAACGATGATTACTAGGTGTGGGG | |
| | GGGGTCTGACCCCCCTCCGTGCCGGAGTTAACAC | |
| | AATAAGTAATCCACCTGGGGGAGTACGGCCGCAA | |
| | GGCTGAAACTCAAAGGAAATTGACGGGGGGCCCG | |
| | CACAAGCAGTGGAGTATGTGGATTAATTCGAAGCC | |
| | AACGCGAAGAACCTTACCAGGTCTTGACATCCCCG | |
| | GCGACCGGCTTAGAGATA | |
| Clostridium IV | CACGGCCCAGACTCCTACGGGAGGCAGCAGTGGG | 29 |
| | GAATATTGCACAATGGGGGAAACCCTGATGCAGC | |
| | GACGCCGCGTGAGCGATGAAGTATTTCGGTATGTA | |
| | AAGCTCTATCAGCAGGGAAGAAAATGACGGTACC | |
| | TGACTAAGAAGCCCCGGCTAACTACGTGCCAGCA | |
| | GCCGCGGTAATACGTAGGGGGCAAGCGTTATCCG | |
| | GATTTACTGGGTGTAAAGGGAGCGTAGACGGCGG | |
| | TGCAAGCCAGATGTGAAAGCCCGGGGCTCAACCC | |
| | CGGGACTGCATTTGGAACTGTGCTGCTAGAGTGTC | |
| | GGAGAGGCAGGCGGAATTCCTAGTGTAGCGGTGA | |
| | AATGCGTAGATATTAGGAGGAACACCAGTGGCGA | |
| | AGGCGGCCTGCTGGAGATGACTGACGTTGAGGCT | |
| | CGAAAGCGTGGGGAGCAAACAGGATTAGATACC | |
| | CTGGTAGTCCACGCCGTAAACGATGACTACTAGG | |
| | TGTCGGGCAGCAAAGCTGTTCGGTGCCGCAGCCA | |
| | ACGCAATAAGTAGTCCACCTGGGGAGTACGTTCG | |
| | CAAGAATGAAACTCAAAGGAATTGACGGGGACC | |
| | CGCACAAGCGGTGGAGCATGTGGTTTAATTCGAA | |
| | GCAACGCGAAGAACCTTACCTGGYCTTGACATCC | |
| | CCCCTGACCGGCTCGTAATGGGGCCTTTCCTTCGG | |
| | GACAAGGGGGAGAACAGGTGGTGCATGGATTGTC | |
| | GTCAGCTCGTGTCGTGAGATGTTGG | |
| Olsenella | AAGTCGAACGGGAAGCGGGGCCTCCGGGCCCCG | 30 |
| | CCGAGAGTGGCGAACGGCTGAGTAACACGTGGG | |
| | CAACCTGCCCCCTCCACCGGGACAGCCTCGGGAA | |
| | ACCGTGGGTAATACCGGATACTCCGGGACGGCCG | |
| | CATGGCCGGCCCGGGAAAGCCCAGACGGGAGGG | |
| | GATGGGCCCGCGGCCTGTTAGCTAGTCGGCGGGG | |
| | TAACGGCCCACCGAGGCGATTATGGGTAGCCGGG | |
| | TTGAGAGACCGACCAGCCAGATTG | |
| Marvinbryantia | AAATGCGTAGATATCAGGAGGAACACCAGTGGCG | 31 |
| | AAGGCGGCCTGCTGGACGATGACTGACGCTGAGG | |
| | CTCGAAAGCGTGGGGAGCAAACAGGATTAGATAC | |
| | CCTGGTAGTCCACGCCGTAAACGATGAATACCAG | |
| | GTGTCGGGGAGCAGGGCTCTTCGGTGCCGCAGCA | |
| | AACGCAGTAAGTATTCCACCTGGGGAGTACGTTC | |
| | GCAAGAATGAAACTCAAARGGAATTGACGGGGAC | |
| | CCGCACAAGCGGTGGAGCATGTGGTTTAATTCGAA | |
| | GCAACGCGAAGACCCTTACTCAGGCCTTGACATC | |
| | CCGGGTGACAGCATATGTAATGTATGTTCCCTTC | |
| | GGGGCA | |
| Coprococcus | GCTCACCAAGGCGACGATCAGTAGCCGGCCTGAG | 32 |
| | AGGGTGAACGGCCACATTGGGACTGAGACACGGC | |
| | CGAAACTCCTACGGGAGGCAGCAGTGGGGAATAT | |
| | TGSASAATGGGGGAAACCCTGATGCAGCGACGCCG | |
| | CGTGAAGGAAGAAGTATTTCGGTATGTAAACTTCT | |
| | ATCAGCAGGGAAGAAAATGACGGTACCTGACTAA | |
| | GAAGCCCCGGCTAACTACGTGCCAGCAGCCGCGG | |
| | TAATACGTAGGGGGCAAGCGTTATCCGGATTTACT | |
| | GGGTGTAAAGGGAGCGTAGGCGGTTCAGCAAGTC | |
| | AGAAGTGAAAGCCCGGGGCTCAACTCCGGGACTG | |
| | CTTTTGAAACTGTTGAACTAGATTGCAGGAGAGGT | |
| | AAGTGGAATTCCTAGTGTAGCGGTGAAATGCGTA | |
| | GATATTAGGAGGAACACCAGTGGCGAAAGCGGCT | |
| | TACTGGACTGTAAATGACGCTGAGGCTCGAAAGC | |
| | GTGRGGGAGCAAACAGGATTAGATACCCTGGTAG | |
| | TCCACGCCGTAAACGATGAATACTAGGTGTCAGG | |
| | CGCCATAGGCGTTTGGTGCCGCAGCAAACGCAAT | |
| | AAGTATTCCACCTGGAGGAAGTACGTTCGCAAGA | |
| | ATGAAACTCAAAGGAATT | |

Disclosed herein are composition comprising a supernatant from a Clostridia consortium. Also disclosed herein are compositions comprising a Clostridium consortium. In some aspects, the Clostridia consortium comprises Clostridia anaerovorax, Clostridium XIVa, Clostridium IV and Lachnospiraceae spps. In some aspects, the Clostridia consortium comprises one or more of Clostridia anaerovorax, Clostridium XIVa, Clostridium IV and Lachnospiraceae spps. In some aspects, the Clostridia consortium comprises two or more of Clostridia anaerovorax, Clostridium XIVa, Clostridium IV and Lachnospiraceae spps. In some aspects, the Clostridia consortium comprises three or more of Clostridia anaerovorax, Clostridium XIVa, Clostridium IV and Lachnospiraceae spps. In some aspects, the Clostridia consortium consists of Clostridia anaerovorax, Clostridium XIVa, Clostridium IV and Lachnospiraceae spps. In some aspects, the Clostridia consortium consists of one or more of Clostridia anaerovorax, Clostridium XIVa, Clostridium IV and Lachnospiraceae spps. In some aspects, the Clostridia consortium consists of two or more of Clostridia anaerovorax, Clostridium XIVa, Clostridium IV and Lachnospiraceae spps. In some aspects, the Clostridia consortium consists of three or more of Clostridia anaerovorax, Clostridium XIVa, Clostridium IV and Lachnospiraceae spps.

In some aspects, any of the compositions described herein is capable of suppressing expression of lipid adsorption genes within intestinal epithelia in a subject. In some aspects, any of the compositions disclosed herein can suppress one or more lipid absorption and/or synthesis genes. In some aspects, the lipid absorption genes can be CD36, FasN, Dgat, Srepbf1, SLC27a1, and SLC27a4.

In some aspects, any of the compositions described herein is capable of inhibiting lipid absorption in a subject's small intestine.

In some aspects, any of the compositions described herein is capable of reducing weight gain in a subject.

In some aspects, any of the compositions described herein is capable of downregulating CD36 in a subject's liver.

Also, disclosed herein are a consortium of bacteria comprising two or more Clostridia anaerovorax strain, Clostridium XIVa, Clostridium IV, and Lachnospiraceae spps, wherein the consortium suppresses expression of lipid adsorption genes within intestinal epithelia in a subject compared to a subject where the consortium has not been administered.

In some aspects, the compositions described herein can further comprise one or more Clostridia strains selected from Table 1.

In some aspects, the disclosed compositions include at least two or more bacterial microorganisms identifiable by homology of at least 95, 96, 97, 98, 99 or greater percent identity to the 16S ribosomal sequences of SEQ ID NOs: 1-4. In some aspects, the amount of 16S sequence is less than about 1.2 kb, 1.1 kb, 1.0 kb, 0.9 kb, 8 kb, 0.7 kb, 0.6 kb, 0.5 kb, 0.4 kb, 0.3 kb, 0.2 kb, or 0.1 kb and greater than about 50 nt, 0.1 kb, 2 kb, 0.3 kb, 0.4 kb, 0.5 kb, 0.6 kb, 0.7 kb, 0.8 kb, 0.9 kb, 1.0 kb, or 1.1 kb. In some aspects, the amount of 16S ribosomal sequence homology is between about 150 nt and 500 nt, for example about 250 nt. To determine the percent identity of two nucleic acids, the sequences are aligned for optimal comparison purposes (e.g., gaps can be introduced in the sequence of a first nucleic acid sequence for optimal alignment with a second nucleic acid sequence). The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences (i.e., % identity=# of identical positions/total # of positions times 100).

The determination of percent homology between two sequences may be accomplished using a mathematical algorithm. A preferred, non-limiting example of a mathematical algorithm utilized for the comparison of two sequences is the algorithm of Karlin and Altschul (1990) Proc. Nat'l Acad. Sci. USA 87:2264-2268, modified as in Karlin and Altschul (1993) Proc. Nat'l Acad. Sci. USA 90:5873-5877. Such an algorithm is incorporated into the NBLAST and)(BLAST programs of Altschul, et al. (1990) J. Mol . Biol. 215:403-410. BLAST nucleotide searches can be performed with the NBLAST program, score=100, word length=12 to obtain nucleotide sequences similar or homologous to nucleic acid molecules of the present disclosure. To obtain gapped alignments for comparison purposes, Gapped BLAST can be utilized as described in Altschul et al. (1997) Nucleic Acids Res. 25:3389-3402. When utilizing BLAST and Gapped BLAST programs, the default parameters of the respective programs (e.g., XBLAST and NBLAST) can be used. These algorithms may be used to align DNA with RNA, and in some cases may be used to align proteins with translated nucleotide sequences.

In some aspects, at least two or more microorganisms are included in the compositions of the present disclosure. It is contemplated that where two or more microorganisms form the composition, the microorganisms may be co-cultured to produce the disclosed composition. In some aspects, the disclosed composition may be formed by combining individual cultures of the two or more strains. The microorganisms may be propagated by methods known in the art. For example, the microorganisms may be propagated in a liquid medium under anaerobic or aerobic conditions. Suitable liquid mediums used for growing microorganism include those known in the art such as Nutrient Broth and Tryptic soy agar (TSA), etc. In some aspects, the composition includes the entire listing of the strains listed in Table 1. In some aspects, the composition includes at least two or more of the following strains: Clostridia anaerovorax, Clostridium XIVa, Clostridium IV, and Lachnospiraceae spps.

In some aspects, the compositions disclosed herein can comprise at least 1×10⁻⁵ cells of each Clostridia strain. In some aspects, the compositions disclosed herein can comprise at least 1×10⁻⁶ cells of each Clostridia strain. In some aspects, the compositions disclosed herein can comprise at least 1×10⁻⁷ cells of each Clostridia strain. In some aspects, the compositions disclosed herein can comprise at least 1×10⁻⁸ cells of each Clostridia strain. In some aspects, the compositions disclosed herein can comprise at least 1×10⁻⁹ cells of each Clostridia strain. In some aspects, the compositions disclosed herein can comprise at least 1×10⁻¹⁰ cells of each Clostridia strain. In some aspects, a single dosage of any of the compositions disclosed herein can comprise between 1×10⁻⁵ and 1×10⁻¹⁰ cells of each Clostridia strain. In some aspects, the cells of the consortia are active.

In some aspects, the compositions disclosed herein are capable of replacing microbiota of a subject with a disease or disorder associated with an imbalanced microbiota. In some aspects, the compositions disclosed herein are capable of replacing microbiota of a subject with a disease or disorder associated with a dysfunctional microbiota. In some aspects, the compositions disclosed herein are capable of replacing microbiota of a subject with a disease or disorder associated with microbiota that is decreased in functional diversity. In some aspects, the imbalanced microbiota (or dysfunctional microbiota or a microbiota that is decreased in functional diversity) can be an increase in Desulfovibrio and decrease of Clostridia. In some aspects, the imbalanced microbiota (or dysfunctional microbiota or a microbiota that is decreased in functional diversity) can be no change in (or no expansion of) Desulfovibrio and decrease of Clostridia. In some aspects, the imbalanced microbiota (or dysfunctional microbiota or a microbiota that is decreased in functional diversity) can be a decrease of Clostridia. In some aspects, the imbalanced microbiota (or dysfunctional microbiota or a microbiota that is decreased in functional diversity) can be an absence or lack of Clostridia.

In some aspects, the disease or disorder can be obesity, metabolic syndrome, insulin deficiency, insulin-resistance related disorders, glucose intolerance, diabetes, or an inflammatory bowel disease. In some aspects, the inflammatory bowel disease can be Crohn's disease or ulcerative colitis. In some aspects, insulin-resistance related disorder can be diabetes, hypertension, dyslipidemia, or cardiovascular disease. In some aspects, diabetes can be Type I diabetes. In some aspects, diabetes can be Type II diabetes.

In some aspects, the compositions disclosed herein can further comprising a pharmaceutically acceptable carrier. In some aspects, the compositions may also include additives. Suitable additives include substances known in the art that may support growth, production of specific metabolites by the microorganism, alter pH, enrich for target metabolites, enhance insecticidal effects, and combinations thereof. Exemplary additives include carbon sources, nitrogen sources, phosphorous sources, inorganic salt, organic acid, growth media, vitamins, minerals, acetic acid, amino acids and the like.

Examples of suitable carbon sources include, without limitation, starch, peptone, yeast extract, amino acids, sugars such as sucrose, glucose, arabinose, mannose, glucosamine, maltose, sugar cane, alfalfa extracts, molasses, rum, and the like; salts of organic acids such as acetic acid, fumaric acid, adipic acid, propionic acid, citric acid, gluconic acid, malic acid, pyruvic acid, malonic acid and the like; alcohols such as ethanol, glycerol, and the like; oil or fat such as soybean oil, rice bran oil, olive oil, corn oil, and sesame oil. The amount of the carbon source added varies according to the kind of carbon source and is typically between 1 to 100 grams per liter of medium. The weight fraction of the carbon source in the composition may be about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition. Preferably, alfalfa is contained in the medium as a major carbon source, at a concentration of about 1 to 20% (w/v). More preferably, the alfalfa is at a concentration of about 5 to 12% (w/v).

Examples of suitable nitrogen sources include, without limitation, amino acids, yeast extract, alfalfa extract, tryptone, beef extract, peptone, potassium nitrate, ammonium nitrate, ammonium chloride, ammonium sulfate, ammonium phosphate, ammonia or combinations thereof. The amount of nitrogen source varies according to the nitrogen source, typically between 0.1 to 30 grams per liter of medium. The weight fraction of the nitrogen source in the composition may be about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

Examples of suitable inorganic salts include, without limitation, potassium dihydrogen phosphate, dipotassium hydrogen phosphate, disodium hydrogen phosphate, magnesium sulfate, magnesium chloride, ferric sulfate, ferrous sulfate, ferric chloride, ferrous chloride, manganous sulfate, manganous chloride, zinc sulfate, zinc chloride, cupric sulfate, calcium chloride, sodium chloride, calcium carbonate, sodium carbonate, and combinations thereof. The weight fraction of the inorganic salt in the composition may be about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

In some aspects, the compositions of the present disclosure may further comprise acetic acid or carboxylic acid. Suitable acetic acids include any known in the art including, without limitation, formic acid, acetic acid, propionic acid, butanoic acid, isobutyric acid, 3-methyl butanoic acid, methyl acetate ethyl acetate, propyl acetate, butyl acetate, isobutyl acetate, and 2-methyl butyl acetate. In some aspects, the acetic acid is included by using vinegar. The weight fraction of the acetic acid in the composition may be about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

In some aspects, the compositions disclosed herein can be frozen. The compositions of the present disclosure may be in liquid or dry form. In some aspects, the compositions disclosed herein can be a solid. In some aspects, the compositions disclosed herein can be a liquid. In some aspects, the composition may comprise an aqueous suspension of components. This aqueous suspension may be provided as a concentrated stock solution which is diluted prior to application or as a diluted solution ready-to-use. Also, the composition may be a powder, granules, dust, pellet or colloidal concentrate. Such dry forms may be formulated to dissolve immediately upon wetting or dissolve in a controlled-release, sustained-release, or other time-dependent manner. Also, the composition may be in a dry form that does not depend upon wetting or dissolving to be effective.

In some aspects, the composition of the present disclosure may comprise at least one optional excipient. Non-limiting examples of suitable excipients include antioxidants, additives, diluents, binders, fillers, buffering agents, mineral salts, pH modifying agents, disintegrants, dispersing agents, flavoring agents, nutritive agents, oncotic and osmotic agents, stabilizers, preservatives, palatability enhancers and coloring agents. The amount and types of excipients utilized to form the combination may be selected according to known principles of science.

In some aspects, the excipient may include at least one diluent. Non-limiting examples of suitable diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lacitol, mannitol, malitol, sorbitol, xylitol, maltodextrin, and trehalose.

In some aspects, the excipient may comprise a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, polypeptides, oligopeptides, and combinations thereof.

In some aspects, the excipient may include a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

In some aspects, the excipient may comprise a buffering agent. Representative examples of suitable buffering agents include, but are not limited to, MOPS, HEPES, TAPS, Bicine, Tricine, TES, PIPES, MES, Tris buffers or buffered saline salts (e.g., Tris buffered saline or phosphate buffered saline).

In some aspects, the excipient may include a disintegrant. Suitable disintegrants include, but are not limited to, starches such as cornstarch, potato starch, pregelatinized and modified starches thereof, sweeteners, clays, such as bentonite, microcrystalline cellulose, alginates, sodium starch glycolate, gums such as agar, guar, locust bean, karaya, pecitin, and tragacanth.

In some aspects, the excipient may include a dispersion enhancer. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

In some aspects, the excipient may include a lubricant. Non-limiting examples of suitable lubricants include minerals such as talc or silica; and fats such as vegetable stearin, magnesium stearate or stearic acid.

The weight fraction of the excipient(s) in the combination may be about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the combination.

In some aspects, the compositions of the present disclosure are stable at room temperature.

In some aspects, the consortia may be kept at a reduced temperature for storage and transportation without significantly compromising the viability of the live microorganisms. The consortia or compositions comprising the same may be refrigerated, frozen, or lyophilized. The compositions may be refrigerated at between 32° F. to 44° F.

In some aspects, the consortium or compositions comprising the same can be stored and transported in a frozen state. The live beneficial microorganisms can be reinvigorated quickly once the compositions are thawed and brought to ambient temperature, preferably with aeration and/or agitation.

In some aspects, the consortia can be lyophilized. The consortia is first frozen. Water is then removed amendments under vacuum. This process further reduces the weight of the composition for storage and transportation. The consortia of compositions comprising the same can be reconstituted and reinvigorated prior to application or administration.

In some aspects, the concentrated consortia, or compositions comprising the same can be diluted with water before application or administration. Diluted compositions can be stored for a prolonged period of time, e.g., as long as 30 days, without losing viability. To maintain the live beneficial microorganism in a substantially aerobic state, dissolved oxygen in the diluted compositions of the present disclosure are preferably kept at an optimal level. It is preferable to supply optimal amounts of oxygen to the diluted composition though slow aeration.

In some aspects, any of the composition disclosed herein can be administered in a form selected from the group consisting of powder, granules, a ready-to-use beverage, food bar, an extruded form, capsules, gel caps, and dispersible tablets.

Deposit information. A deposit of bacterium XXX, which is disclosed herein, will be made with the American Type Culture Collection (ATCC), 10801 University Blvd., Manassas, Va. 20110-2209. The date of deposit is and the accession number for the deposited bacterium XXX is ATCC Accession No. . All restrictions upon the deposit have been removed, and the deposit is intended to meet all of the requirements of 37 C.F.R. § 1.801-1.809. The deposit will be maintained in the depository for a period of 30 years, or 5 years after the last request, or for the effective life of the patent, whichever is longer, and will be replaced if necessary during that period.

Methods

Disclosed herein are methods of altering relative abundance of microbiota in a subject. In some aspects, the methods can comprise administering to the subject an effective dose of any of the composition disclosed herein, thereby altering the relative abundance of microbiota in the subject. In some aspects, the methods can comprise administering to the subject an effective dose of a composition comprising two or more strains of bacterium having a 16S rDNA sequence comprising SEQ ID NOs: 1, 2, 3, and 4, thereby altering the relative abundance of microbiota in the subject. In some aspects, the relative abundance of Clostridia bacteria can be increased. In some aspects, the relative abundance of Clostridia bacteria can be replaced. In some aspects, the compositions disclosed herein can be for replacing microbiota of a subject with a disease or disorder associated with an imbalanced microbiota (or dysfunctional microbiota or a microbiota that is decreased in functional diversity).

The method of altering microbiota can also include measuring the relative abundance of one or more microbiota in a sample from a subject. As used herein, the term “relative abundance” refers to the commonality or rarity of an organism relative to other organisms in a defined location or community. For example, the relative abundance can be determined by generally measuring the presence of a particular organism compared to the total presence of organisms in a sample.

The relative abundance of microbiota can be measured directly or indirectly.

Direct measurements can include culture based methods. Indirect measurements can include comparing the prevalence of a molecular indicator of identity, such as ribosomal RNA (rRNA) gene sequences, specific for an organism or group of organisms in relation to the overall sample. For example, a ratio of rRNA specific for Desulfovibrio and Clostridia in a total number of rRNA gene sequences obtained from a cecal sample can be used to determine the relative abundance of Desulfovibrio and Clostridia in the cecal sample.

As used herein, the term “microbiota” is used to refer to one or more bacterial communities that can be found or can exist (colonize) within a gastrointestinal tract of an organism. When referring to more than one microbiota, the microbiota may be of the same type (strain) or it may be a mixture of taxa. In some aspects, the methods and compositions disclosed herein that alter the relative abundance of microbiota from genera such as Clostridium in a gastrointestinal tract of a subject. The relative abundance microbiota can be altered by administering a pharmaceutical composition that includes microbiota from genera such as Clostridium or a compound that substantially increases the relative abundance of microbiota from genera such as Clostridium, or substantially decreases the relative abundance of microbiota from or orders such Desulfovibrionales.

In some aspects, the relative abundance of Clostridia can be increased in the subject by at least about 5%. In some aspects, the relative abundance of Clostridia can be increased in the subject by at least about 10%. In some aspects, the relative abundance of Clostridia can be increased in the subject by at least about 1%, 2%, 3%, 4%, 5%, 6%, 7%, 8%, 9%, or 10%. In some aspects, the relative abundance of at least one of species of Clostridia can be increased by 5%.

In some aspects, the methods disclosed herein can further comprise administering a second therapeutic agent to the subject. In some aspects, the second therapeutic agent can be one or more bacteriophages. In some aspects, the one or more bacteriophages can specifically target and kill Desulfovibrio. In some aspects, the second therapeutic agent can be one or more commercially available therapeutic agents that can be administered to treat obesity, Type II diabetes, and/or inflammatory bowel disease. In some aspects, the second therapeutic agent can be an anti-inflammatory agent.

Disclosed herein are methods of treating a subject with obesity. In some aspects, the methods can comprise administering to the subject any of the compositions disclosed herein, wherein the relative abundance of Clostridia is increased in the subject compared to the relative abundance prior to administration. In some aspects, the methods comprise administering to the subject a composition comprising two or more strains of bacterium having a 16S rDNA sequence comprising SEQ ID NOs: 1, 2, 3, and 4, wherein the relative abundance of Clostridia is increased in the subject compared to the relative abundance prior to administration.

Disclosed herein are methods of treating a subject with metabolic syndrome. In some aspects, the methods can comprise administering to the subject any of the compositions disclosed herein, wherein the relative abundance of Clostridia is increased in the subject compared to the relative abundance prior to administration. In some aspects, the methods can comprise administering to the subject a composition comprising two or more strains of bacterium having a 16S rDNA sequence comprising SEQ ID NOs: 1, 2, 3, and 4, wherein the relative abundance of Clostridia is increased in the subject compared to the relative abundance prior to administration.

Disclosed herein are methods of treating a subject with irritable bowel disease. In some aspects, the methods can comprise administering to the subject any of the compositions disclosed herein, wherein the relative abundance of Clostridia is increased in the subject compared to the relative abundance prior to administration. In some aspects, the methods can comprise administering to the subject a composition comprising two or more strains of bacterium having a 16S rDNA sequence comprising SEQ ID NOs: 1, 2, 3, and 4, wherein the relative abundance of Clostridia is increased in the subject compared to the relative abundance prior to administration.

Disclosed herein are methods of reducing weight gain in a subject. In some aspects, the methods can comprise administering to the subject any of the compositions disclosed herein, wherein the relative abundance of Clostridia is increased in the subject compared to the relative abundance prior to administration. In some aspects, the methods can comprise administering to the subject a composition comprising two or more strains of bacterium having a 16S rDNA sequence comprising SEQ ID NOs: 1, 2, 3, and 4, wherein the relative abundance of Clostridia is increased in the subject compared to the relative abundance prior to administration.

Disclosed herein are methods of inhibiting lipid absorption in a subject's small intestine. In some aspects, the methods can comprise administering to the subject any of the compositions disclosed herein, wherein the relative abundance of Clostridia is increased in the subject compared to the relative abundance prior to administration. In some aspects, the methods can comprise administering to the subject a composition comprising two or more strains of bacterium having a 16S rDNA sequence comprising SEQ ID NOs: 1, 2, 3, and 4, wherein the relative abundance of Clostridia is increased in the subject compared to the relative abundance prior to administration. In some aspects, any of the compositions disclosed herein can suppress one or more lipid absorption and/or synthesis genes. In some aspects, the lipid absorption genes can be CD36, FasN, Dgat, Srepbf1, SLC27a1, and SLC27a4.

Disclosed herein are methods of downregulating CD36 in a subject's liver. In some aspects, the methods can comprise administering to the subject any of the compositions disclosed herein, wherein the relative abundance of Clostridia is increased in the subject compared to the relative abundance prior to administration. In some aspects, the methods can comprise administering to the subject a composition comprising two or more strains of bacterium having a 16S rDNA sequence comprising SEQ ID NOs: 1, 2, 3, and 4, wherein the relative abundance of Clostridia is increased in the subject compared to the relative abundance prior to administration.

Disclosed herein are methods of reducing adiposity in a subject. In some aspects, the methods can comprise administering to the subject any of the compositions disclosed herein, wherein the relative abundance of Clostridia is increased in the subject compared to the relative abundance prior to administration. In some aspects, the methods can comprise administering to the subject a composition comprising two or more strains of bacterium having a 16S rDNA sequence comprising SEQ ID NOs: 1, 2, 3, and 4, wherein the relative abundance of Clostridia is increased in the subject compared to the relative abundance prior to administration.

Disclosed herein are methods of reducing weight gain in a subject. In some aspects, the methods can comprise administering to the subject any of the compositions disclosed herein, wherein the relative abundance of Clostridia is increased in the subject compared to the relative abundance prior to administration. In some aspects, the methods can comprise administering to the subject a composition comprising two or more strains of bacterium having a 16S rDNA sequence comprising SEQ ID NOs: 1, 2, 3, and 4, wherein the relative abundance of Clostridia is increased in the subject compared to the relative abundance prior to administration.

Disclosed herein are methods of reducing fat accumulation in a subject. In some aspects, the methods can comprise administering to the subject any of the compositions disclosed herein, wherein the relative abundance of Clostridia is increased in the subject compared to the relative abundance prior to administration. In some aspects, the methods can comprise administering to the subject a composition comprising two or more strains of bacterium having a 16S rDNA sequence comprising SEQ ID NOs: 1, 2, 3, and 4, wherein the relative abundance of Clostridia is increased in the subject compared to the relative abundance prior to administration.

Disclosed herein are methods of lowering body fat percentage and/or reducing visceral adipose tissue (VAT) mass in a subject. In some aspects, the methods can comprise administering to the subject any of the compositions disclosed herein, wherein the relative abundance of Clostridia is increased in the subject compared to the relative abundance prior to administration. In some aspects, the methods can comprise administering to the subject a composition comprising two or more strains of bacterium having a 16S rDNA sequence comprising SEQ ID NOs: 1, 2, 3, and 4, wherein the relative abundance of Clostridia is increased in the subject compared to the relative abundance prior to administration.

Disclosed herein are methods of decreasing blood glucose levels and/or reducing insulin resistance in a subject. In some aspects, the methods can comprise administering to the subject any of the compositions disclosed herein, wherein the relative abundance of Clostridia is increased in the subject compared to the relative abundance prior to administration. In some aspects, the methods can comprise administering to the subject a composition comprising two or more strains of bacterium having a 16S rDNA sequence comprising SEQ ID NOs: 1, 2, 3, and 4, wherein the relative abundance of Clostridia is increased in the subject compared to the relative abundance prior to administration.

In some aspects, in any of the methods disclosed herein, the subject has been identified as being in need of the treatment. In some aspects, the subject has obesity, metabolic syndrome, insulin deficiency, insulin-resistance related disorders, glucose intolerance, diabetes, or an inflammatory bowel disease. In some aspects, the inflammatory bowel disease can be Crohn's disease or ulcerative colitis. In some aspects, insulin-resistance related disorder can be diabetes, hypertension, dyslipidemia, or cardiovascular disease. In some aspects, diabetes can be Type I diabetes. In some aspects, diabetes can be Type II diabetes.

As used herein, the term “metabolic disorder” or “metabolic syndrome” refers to disorders, diseases, and conditions that are caused or characterized by abnormal weight gain, energy use or consumption, altered responses to ingested or endogenous nutrients, energy sources, hormones or other signaling molecules within the body or altered metabolism of carbohydrates, lipids, proteins, nucleic acids or a combination thereof. A metabolic disorder is associated with either a deficiency or excess in a metabolic pathway resulting in an imbalance in metabolism of nucleic acids, proteins, lipids, and/or carbohydrates. Factors affecting metabolism include, and are not limited to, the endocrine (hormonal) control system (e.g., the insulin pathway, the enteroendocrine hormones including GLP-1, PYY or the like), the neural control system (e.g., GLP-1 or other neurotransmitters or regulatory proteins in the brain) or the like. Some non-limiting examples can be obesity, diabetes, including type II diabetes, insulin-deficiency, insulin-resistance, insulin-resistance related disorders, glucose intolerance, syndrome X, inflammatory and immune disorders, osteoarthritis, dyslipidemia, metabolic syndrome, non-alcoholic fatty liver, abnormal lipid metabolism, cancer, neurodegenerative disorders, sleep apnea, hypertension, high cholesterol, atherogenic dyslipidemia, hyperlipidemic conditions such as atherosclerosis, hypercholesterolemia, and other coronary artery diseases in mammals, and other disorders of metabolism.

Disorders also included are conditions that occur or cluster together, and increase the risk for heart disease, stroke, diabetes, and obesity. Having just one of these conditions such as increased blood pressure, elevated insulin levels, excess body fat around the waist or abnormal cholesterol levels can increase the risk of the above mentioned diseases. In combination, the risk for coronary heart disease, stroke, insulin-resistance syndrome, and diabetes is even greater.

In some aspects, the step of administering any of the compositions disclosed herein can comprise delivering the composition to at least a stomach, a small intestine, or a large intestine of the subject. In some aspects, the composition can be administered orally.

In some aspects, the subject can be a human.

In some aspects, the cells of the consortia are active.

Kits

In some aspects, a kit is disclosed comprising one or more bacteria, strain or microorganism capable of for reducing adiposity in a subject, reducing weight gain and/or fat accumulation in a subject, lowering body fat percentage and/or reducing visceral adipose tissue (VAT) mass in a subject, decreasing blood glucose levels and/or reducing insulin resistance in a subject, inhibiting lipid absorption in a subject's small intestine, downregulating CD36 in a subject's liver and suppressing expression of lipid absorption genes within intestinal epithelia in a subject

### EXAMPLES

### Example 1: T Cell-Mediated Regulation of the Microbiota Protects Against Obesity

Abstract: The microbiota influences host metabolism and obesity, yet organisms that protect from disease remain unknown. During studies interrogating an immune pathway that regulates microbiota composition, the development of age-associated metabolic-syndrome driven by the microbiota was observed. Expansion of Desulfovibrio and loss of Clostridia were important features associated with obesity in this model and replacement of Clostridia rescues obesity. T-cell dependent events were required to prevent loss of Clostridia and expansion of Desulfovibrio. Inappropriate IgA targeting of Clostridia and increased Desulfovibrio antagonized the colonization of beneficial Clostridia. Transcriptional and metabolic analysis revealed enhanced lipid absorption in the obese host. Colonization of germfree animals with Clostridia, but not Desulfovibrio, downregulated the expression of genes controlling lipid absorption and reduced adiposity. Moreover, supernatants from Clostridia suppressed the expression of lipid absorption genes within intestinal epithelia. Reduced Clostridia and increased Desulfovibrio were microbiota features found in humans with metabolic syndrome and obesity. Thus, immune control of the microbiota appears to maintain beneficial microbial populations that function to constrain lipid metabolism to prevent metabolic defects.

Introduction. Over the past century, obesity and metabolic syndrome have developed into a global epidemic. Currently, over 1.9 billion people are obese and at risk of developing metabolic dysfunctions such as type II diabetes, cardiovascular, and liver disease (D. Mozaffarian et al., Circulation 131, e29-322 (2015)).Multiple studies have highlighted a role for immune-system regulation of metabolic disease. These reports have largely focused on the role of inflammatory responses during obesity. They reported increased macrophage infiltration and a reduction in regulatory T cells within the adipose tissue during weight gain (M. F. Gregor, and G. S. Hotamisligil, Annu Rev Immunol 29, 415-445 (2011); and F. Emanuela et al., Journal of nutrition and metabolism 2012, 476380 (2012)). However, a number of human studies suggest that suboptimal immune responses are also associated with metabolic syndrome and obesity. Indeed, obese adults show deficient immune responses to immunizations, increased incidence of infection and reduced mucosal IgA levels, suggesting that effective immunity cannot be mounted within these individuals (A. Pallaro et al., J Nutr Biochem 13, 539 (2002); A. Must et al., JAMA 282, 1523-1529 (1999); D. C. Nieman et al., J Am Diet Assoc 99, 294-299 (1999); D. N. McMurray, P. A. Beskitt, S. R. Newmark, Int J Obes 6, 61-68 (1982); J. Hirokawa et al., Biochem Biophys Res Commun, 235, 94-98 (1997); and S. Tanaka et al., Int J Obes Relat Metab Disord 17, 631-636 (1993)). The mechanisms by which defective immune reactions influence metabolic disease remain unclear.

The microbiota has emerged as an important regulator of metabolism within the mammalian host, and the composition of the microbiota in obese individuals is sufficient to confer metabolic defects when transferred into animals (P. J. Turnbaugh et al., Nature 444, 1027-1031 (2006)). In particular, reductions in the gene richness of the microbiota have been reported during metabolic disease, including decreased butyrate and methane production. Conversely, some microbiota functions, such as hydrogen sulfide and mucus degradation, are enhanced in individuals with metabolic disease (J. Qin et al., Nature 490, 55-60 (2012)). It has been recently shown that gut immune responses are important in regulating the composition of the microbiota (J. L. Kubinak et al., Cell Host Microbe 17, 153-163 (2015); and S. Kawamoto et al., Immunity 41, 152-165 (2014)). IgA, in particular, functions to constrain the outgrowth of certain microbes and diversify the microbiota; changes in IgA binding of microbes or, even slight reductions in gut IgA, can negatively affect diversity (J. L. Kubinak et al., Cell Host Microbe 17, 153-163 (2015); S. Kawamoto et al., Immunity 41, 152-165 (2014); and S. Wang et al., Immunity 43, 289-303 (2015)). Thus, defective immune control of the microbiota may contribute to metabolic disease.

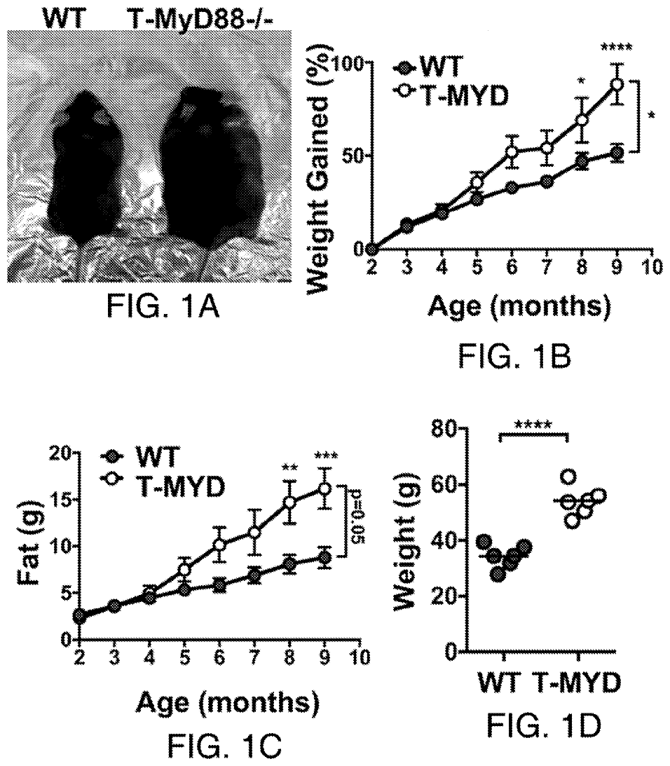

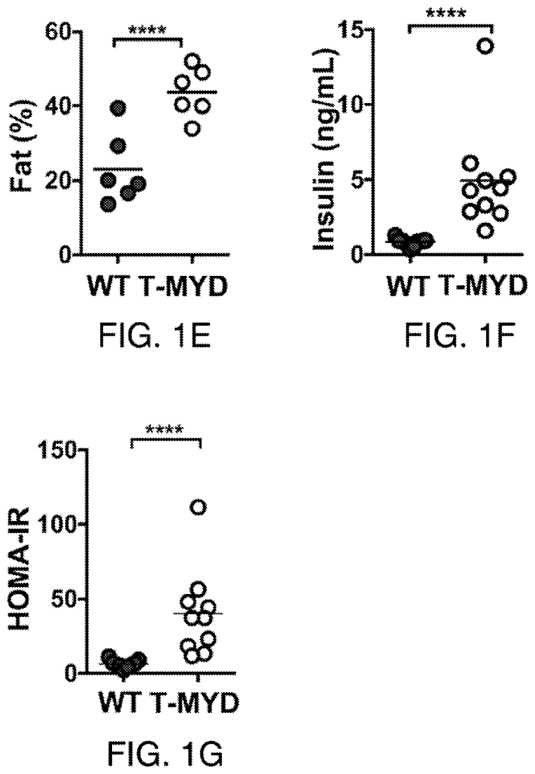

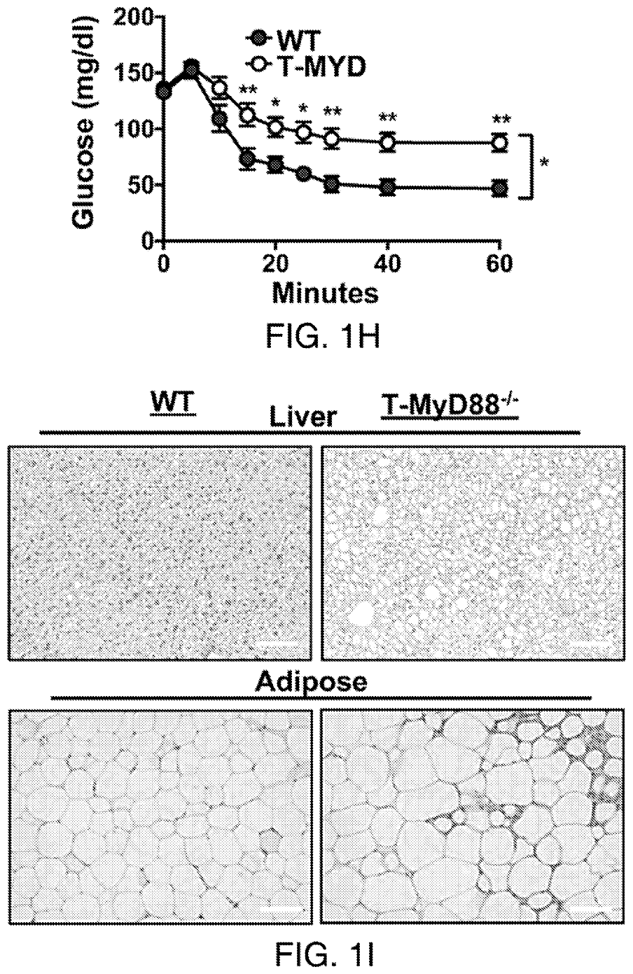

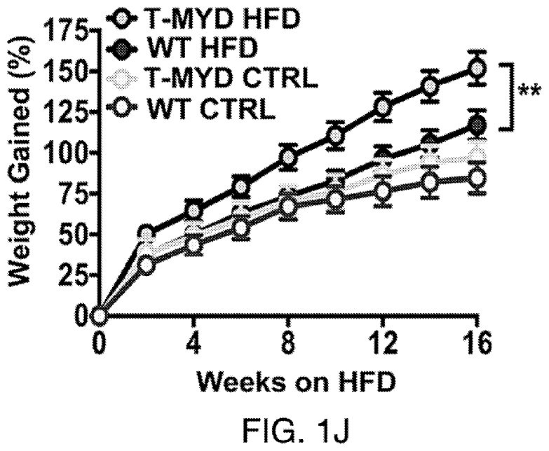

Results. Recently, a molecular pathway that instructs the appropriate development of T cell-dependent IgA targeting of the microbiota was identified. Animals that possess a T cell specific ablation of the innate adaptor molecule, Myd88 (T-Myd88^{-/-} mice) have defective T follicular helper (TFH) cell development and IgA production within the gut. This was associated with dysregulated IgA targeting of gut microbes and compositional differences within the microbiota between genotypes (J. L. Kubinak et al., Cell Host Microbe 17, 153-163 (2015); and S. Wang et al., Immunity 43, 289-303 (2015)). During these studies, it was observed that older T-Myd88^{-/-} mice were consistently obese compared to their wild-type controls (FIG. 1A). Despite being fed a standard chow diet, T-Myd88^{-/-} mice exhibited significantly increased weight gain and fat accumulation as they aged (FIGS. 1B and C and FIGS. 7A and B). By one year of age, male T-Myd88^{-/-} mice weighed up to 60 g and exhibited a 50% body fat composition based on NMR analysis (FIGS. 1D and E).

T-Myd88^{-/-} animals developed many of the metabolic disease co-morbidities found in humans (F. X. Pi-Sunyer, Med Sci Sports Exerc 31, S602-608 (1999)). Although one-year-old T-Myd88^{-/-} mice raised on a standard diet cleared glucose to similar levels as their WT counterparts (FIG. 7C), they had higher levels of circulating insulin, resulting in a higher HOMA-IR index (FIGS. 1F and G). Moreover, when challenged with additional insulin, T-Myd88^{-/-} mice failed to clear glucose with similar kinetics as WT animals, indicating the development of insulin resistance (FIG. 1H). Food intake was decreased in T-Myd88^{-/-} mice at two months of age compared to WT controls but was equivalent in one-year old animals (FIGS. 7D and E). Additionally, although energy expenditure was decreased in young mice, these changes did not persist over time (FIG. 7D). Movement was also similar between WT and T-Myd88^{-/-} mice at both ages and a modest increase in heat production was measured in older T-MyD88^{-/-} mice compared to WT controls suggesting that these are not the primary cause of increased weight gain as seen in other models (FIGS. 7F and G) (M. Vijay-Kumar et al., Science 328, 228-231 (2010)). T-Myd88^{-/-} mice also developed fatty liver disease and displayed inflammatory phenotypes within the adipose tissue that were marked by crown-like structures and dysregulated adipocyte size (FIG. 1I). Obesity on a standard mouse chow diet requires months to develop. In contrast, when animals were placed on a high-fat diet (HFD, 45% fat), T-Myd88^{-/-} animals accumulated more weight and visceral adipose tissue (VAT) mass than WT mice as early as 8 weeks after initiation of the diet (FIG. 1J and FIGS. 8A and B). Thus, T-Myd88^{-/-} animals are prone to developing metabolic syndrome and obesity, which can be accelerated by the increased intake of dietary fat.

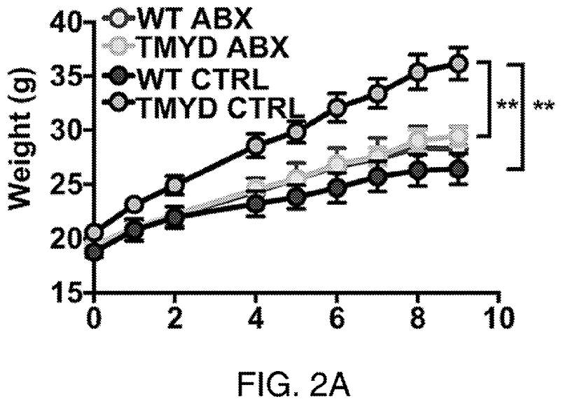

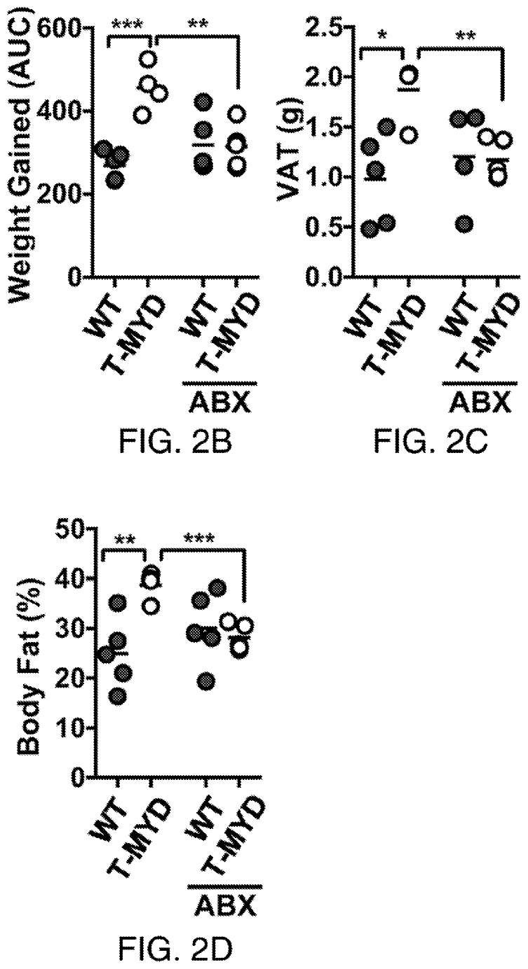

The composition of the T-Myd88^{-/-} microbiota is distinct from WT in young animals (J. L. Kubinak et al., Cell Host Microbe 17, 153-163 (2015)). The microbiota is a known contributor to metabolic function and has been linked with the development of human obesity (S. Ussar et al., Cell Metab 22, 516-530 (2015); and E. Le Chatelier et la., Nature 500, 541-546 (2013)). To initially determine if the microbiota was involved in the metabolic syndrome seen in T-Myd88^{-/-} mice, WT and T-Myd88− mice were placed on broad-spectrum antibiotics while feeding them a HFD. WT mice exhibited no difference in weight gain on antibiotics. In contrast, weight gain was completely rescued by antibiotic treatment in T-Myd88^{-/-} animals (FIGS. 2A and B). This was accompanied by a reduction in their body fat percentage and VAT mass to levels similar to the fat accumulation observed in lean animals (FIGS. 2C and D).

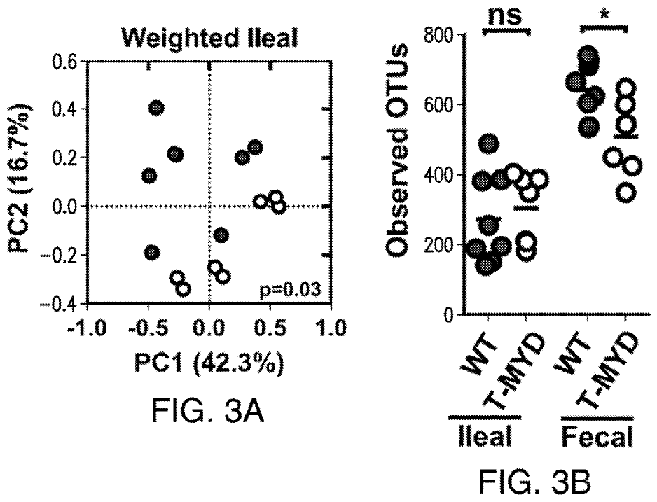

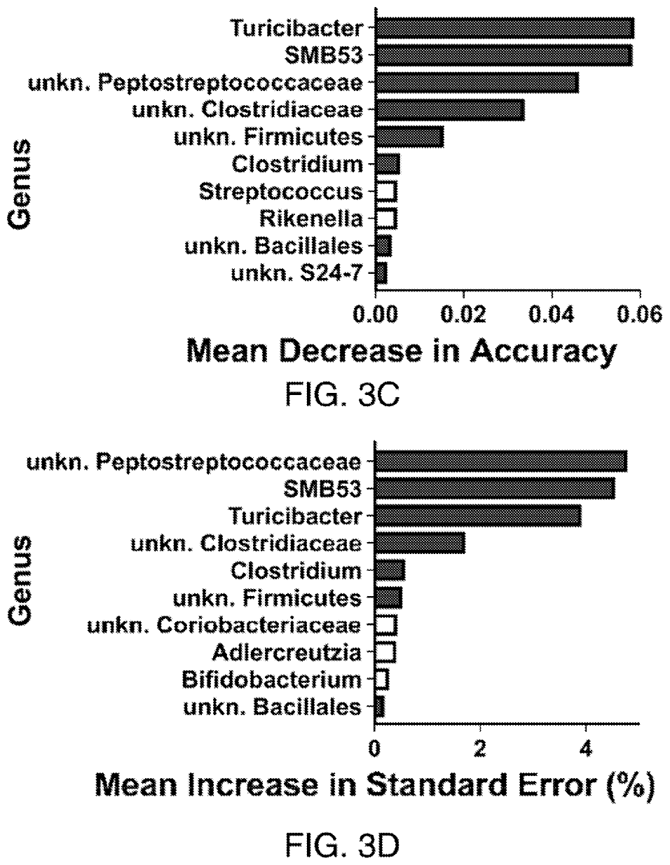

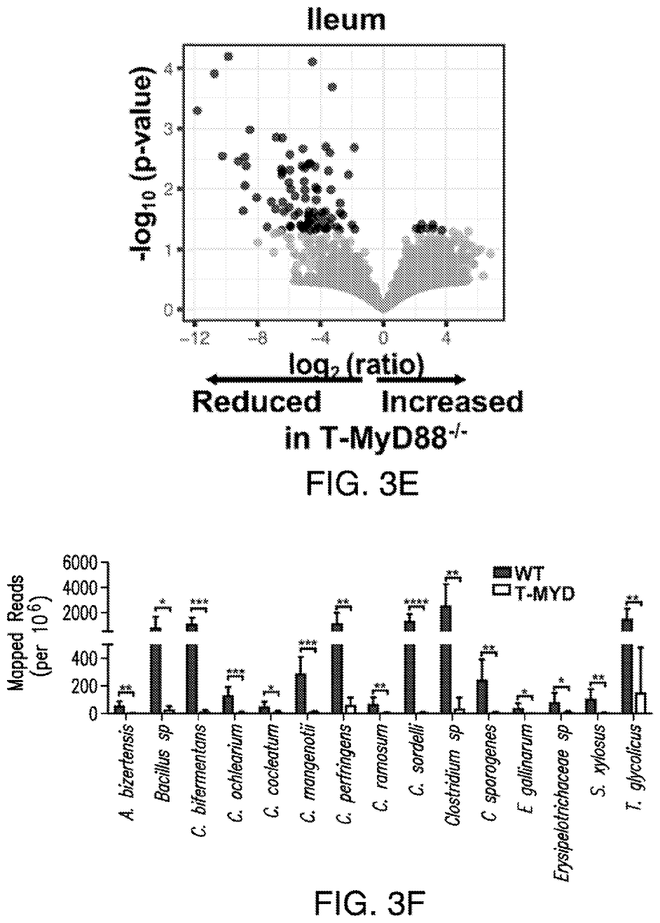

In order to determine the features of the microbiota that affect metabolic syndrome in T-Myd88^{-/-} mice, 16S rRNA gene sequencing was performed on normal-chow-fed, aged animals to assess the taxonomic composition and diversity of the microbiota in obese T-Myd88^{-/-} mice. There were significantly different communities in the ileum and fecal contents of aged WT and T-Myd88^{-/-} mice (FIG. 3A and FIG. 9A). Additionally, there was a slightly reduced species richness in the feces of aged mice (FIG. 3B). In order to identify organisms that could explain the major differences between WT and T-Myd88^{-/-} microbiota communities, a random forest analysis was performed on the 16S rRNA data. The fecal microbiota was able to accurately classify genotype with 86% accuracy, whereas the ileal microbiota predicted genotype with 100% accuracy. Members of the microbiota that had the strongest influence on accuracy mostly belonged to the broad taxonomic class Clostridia and were enriched in WT mice compared to T-Myd88^{-/-} mice (FIG. 3C and FIG. 9B). An additional random forest approach indicated that fecal and ileal microbiota could predict total weight with R²=0.5 and R²=0.76, respectively, with many members of Clostridia strongly influencing this prediction (FIG. 3D and FIG. 9B). Compared to WT mice, T-Myd88^{-/-} mice showed broad reductions in diversity and overall abundance of multiple Clostridia taxa, including Dorea, SMB53, unclassified Peptostreptococcaceae, and Clostridium (FIG. 9C).

Compositional shifts in the microbiota, including reduced microbial diversity, can have negative effects on the functionality of the microbiota and have been correlated with a number of western lifestyle-associated diseases including metabolic syndrome (M. Vij ay-Kumar et al., Science 328, 228-231 (2010); and S. Ussar et al., Cell Metab 22, 516-530 (2015)). Additionally, individuals harboring a microbiota with lower gene richness are more likely to be obese (E. Le Chatelier et al., Nature 500, 541-546 (2013)). In fecal and ileal microbial transcriptomes, the representation of transcripts from a number of gene families within T-Myd88^{-/-} animals was generally reduced. As there were the same number of organisms detected within the ileum by 16S rRNA gene sequencing, this supports the hypothesis that the microbiota at these sites has reduced metabolic functionality (FIG. 3E and FIG. 10A). A comparable proportion of total reads uniquely mapped to reference genomes between the two genotypes, suggesting the same coverage of transcriptomes in the animals. However, the proportion of reads mapped to the Clostridiaceae reference genomes in ileal and fecal transcriptomes of T-Myd88^{-/-} mice was, in particular, strikingly reduced (FIG. 3F and FIGS. 10B and C). Thus, Clostridia present in the obese animals have a reduced functional contribution to the microbiome. Furthermore, obesity is associated with a loss of microbial functional diversity within the Clostridia as has similarly been reported in humans with metabolic disease (J. Qin et al., Nature 490, 55-60 (2012)).

As loss of important Clostridia organisms may play a role during disease, a co-housing experiment was performed to determine whether microbial transfer could rescue obesity (FIG. 11A). As mice are coprophagic, co-housing allows for efficient and frequent transfer of microbes between genotypes and has known homogenizing effects on the microbiota. WT or T-Myd88^{-/-} animals were either housed together with animals of the same genotype or co-housed with animals of the opposite genotype upon weaning. Prior to co-housing, T-Myd88^{-/-} mice had a distinct microbiota composition, and one week of cohousing caused mixing of the two communities (FIG. 12A).

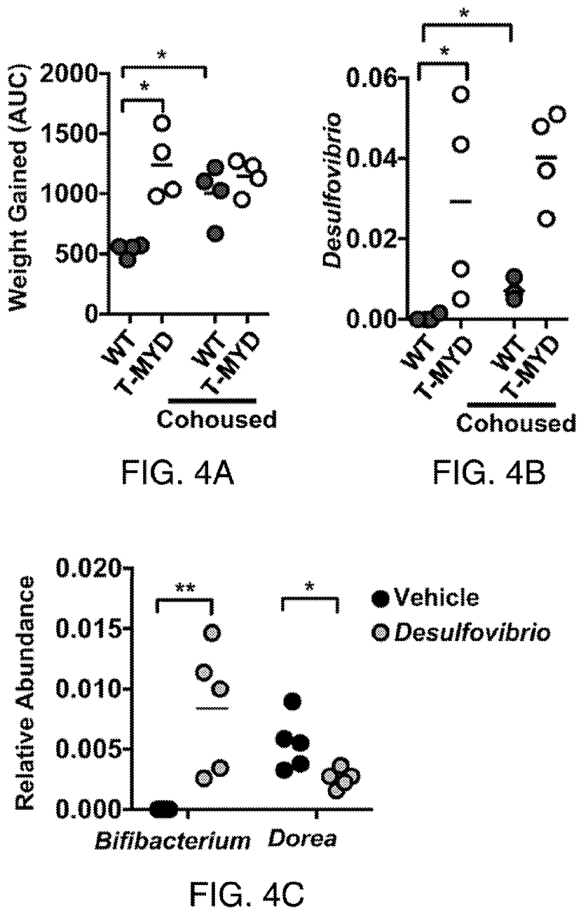

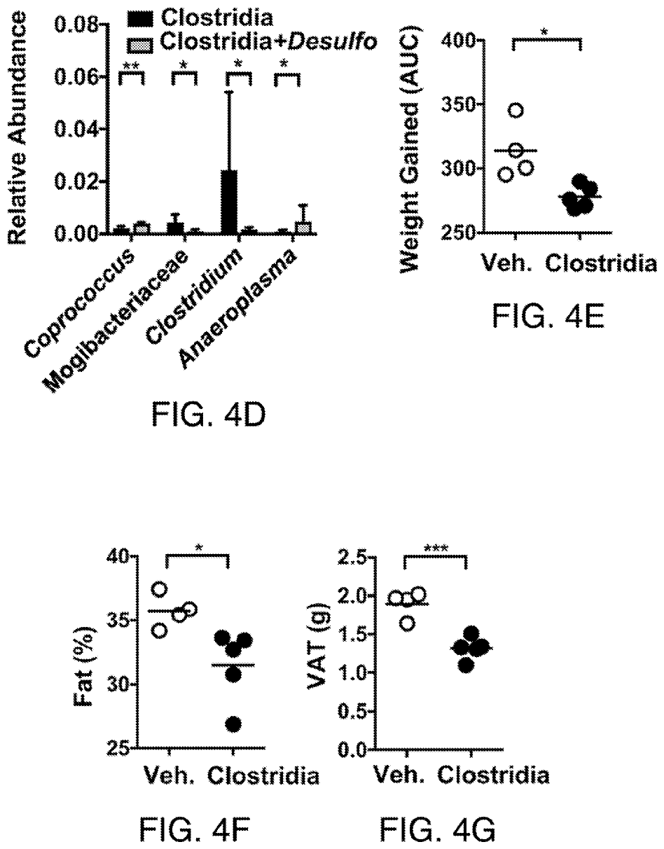

After 1 week, animals were placed on a HFD and monitored for signs of fat accumulation. Compared to separated WT mice, T-Myd88^{-/-} mice and any animal cohoused with them gained significantly more weight, developed insulin resistance, and had increased VAT and total body fat (FIG. 4A and FIGS. 11B to E). Furthermore, after three months, the microbiota from cohoused WT animals became significantly distinct from separately housed WT mice and showed greater similarity to the microbiota of separately housed T-Myd88^{-/-} (FIG. 12B). Thus, a transferable component of the microbiota formed in a T-Myd88^{-/-} animal that can cause metabolic syndrome in an otherwise healthy WT animal.

As differences in weight gain of cohoused animals were detected within the first 3 weeks, the differences in microbial composition that were detectable at both the early and final time points were a focus. After three months of cohousing, Desulfovibrio, Lactobacillales, and Bifidobacterium pseudolongum were present at greater abundances within cohoused WT mice (FIG. 4B and FIGS. 12C and D). However, the Desulfovibrio genus showed significantly greater abundance in separately housed T-Myd88^{-/-} animals and co-housed animals after just one week of co-housing (FIG. 12E). Desulfovibrio are mucolytic d-proteobacteria that produce hydrogen sulfide as a byproduct of disulfide-bond degradation within mucin (G. R. Gibson, G. T. Macfarlane, J. H. Cummings, J Appl Bacteriol 65, 103-111 (1988); M. C. Pitcher, J. H. Cummings, Gut 39, 1-4 (1996); F. E. Rey et al., Proc Natl Acad Sci USA 110, 13582-13587 (2013); and M. S. Desai et al., Cell 167, 1339-1353 (2016)). In addition to its association with inflammatory bowel disease (IBD), increased colonization of Desulfovibrio and genes associated with hydrogen sulfide production are detected in patients with type II diabetes and obesity (J. Qin et al., Nature 490, 55-60 (2012)). Thus, the community changes in obese mice mimics much of what is seen in humans and suggests that loss of Clostridia and increases in Desulfovibrio is highly relevant to metabolic disease (J. Qin et al., Nature 490, 55-60 (2012); J. Zierer et al., Nat Genet 50, 790-795 (2018); and S. M. Harakeh et al., Front Cell Infect Microbiol 6, 95 (2016)).

Cohousing of WT mice with T-Myd88^{-/-} animals that leads to obesity is also associated with reduced colonization of members of Clostridia in WT animals (FIG. 12F). Therefore, it was tested whether Desulfovibrio colonization may reduce the abundance of these organisms.

Specific-pathogen-free (SPF) mice were colonized for one week with Desulfovibrio desulfuricans subsp. desulfuricans, a strain that has a 16S rRNA gene sequence similarity of greater than 97% to the Desulfovibrio identified in the mice. The results show that WT SPF animals had significant reductions in the Clostridiales family Lachnospiraceae and genus Dorea (FIG. 4C and FIG. 13A). Colonization with Desulfovibrio did not result in an overall reduction to all organisms as there was a significant increase in Bifidobacterium (FIG. 4C). As these changes to the community could be an indirect effect of Desulfovibrio colonization, it was tested whether Desulfovibrio could influence the colonization of Clostridia members in a germfree system.

Germfree animals colonized with chloroform-treated fecal slurries were enriched for Clostridiaceae and Lachnospiraceae (FIG. 14). This community was then analyzed in the presence or absence of D. desulfuricans. Desulfovibrio colonization lead to a significant reduction in Clostridium, a genus which strongly influenced the predictive accuracy of both genotype and weight (FIG. 4D). Thus, an expansion of Desulfovibrio species, as seen in T-Myd88^{-/-} mice and humans with type II diabetes, can antagonize the colonization of microbes associated with leanness.

We sought to identify whether reintroducing these lean-associated microbes could protect against obesity within T-Myd88^{-/-} mice. Treatment of obesity-prone T-Myd88^{-/-} animals every other day with a cocktail of spore-forming bacteria significantly reduced weight gain and fat accumulation (FIGS. 4E and F). At the end of 3 months, T-Myd88^{-/-} mice treated with spore-forming microbes had a lower body fat percentage and a reduced VAT mass when compared to untreated T-Myd88^{-/-} mice (FIGS. 4F and G). Thus, loss of Clostridia is causally associated with obesity and metabolic syndrome in T-Myd88^{-/-} mice.

Microbiota formed during defective gut immunity appears to result in metabolic syndrome. Although co-housing of animals for 12 weeks led to the transmission of obesity into WT hosts, fecal transplants from T-Myd88^{-/-} into WT germfree recipients was insufficient to transfer obesity (FIG. 15A). Additionally, when either SPF WT or T-Myd88^{-/-} pregnant dams were co-housed with germfree WT pregnant dams, the resulting colonized pups separated at weaning did not transmit the obesity phenotype (FIG. 15A). Thus, it was tested whether immune defects in T-Myd88^{-/-} mice were necessary to allow the persistence of the obesogenic microbiota. In contrast, the microbiota was appropriately controlled in the presence of a fully intact immune system. Tcrb mice, which lack endogenous T cells, were depleted of endogenous microbiota with broad spectrum antibiotic treatment and then colonized with a 1:1 mixture of WT and T-Myd88^{-/-} microbiota prior to adoptive transfer of either WT or T-Myd88^{-/-} CD4⁺ T cells (FIG. 15B). Mice were separated into individually housed cages so that microbiota formation would not be influenced by the presence of other animals within the cage and each microbial community would be shaped independently. Despite the fact that these mice were initially colonized with the same microbiota, Tcrb⁻¹⁻ mice given T-Myd88^{-/-} CD4⁺ T cells gained significantly more weight when compared to Tcrb mice given WT CD4⁺ T cells (FIG. 5A). Thus, defects in Myd88 signaling within T cells drives the metabolic defects in animals. Ten percent of bacteria were coated by IgA within Tcrb^{-′-} mice, demonstrating the importance of T cells for IgA targeting of the microbiota (FIG. 3B). However, 1 week post-T cell transfer, mice given WT T cells showed a threefold increase in IgA-bound microbes (FIG. 15C). IgG1 or IgG3 responses against the microbiota took longer to develop but were detectable 8 weeks post-T cell transfer (FIG. 15D to F). Although, total IgA levels were similar in the animals within this experimental setting, IgA and IgG1 binding to the microbiota was defective in animals receiving knockout T cells (FIG. 5B and FIGS. 15E and G). Targeting of the microbiota by IgG3, which is believed to be governed by T-cell-independent mechanisms, was not defective in Tcrb mice receiving T-Myd88^{-/-} T cells (FIG. 15H) (M. A. Koch et al., Cell 165, 827-841 (2016)). The microbiota composition increasingly differed between genotypes over time (FIG. 5C). Moreover, community changes in animals receiving T cells from obesogenic mice are similar those observed in T-Myd88^{-/-} animals. Indeed, there was a significant negative correlation between the abundance of Desulfovibrionaceae and Clostridiaceae in both genotypes.

Animals receiving T-Myd88^{-/-} T cells were ultimately colonized with significantly fewer Clostridiaceae despite starting with the same microbiota admixture (FIGS. 5D and E). Three taxa at the genus level were differentially targeted by IgA including the Oscillospira genus of Clostridia, whereas most Clostridia genera were highly variable at this level of taxonomic resolution (FIG. 16A). The IgA-binding index was assessed at the finer OTU-level (97% similarity) and found an enrichment of Clostridia-classified OTUs differentially targeted by IgA in animals receiving T-Myd88^{-/-} T cells (FIG. 5F). Trending increases in IgA targeting of Desulfovibrio was observed (FIGS. 16A and B). Thus, reductions in Clostridia and their functional contributions may arise from a combination of inappropriate targeting by IgA and the expansion of Desulfovibrio.

To support the hypothesis that antibody responses influence metabolic defects, the obesogenic microbiota was transferred into either antibiotic-treated WT or Rag1^{-′-} animals. Indeed, the transfer of the obesogenic microbiota to WT mice did not confer the phenotype, whereas transfer into Rag1^{-′-} animals, which lack antibodies, resulted in significantly greater weight gain compared to animals receiving WT microbiota (FIG. 5G and FIG. 15I). TFH are T cells that function to instruct antibody class switching and mutation within B cells in germinal centers. It was previously established that the T cell developmental defect in T-Myd88^{-/-} mice was within TFH cells.

T-Myd88^{-/-} animals receiving Bcl6^{-′/-} T cells, which cannot differentiate into TFH cells, weighed significantly more compared to animals receiving WT T cells (FIG. 5H) (S. Crotty, Annu Rev Immunol 29, 621-663 (2011)). Thus, T cells that do not have the capacity to develop into TFH cells fail to rescue the obesity phenotype. Appropriate TFH cell function is therefore required to regulate the microbiota to prevent obesity.

Short-chain fatty acids (SCFAs) are a well-studied microbiota-dependent mechanism that influences host metabolism. However, SCFA production did not differ between WT and T-Myd88^{-/-} animals (FIG. 17A). Increased intestinal permeability and leakage of bacterial products that induce low-grade inflammation within adipose tissue has also been proposed (F. E. Rey et al., Proc Natl Acad Sci USA 110, 13582-13587 (2013); and P. D. Cani et al., Diabetes, 56, 1761-1772 (2007)). However, differences in bacterial ligands within the serum of T-Myd88^{-/-} animals were not detected. Furthermore, placement of T-Myd88^{-/-} on a diet infused with an anti-inflammatory, 5-ASA, (H. Luck et al., Cell Metab 21, 527-542 (2015)) failed to rescue weight gain (FIG. 17B). Liver RNA-seq and gene set enrichment analysis (GSEA) revealed that, despite animals being fed a standard mouse chow, pathways involved in lipid metabolism, including glycerolphospholipid and glycerolipid metabolism, were the most significantly enriched pathways within T-Myd88^{-/-} animals compared to WT controls (FIG. 6A). Particularly, expression of genes required for the synthesis of lipids, including Fasn, Dgat2, and Srebpf1, and genes involved in lipid absorption including Slc27a4 and Cd36, were highly upregulated within the liver of T-Myd88^{-/-} animals (FIG. 6B). Although CD36 was upregulated in T-Myd88^{-/-} animals, antibiotic treatment significantly downregulated CD36 expression (FIG. 6C). Moreover, Clostridia treatment of obese T-Myd88^{-/-} animals produced a significant downregulation of CD36, suggesting that Clostridia function to reduce lipid uptake (FIG. 6D). Indeed, gnotobiotic animals colonized with the Clostridia consortia that had significant reductions within hepatic CD36 expression when compared to germfree mice (FIG. 6E). Thus lipid uptake in T-Myd88^{-/-} appears to be in a microbiota-dependent manner.

Colonization of germfree animals with Clostridia significantly downregulates both CD36 and FASN within the small intestine (FIGS. 6F and G), suggesting that Clostridia influence lipid absorption and metabolism within the gut. Moreover, cell-free supernatant (CFS) collected from the cultured Clostridia consortia significantly downregulated CD36 in cultured intestinal epithelial cells (IECs) (FIG. 6H). In contrast, CFS collected from cultured Desulfovibrio species directly elevated the expression of CD36 on IECs (FIG. 6H). Furthermore, germfree animals mono-associated with the Clostridia consortia showed a significant decrease in body fat percentage compared to animals mono-associated with Desulfovibrio or germfree animals (FIG. 6I). Notably, the addition of Desulfovibrio to germfree mice colonized with the Clostridia consortia alone led to an increase in body fat percentage and CD36 expression in the small intestine (FIGS. 6J and K). Thus, the microbiota can directly regulate lipid metabolism within gut epithelia.

Supporting increased lipid absorption, HFD-fed T-Myd88^{-/-} had significant decreases in several long-chain fatty acids (LCFAs) within the cecum and concomitant increases in the serum (FIGS. 6L and M). Comparison of lumenal lipid profiles and 16S sequencing revealed opposing correlations between Desulfovibrio and members of Clostridia and the abundance of LCFAs and other lipids. The depletion of LCFAs within the cecal content was significantly correlated with the presence of Desulfovibrio. In contrast, multiple members of Clostridia, including SMB53 and Dorea, were associated with LCFA accumulation (FIG. 17C), further supporting the hypothesis that microbial composition can regulate lipid absorption. Thus, the loss of particular Clostridia species seen in individuals with obesity and T2D may lead to increased intestinal absorption and metabolism of fats, highlighting the importance of an appropriate microbiota composition to health.

Discussion. The microbiota has been implicated in a wide variety of autoimmune and metabolic conditions. However, these diseases are not always associated with the acquisition of a pathogenic organism, and instead the loss of beneficial species has been proposed to be a causative factor (I. Cho et al., Nature 488, 621-626 (2012)). Mechanisms leading to the loss of beneficial bacteria can include antibiotic use, increased sanitation and a low-fiber diet (N. M. Koropatkin, E. A. Cameron, E. C. Martens, Nat Rev Microbiol 10, 323-335 (2012)). The results described herein indicate that another mechanism to maintain healthy microbial communities is through appropriate immune control of these populations within the intestine. The microbiota formed within T-Myd88^{-/-} animals mirrors the dysbiosis seen in individuals with type II diabetes and obesity, including an expansion of Desulfovibrio and a loss of Clostridia (J. Qin et al., Nature 490, 55-60 (2012)). Although comprehensive human studies are lacking, individuals with obesity and type II diabetes have also been reported to have lower mucosal IgA and decreased responses to immunizations. This suggests that these individuals have a sub-optimal, but not completely deficient, immune response to gut microbiota that, coupled with dietary deficiencies, leads to metabolic disease. These data suggest that T cell-dependent targeting of the microbiota is important for the maintenance of a healthy community. Although IgA binding of bacteria is typically thought to lead to its eradication, IgA can regulate the functional gene expression of certain bacteria and even aid in mucosal association of certain commensals (G. P. Donaldson et al., Science 360, 795-800 (2018); T. C. Cullender et al., Cell Host Microbe 14, 571-581 (2013); and D. A. Peterson, N et al., Cell Host Microbe 2, 328-339 (2007))/ Indeed, the results show that despite lower levels of IgA in T-Myd88^{-/-} animals, Desulfovibrio and several Clostridia species display increased IgA coating. Thus, inappropriate targeting of Clostridia by IgA may either reduce their colonization or change their metabolic functions to influence development of obesity.

Additionally, several Clostridia are targeted less by IgA. Interestingly, a recent evaluation of the microbiota within individuals with IgA deficiency showed a significant reduction in colonization by several Clostridia (J. Fadlallah et al., Sci Transl Med 10, (2018)). Therefore, IgA may also function to enhance colonization of some Clostridia species as has been shown for Bacteroides fragilis (G. P. Donaldson et al., Science 360, 795-800 (2018)). The mechanism by which Desulfovibrio expands in this model and in individuals with metabolic syndrome is still unclear.

The results described herein, however, indicate that this expansion can directly influence the colonization of specific Clostridia members, although how this occurs remains enigmatic. Understanding how IgA targeting of gut microbes influences their colonization and function in a germfree setting may provide insight into how the immune system influences this microbial relationship. As members of Clostridia are increasingly recognized in several settings (24), it will be important to determine how colonization by other micro-organisms and the immune system together influence the function of Clostridia.

CD36 is an important regulator of lipid absorption within the intestine and its deficiency results in resistance to the development of obesity and metabolic syndrome upon HFD feeding (M. Buttet et al., PLoS One 11, e0145626 (2016); and M. Buttet et al., Biochimie 96, 37-47 (2014)). Increased expression of CD36 within the human liver is associated with fatty liver disease. Furthermore, individuals with polymorphisms in CD36, which produce just a twofold decrease in its expression within the gut, are resistant to metabolic disease (L. Love-Gregory, N. A. Abumrad, Curr Opin Clin Nutr Metab Care 14, 527-534 (2011)). Thus, relative expression levels of CD36 are important for lipid absorption and homeostasis within mammals. Recent studies have demonstrated that the microbiota can upregulate host absorption of lipids within the intestine through enhanced CD36 expression (Y. Wang et al., Science 357, 912-916 (2017)). However, it was found that bacteria may also be able to restrain host lipid absorption.

Thus, gut bacteria can differentially regulate lipid metabolism. Indeed, products secreted by Desulfovibrio upregulate CD36 expression, whereas products produced by Clostridia can downregulate CD36 expression. Therefore, the loss of organisms that function to temper CD36 expression may lead to the inappropriate absorption of lipids, which can accumulate over time, leading to obesity and metabolic syndrome. Further characterization of the interaction of organisms such as Desulfovibrio and Clostridia as well as identification of the molecules secreted that influence CD36 expression may inform future targeted therapies.

Materials and Methods. Mice. C57B1/6 Myd88^{LoxP/LoxP} mice (Jackson Laboratories) were crossed to C57B1/6 CD4-Cre animals (Taconic) to produce Myd88^{+/+}; CD4-Cre⁺ mice (WT) and Myd88LoxP/LoxP; CD4-Cre⁺ (T-MyD88^{-/-}) animals. Age-matched male mice were used to compare the spontaneous weight phenotype, including immune and microbiota responses, on a standard diet. Age-matched male and female mice were used to compare the weight phenotype, including immune and microbiota responses, on a high-fat diet (HFD). To measure T cell-dependent shaping of the microbiota, 4-week old Tcrb^{-/-} mice (Jackson Laboratories) were used. To investigate Desulfovibrio desulfuricans-dependent shaping of the microbiota, 6-week-old WT C57B1/6 mice (Jackson Laboratories) were used or age-matched CD4-Cre⁺ (WT) mice. To measure microbiota effects on weight gain in immunodeficient mice, 4-week old Rag1^{-/-} mice (Jackson Laboratories) were used. GF mice were maintained in sterile isolators and verified monthly for GF status by plating and PCR of feces. GF C57B1/6 animals were used in this study.

Colonization of mice with spore forming microbes. Fecal pellets were taken from WT mice and incubated in reduced PBS containing 3% chloroform (v/v) for 1 hour at 37° C. in an anaerobic chamber. A control tube containing reduced PBS and 3% chloroform was also incubated for 1 hour at 37° C. in an anaerobic chamber. After incubation, tubes were gently mixed and fecal material was allowed to settle for 10 seconds. Supernatant was transferred to a fresh tube and chloroform was removed by forcing CO2 into the tube. For spore-forming (SF) experiments in conventional conditions, mice within the SF cohort were orally gavaged with 1004 of spore forming fecal fraction, and mice within the CTRL cohort were orally gavaged with 1004 of PBS control that also had chloroform removed every third day. For spore-forming associations with germ-free animals, tubes containing gavage material were sterilized in the port of a germfree isolator for 1 hour before pulling them into the isolator for gavage. Breeder pairs were then orally gavaged with 100 μL of the spore-forming cocktail. Their offspring were sacrificed at 8 weeks of age for analysis of the small intestine and liver.

T cell transfer into T-Myd88^{-/-} mice. T-Myd88^{-/-} mice were sublethally irradiated with 500 rads the day before T cell transfer. Spleens from WT (CD4-cre⁺) and BCL6KO (Bcl6^{LoxP/LoxP} CD4-cre⁺) mice were used, and T cells were isolated using the CD4⁺ T Cell Isolation Kit (Miltenyi). T-Myd88^{-/-} were retro-orbitally injected with 5×10⁶ of either the WT or Bcl6^{-/-} MACS-enriched T cells and weighed weekly for 5 weeks.

Diet treatment. Animals housed within the SPF facility were fed a standard chow of irradiated 2920× (Envigo). Mice were fed a high fat diet of 45 kcal % fat DIO mouse feed (Research Diets) or a diet of 10 kcal % fat DIO mouse feed (Research Diets) as a control during HFD experiments. Mice were also fed a custom diet containing irradiated standard 2020 chow containing 1% 5-ASA (Envigo) or a control diet lacking the 5-ASA (Envigo) during 5-ASA inflammation experiments.

Antibiotics treatment. WT and T-Myd88^{-/-} mice were maintained on 0.5 mg/mL of ampicillin (Fisher Scientific), neomycin (Fisher Scientific), erythromycin (Fisher Scientific), and gentamicin (GoldBio) within their drinking water for 14 weeks while being fed a HFD in order to determine the relative contribution of the microbiota to the weight gain phenotype. TCRb^{-/-} and Rag1^{-/-} mice were placed on 0.5 mg/mL of ampicillin (Fisher Scientific), neomycin (Fisher Scientific), erythromycin (Fisher Scientific), and gentamicin (GoldBio) within their drinking water for 1 week to reduce the endogenous microbiota before being recolonized by fecal transfers.

T cell shaping of the microbiota within Tcrb^{-/-} mice. Three separate cages of four Tcrb^{-/-} mice were placed on an antibiotic cocktail within their drinking water for one week. Antibiotics was removed for 24 hours before any further treatment. One fecal pellet from a WT donor and one fecal pellet from a T-Myd88^{-/-} donor were mashed in reduced PBS containing 0.1% cysteine and immediately orally gavaged into the Tcrb^{-/-} mice. This oral gavage was repeated every other day for one week. Forty-eight hours following the final gavage, mice were placed into individually housed cages and retro-orbitally injected with 5×10⁶ CD4⁺ MACS-enriched WT or T-Myd88^{-/-} cells. This was labeled as DO.

Glucose tolerance test. Mice were fasted for 6 hours prior to being challenged with glucose. Fasting levels of glucose were detected using a Contour Glucose Meter (Bayer) and Contour Glucose Strips (Bayer). One milligram of glucose per gram of body weight was injected intraperitoneally into animals at timepoint zero. Blood levels of glucose were measured at 5-, 15-, 30-, 60-, and 120-min time points using the glucose meter and strips.

Insulin ELISA. Serum was collected from 6-hour fasted mice, and insulin was measured using a mouse insulin ELISA kit (Crystal Chem). Serum samples were run in duplicate according to the manufacturer instructions.

Insulin resistance test. Mice were fasted for 6 hours prior to being challenged with glucose. Fasting levels of glucose were detected using a Contour Glucose Meter (Bayer) and Contour Glucose Strips (Bayer). Insulin (0.75U/kg of body weight) was injected intraperitoneally into animals at timepoint zero. Blood levels of glucose were measured at 5-, 10-, 15-, 20-, 25-, 30-, 40-, and 60-min time points using the glucose meter and strips. Animals were removed from the experiment following an 150 μL i.p. injection of 25% glucose if blood glucose levels dropped to 30 mg/dL.

In vitro experiments using mouse intestinal epithelial cells (MODE-K cells). Mouse intestinal epithelial cells were maintained in Dulbecco's modified Eagle's medium (DMEM), with L-glutamine and sodium pyruvate. DMEM was supplemented with 10% FBS, 1% (v/v) glutamine, penicillin-streptomycin, and 1% HEPES. To determine if bacteria regulated gene expression, a confluent monolayer of cells was incubated with (1:1) DMEM without penicillin-streptomycin:CFS collected from either cultured Clostridia consortia or Desulfovibrio species for 4 hours. Media was then aspirated and cells were placed in 600 tL RiboZol (VWR) for later analysis.

RNA isolation from small intestine, cell culture and liver tissue for qPCR and RNA-seq. Tissue sections 0.5 cm in length or 1×10⁵ cells were stored at −70° C. in 700 tL of RiboZol (VWR). RNA was isolated using the Direct-zol RNA MiniPrep Kit (Zymoresearch). cDNA was synthesized using qScript cDNA synthesis kit (Quanta Biosciences). qPCR was conducted using LightCycler 480 SYBR Green I Master (Roche). qPCR experiments were conducted on a Lightcycler LC480 instrument (Roche). For liver RNA sequencing, RNA was prepped following QC via an Illumina TruSeq Stranded RNA Sample Prep with RiboZero treatment (human, mouse, rat, etc.) and analyzed using Illumina HiSeq Sequencing.

Quantification of fecal immunoglobulins. To quantify luminal IgA, fecal pellets were collected in 1.5 mL microcentrifuge tubes and weighed. Luminal contents were resuspended in 10 tL of sterile 1× HBSS per milligram of fecal weight and spun at 100×g for 5 minutes to remove course material. Supernatants were then placed in a new 1.5 mL microcentrifuge tube and spun at 8000×g for 5 min to pellet bacteria.

Supernatants (containing IgA) were then placed in a new 1.5 mL microcentrifuge tube and used as samples (1/10 and 1/100 (v/v) dilutions) for an IgA-specific ELISA kit (eBioscience; performed according to manufacturer instructions). Absorbance was read at 450 nm andconcentrations of IgA were calculated using a standard curve. Concentrations were normalized to fecal weight.

Bacterial pellets were resuspended in 500 tL of sterile PBS and washed twice by spinning at 8000×g for 5 min. The washed bacterial pellet was then resuspended in 10 tL of sterile PBS per mg of feces. Five microliters of each sample was plated on to a 96-well round-bottom plate. Bacteria were blocked for 15 min at room temperature with 100 tL of sterile HBSS containing 10% (v/v) FBS. Without washing cells, 100 tL of anti-IgA (ebioscience clone mA-6E1 PE), anti-IgG1 (Santacruz CruzFluor555), or anti-IgG3 (Santacruz CruzFluor555) diluted at 1:500 in sterile HBSS containing 10% (v/v) FBS was added to the wells. Wells were incubated at 4° C. for 30 min. The plate was washed twice by spinning at 2500× g for 5 min before removing the supernatant and resuspending cells in sterile HBSS. After final wash, bacterial wells were resuspended in 250 tL of HBSS containing 5 tL of 1× SYBR green stain (Invitrogen cat #S7563). Wells were incubated for 20 min at 4° C. before immediate enumeration on a flow cytometer. Rag1^{-/-} fecal pellets were included in all experiments as negative controls.

Growth of Desulfovibrio desulfuricans ATCC 27774 and Desulfovibrio piger ATCC 29098. The bacterial species Desulfovibrio desulfuricans was purchased from ATCC (#27774). The bacterial species Desulfovibrio piger was purchased from ATCC (#29098). The vial was handled and opened per ATCC instructions for anaerobic bacteria and cells were grown in Desulfovibrio media described previously (F. E. Rey et al., Proc Natl Acad Sci USA 110, 13582-13587 (2013)). Media was composed of NH4Cl (1 g/L) (Fisher Chemical), Na2SO4 (2 g/L) (Fisher Chemical), Na2S2O3.5H2O (1 g/L) (Sigma), MgSO4.7H2O (1 g/L) (Fisher Chemical), CaCl2.2H2O (0.1 g/L) (Fisher Chemical), KH2PO4 (0.5 g/L) (Fisher Bioreagents), Yeast Extract (1 g/L) (Amresco), Resazurin (0.5 mL/L) (Sigma), cysteine (0.6 g/L) (Sigma), DTT (0.6 g/L) (Sigma), NaHCO₃(1 g/L) (Fisher Chemical), pyruvic acid (3 g/L) (Acros Organics), malic acid (3 g/L) (Acros Organics), ATCC Trace Mineral Mix (10 mL/L), ATCC Vitamin Mix (10 mL/L) and adjusted to pH of 7.2. Bacteria were grown for 48 hrs in an anaerobic chamber (Coy Labs) and stored in growth media containing 25% glycerol at 70° C. 2.5×10⁸ bacterial CFUs were added to 250 μL of drinking water of mice for 1 week.

Isolation and 16S sequencing of fecal, ileal and IgA bound microbial DNA. Animals were sacrificed and their entire lower digestive tract (from duodenum to rectum) was removed and longitudinally sectioned. One fecal pellet and luminal content from lower 10 cm of small intestine were collected from each animal to characterize the fecal and ileal microbiota communities, respectively. Fecal and ileal samples were immediately frozen at −70° C. in 2 mL screw cap tubes containing ˜250 mg of 0.15 mm garnet beads (MoBio, cat#13122-500). DNA was extracted using the Power Fecal DNA Isolation Kit (MoBio), according to manufacturer instructions. IgA-bound and -unbound bacteria from T cell transfer experiments were isolated from cecal contents and frozen at −70° C. before processing. IgA bound bacteria separation, 16S rDNA amplification, sequencing and sequence processing was done (J. L. Kubinak et al., Cell Host Microbe 17, 153-163 (2015)), using paired-end 300 cycle MiSeq reads. The IgA index was calculated (A. L. Kau et al., Sci Transl Med 7, 276ra224 (2015)).

Metatranscriptomics. Fecal pellets or lumenal ileal contents were placed directly into Trizol and stored at −20° C. until RNA extraction. Total RNA was extracted from samples using Direct-zol (Zymo Research, #R2052), then prepared for Illumina sequencing by the University of Utah high-throughput genomics core facility using the Ribo-Zero Gold rRNA (epidemiology) removal kit (Illumina, #MRZE724). Illumina libraries were multiplexed and sequenced on a HiSeq 2500 with single-end 50 cycle sequencing. The humann2 (v 0.9.9) analysis framework was used for the subsequent sequencing processing and data analysis (S. Abubucker et al., PLoS Comput Biol 8, e1002358 (2012)). First, using the knead data script implemented in Humann2, raw sequences were quality trimmed and filtered using Trimmomatic (A. M. Bolger, M. Lohse, B. Usadel, Bioinformatics 30, 2114-2120 (2014)), then filtered to remove host reads against the Mus musculus genome build GRCm38 using bowtie2 (B. Langmead, S. L. Salzberg, Nat Methods 9, 357-359 (2012). No significant difference in quality-filtered reads was observed among genotypes, although across the samples many more reads from ileal samples mapped to the mouse genome, providing less bacterial transcript coverage. Then, to improve mapping of these short reads, mapping of the quality-filtered reads was restricted to a custom database of mouse isolated bacterial reference genomes with UniRef90 gene annotations. This custom database consisted of 53 organisms isolated and sequenced recently as part of the mouse intestinal bacterial collection (miBC) (I. Lagkouvardos et al., Nat Microbiol 1, 16131 (2016)), as well as nine reference genomes included in humman2's chocophlan database, representing species that were detected in 16S sequencing but that were not included in the miBC collection already. These nine genomes were: Bifidobacterium pseudolongum, Bifidobacterium animalis, Bifidobacterium longum, Bacteroides fragilis, Mucispirillum schaedleri, Lactobacillus reuteri, Clostridium perfringens, Desulfovibrio desulfuricans, and Candidatus Arthromitus. To create the custom database with Uniref90 annotations, the amino acid sequences from the miBC genomes were aligned to the Uniref90 database using the Diamond aligner (B. Buchfink, C. Xie, D. H. Huson, Nat Methods 12, 59-60 (2015)) and requiring 50% query coverage and 90% identity. Then, these uniref90-annotated miBC amino acid sequences were used to annotate each corresponding gene's nucleotide sequences and combined with the nine genomes already annotated to create the custom nucleotide mapping reference containing mouse-specific bacterial genomes. For mapping filtered sequence reads to the custom reference using Humann2, the nucleotide alignments (no translated alignments) were used due to the short read length. The counts of aligned reads per kilobase for uniref90 gene families output from humann2 were then normalized to counts per million (within a sample), or regrouped to Gene Ontology (GO) terms then normalized, for the subsequent analyses.

Metabolic Phenotyping. Total body fat composition was measured on an NMR Bruker Minispec. CLAMS Metabolic Cages were used to measure indirect calorimetry. Energy expenditure (EE) was calculated using the following formulas. Calorific Value (CV)=3.815+(1.232*RER). EE=CV*V02.

Liver and adipose tissue microscopy. Liver and adipose tissue were fixed in formalin, embedded in wax, and Hematoxylin and eosin stained. Microscopy images were collected using an EVOS core XL imaging system from Thermofisher.

Serum and cecal content metabolomics (Excluding SCFA measurements). Sample Extraction and Preparation. Cecal contents were stored at −70° C. prior to analysis. Five mililiters of 75% ethanol solution containing internal standards (1 tg of d4-succinic acid and 5 tg of labeled amino acids (¹³C, ¹⁵N-labeled) mixture per sample) was added to each sample. Samples were vigorously vortexed and then incubated in boiling water for 10 min. Cooled samples were spun down at 5,000× g for 5 min. Supernatants were transferred to fresh tubes and then speed-vacuumed overnight to dry.

GC-MS analysis. The GC-MS analysis was performed with a Waters GCT Premier mass spectrometer fitted with an Agilent 6890 gas chromatograph and a Gerstel MPS2 autosampler. Dried samples were suspended in 40 tL of a 40 mg/mL 0-methoxylamine hydrochloride (MOX) in pyridine and incubated for 1 hour at 30° C. To autosampler vials was added 25 tL of this solution. Forty microliters 40 tL of N-methyl-N-trimethylsilyltrifluoracetamide (MSTFA) was added automatically via the autosampler and incubated for 60 minutes at 37° C. with shaking. After incubation 3 tL of a fatty acid methyl ester standard (FAMES) solution was added via the autosampler then 1 μL of the prepared sample was injected to the gas chromatograph inlet in the split mode with the inlet temperature held at 250° C. A 10:1 split ratio was used for analysis. The gas chromatograph had an initial temperature of 95° C. for 1 minute followed by a 40° C./min ramp to 110° C. and a hold time of 2 minutes. This was followed by a second 5° C./min ramp to 250° C., a third ramp to 350° C., then a final hold time of 3 minutes. A 30-m Phenomex ZB5-5 MSi column with a 5-m long guard column was employed for chromatographic separation. Helium was used as the carrier gas at 1 mL/min. Due to the high amounts of several metabolites the samples were analyzed once more at a tenfold dilution.

Analysis of GC-MS data. Data were collected using MassLynx 4.1 software (Waters). Metabolites were identified and their peak area was recorded using QuanLynx. This data was transferred to an Excel spread sheet (Microsoft, Redmond Wash.). Metabolite identity was established using a combination of an in house metabolite library developed using pure purchased standards and the commercially available NIST library. Not all metabolites are observed using GC-MS. This was due to several reasons. For example, some metabolites were present at very low concentrations. Second, metabolites may not be amenable to GC-MS due to either being too large to volatilize, are a quaternary amine such as carnitine, or just do not ionize well. Metabolites that do not ionize well include oxaloacetate, histidine, and arginine. Cysteine is observed depending upon cellular conditions, often forms disulfide bonds with proteins, and is at a low intracellular concentration.

Short chain fatty acid detection of cecal contents. Sample extraction and preparation. Samples were removed from freezer and allowed to thaw at RT for 5 min. To these samples were added 400 tL of dd-H2O, 10 tL of 5-sulfosalicylic acid (1 mg/tL), and 2 tL of internal standard (1 M pivalic acid). Samples were vortexed for 30 sec and rested on ice for 30 min. Samples were then centrifuged at 2000×g for 10 min at 4° C. The supernatants were then transferred to glass vials with PTFE lined caps containing 10 tL of concentrated HCl. Next, 3 mL of ether was added and the samples were vortexed for 30 sec, then centrifuged at 1,200×g for 10 min at 4° C. The supernatants were then transferred to new glass vials with PTFE-lined caps and derivatized with 50 tL of N-Methyl-N-(tert-butyldimethylsilyl) trifluoroacetamide, tertbutyldimetheylchlorosilane (MTBSTFA; Thermo Scientific). Samples were vortexed and placed in a 60° C. sand bath for 30 min. Samples were allowed to cool to RT and partially evaporated under a gentle stream of nitrogen to a volume of approximately 250 tL and transferred to glass GC-MS vials.

GC-MS analyses. GC/MS analyses were conducted on an Agilent 6890 gas chromatograph coupled to an Agilent 5793 mass spectrometer and an Agilent 7683 (Santa Clara, Calif., USA) auto-injector equipped with a DB-1 column (15 m×0.25-mm internal diameter×0.25-μm film thickness; J&W Scientific, Folsom, Calif., USA). Helium carrier gas was used with a flow rate of 1.0 mL/min. Split ratio 10:1 with injections of 1-4 samples were made into an inlet held at 250° C. The GC oven ramp used was 40° C. (hold 1 min); ramp at 5° C./min to 70° C. (hold 3 min); ramp at 20° C./min to 160° C. (hold 0 min); ramp at 40° C./min to 330° C. (hold 6 min). Data were acquired in scan mode with a mass range of 44-200 m/z, targets were quantitated using m/z 117.0 for acetic acid, m/z 131.0 for butanoic acid, m/z 145.0 for propanoic acid and m/z 159.0 pivalic acid.

TABLE 2
Primers.

| | | SEQ ID |
|---|---|---|
| Primers | Sequence | NO: |
| L32 | For: 5′-AAGCGAAACTGGCGGAAAC-3′ | 33 |
| | Rev: 5′-TAACCGATGTTGGGCATCAG-3′ | 34 |
| CD36 | For: 5′-TCCTCTGACATTTGCAGGTCTATC-3′ | 35 |
| | Rev: 5′-AAAGGCATTGGCTGGAAGAA-3′ | 36 |
| FASN | For: 5′-GGAGGTGGTGATAGCCGGTAT-3′ | 37 |
| | Rev: 5′-TGGGTAATCCATAGAGCCCAG-3′ | 38 |

### Example 2: Four Clostridia Strains Rescue Obesity, Insulin Resistance and Inflammatory Bowel Disease

A formulation of 4 Clostridia members reduces adiposity in mice. Various in vitro techniques have been employed to narrow down the specific members of the community that contribute to reduced adiposity. These experiments have led to the testing of a combination of 4 specific members of this community. A refined 4 member community that contains Clostridia anaerovorax, Lachnospiraceae spps, Clostridium XIVa, and Clostridium IV, (also referred to herein as refined Clostridia consortia-rCC-4). These 4 strains were used to colonize mice and compare fat accumulation to the more complex Clostridia consortia (called SF in the figure). Interestingly, these 4 strains were sufficient to reduce adiposity (rCC-M) to the same degree as the complex microbial community (FIG. 18).

Replacement of Clostridia rescues obesity, insulin resistance and inflammatory bowel disease. Through a series of experiments, a significant reduction in the class Clostridia was identified in the obesogenic animals. Therefore, it was tested whether reintroduction of these lean-associated microbes could protect against obesity. Since Clostridia are known spore-formers, the feces was treated with chloroform to enrich for these microbes. The animals were placed on a high fat diet (HFD), which has been shown to speed up weight gain in this model and better mimics a westernized lifestyle. Treatment of obesity prone T-MyD88^{-/-} animals with a cocktail of spore-forming bacteria significantly reduced weight gain and fat accumulation (FIGS. 19A-B). At the end of just three months, T-MyD88^{-/-} mice treated with Clostridia had a lower body fat percentage and a reduced VAT mass when compared to untreated T-MyD88−/−(FIG. 19C). Clostridia treatment also decreases blood glucose levels and reduces insulin resistance (FIGS. 19D and 19E). Importantly, improvement of these metabolic parameters by Clostridia treatment was also seen in WT animals placed on a HFD (FIGS. 19D and 19E; WT), supporting that improvement of MetS by Clostridia will be relevant in several different models of MetS and TIID.

While a relationship between IBD and diabetes has been controversial, there are several studies that now support this connection. In a cross-sectional study with 12,601 patients with IBD, diabetes was the third most common co-morbidity. Another more recent cross-sectional study from 47,325 patients in Denmark showed that diabetes was significantly associated with IBD (both UC and CD). In a pediatric cohort of 1200 IBD patients, the prevalence of diabetes was also higher in UC patients than controls. A more recent study, in 2019, using 8070 patients with IBD and 40,030 healthy controls demonstrated that the incidence of diabetes was significantly higher in individuals with IBD even when controlling for steroid use. Finally, a nation-wide population based cohort of 6,028,844 individuals with a diagnosis of IBD versus without IBD from 1977 to 2014, revealed an increased incidence of diabetes in individuals with IBD, especially between the years 2003-2014. In addition to glucose homeostasis, individuals with IBD have also been reported to have changes in lipid metabolism, supporting a link between metabolic disease and IBD. Commonalities to both of these diseases include chronic inflammation and perturbations to the microbiota, however, the mechanisms underlying the connection between these ailments are unknown.

There have now been exhaustive studies that have analyzed the microbiota composition in individuals with diabetes and IBD in comparison to healthy controls, and several similarities have emerged. Decreased diversity of the microbiota, with a specific depletion of members of the Clostridiales, Ruminococcaceae and Lachnospiraceae is reported in individuals with diabetes, obesity and IBD. While the microbiota composition at the phylogenetic level is generally variable between individuals, the functional capacity of the microbiota is quite stable. Thus, metagenomic studies may provide more insight into the contribution of the microbiota to disease. Metagenomic studies in both Type II diabetes and IBD have revealed a decrease in short chain fatty acid (SCFAs) production, which is consistent with the decrease in members of the Clostridia. In addition to the loss of specific subsets of organisms, there are similar pathobionts that have been identified among these disorders. A study that looked at over 350 individuals with TIID and compared the composition of the microbiota with that of healthy individuals identified that the most significant shifts associated with TIID was an enrichment in the sulfate reducing organisms, Desulfovibrio. Another independent study identified Desulfovibrio overgrowth in an immune-compromised individual that also had TIID. In IBD, a number of studies note an increase in Desulfovibrio. Mesalamine, which is a common treatment for IBD, inhibits fecal sulfide, however, in patients that have not taken this drug, possess higher levels of sulfide. Metagenomic studies confirm the phylogenetic studies in IBD and Type II diabetes, and have demonstrated that genes involved in the metabolism of the sulfur containing amino acid, cysteine, are increased in individuals with disease. Thus, similar shifts in the composition of the microbiota are identified in individuals with IBD and diabetes, suggesting that these commonalities may underlie the development of these diseases.

Based on the connection between diabetes and IBD, it was tested whether these bacteria could rescue or be protective from a mouse model of IBD. A chronic model of dextran sodium sulfate (DSS) colitis was used whereby DSS is provided in the drinking water for 5 days followed by 10 days of regular water and repeated two additional cycles. Clostridia or PBS was orally gavaged every other day and histology was performed. Indeed, animals treated with Clostridia were significantly protected from the development of colitis as determined by increased colon length and reduced histopathology scores (FIGS. 19F, 19G). Thus, Clostridia can prevent the development of metabolic syndrome (MetS) and IBD.

What is claimed is:

1. A composition comprising a supernatant from a Clostridia consortium.

2. The composition of claim 1, wherein the Clostridia consortium comprises two or more strains of bacterium, wherein the two or more strains of bacterium are Clostridia anaerovorax, Clostridium XIVa, Clostridium IV and Lachnospiraceae spps.

3. The composition of claim 1, wherein the composition is capable of suppressing expression of lipid absorption genes within intestinal epithelia in a subject, and wherein the Clostridia consortium comprises two or more of Clostridia anaerovorax, Clostridium XIVa, Clostridium IV and Lachnospiraceae spps.

4. The composition of claim 1, wherein the composition is capable of inhibiting lipid absorption in a subject's small intestine, and wherein the Clostridia consortium comprises two or more of Clostridia anaerovorax, Clostridium XIVa, Clostridium IV and Lachnospiraceae spps.

5. The composition of claim 1, wherein the composition is capable of reducing weight gain in a subject, and wherein the Clostridia consortium comprises two or more of Clostridia anaerovorax, Clostridium XIVa, Clostridium IV and Lachnospiraceae spps.

6. The composition of claim 1, wherein the composition is capable of downregulating CD36 in a subject's liver, and wherein the Clostridia consortium comprises two or more of Clostridia anaerovorax, Clostridium XIVa, Clostridium IV and Lachnospiraceae spps.

7. The composition of claim 1, wherein the composition is capable of reducing adiposity in a subject, and wherein the Clostridia consortium comprises two or more of Clostridia anaerovorax, Clostridium XIVa, Clostridium IV and Lachnospiraceae spps.

8. The composition of claim 1, wherein the composition is capable of lowering body fat percentage and/or reducing visceral adipose tissue (VAT) mass in a subject, and wherein the Clostridia consortium comprises two or more of Clostridia anaerovorax, Clostridium XIVa, Clostridium IV and Lachnospiraceae spps.

9. The composition of claim 1, wherein the composition is capable of decreasing blood glucose levels and/or reducing insulin resistance in a subject, and wherein the Clostridia consortium comprises two or more of Clostridia anaerovorax, Clostridium XIVa, Clostridium IV and Lachnospiraceae spps.

10. A composition comprising a Clostridium consortium.

11. The composition of claim 10, wherein the composition is capable of suppressing expression of lipid absorption genes within intestinal epithelia in a subject.

12. The composition of claim 10 or 11, wherein the Clostridium consortium comprises Clostridia anaerovorax genera, Clostridium XIVa, and Clostridium IV, and Lachnospiraceae spps.

13. The composition of claim 1, further comprising one or more bacterial strains selected from Table 1.

14. The composition of any of the preceding claims, further comprising a pharmaceutically acceptable carrier.

15. The composition of any of the preceding claims wherein the composition is frozen.

16. The composition of any of the preceding claims, wherein the composition is a solid.

17. The composition of any of the preceding claims, wherein the composition comprises at least 1×10⁻⁵ cells of each Clostridia strain.

18. The composition of any of the preceding claims, wherein a single dosage of the composition comprises between 1×10⁻⁵ and 1×10⁻¹⁰ cells of each Clostridia strain.

19. The composition of preceding claims, wherein the composition is capable of replacing microbiota of a subject with a disease or disorder associated with an imbalanced microbiota.

20. The composition of claim 19, wherein the imbalanced microbiota is an increase in Desulfovibrio and a decrease of Clostridia.

21. The composition of claim 19, wherein the imbalanced microbiota is a decrease of Clostridia and no expansion Desulfovibrio.

22. The composition of claim 19, wherein the disease or disorder is obesity, metabolic syndrome, insulin deficiency, insulin-resistance related disorders, glucose intolerance, diabetes, or an inflammatory bowel disease.

23. The composition of any of the preceding claims, wherein the composition is administered in a form selected from the group consisting of powder, granules, a ready-to-use beverage, food bar, an extruded form, capsules, gel caps, and dispersible tablets.

24. A consortium of bacteria comprising two or more of Clostridia anaerovorax, Clostridium XIVa, Clostridium IV, and Lachnospiraceae spps, wherein the consortium suppresses expression of lipid adsorption genes within intestinal epithelia in a subject compared to a subject where the consortium has not been administered.

25. A method of altering relative abundance of microbiota in a subject, the method comprising administering to the subject an effective dose of the composition of any of the preceding claims, thereby altering the relative abundance of microbiota in the subject.

26. The method of claim 25, wherein the relative abundance of Clostridia bacteria is increased or replaced.

27. The method of claim 25, wherein the relative abundance of Clostridia is increased in the subject by at least about 5%.

28. The method of claim 25, further comprising administering a second therapeutic agent to the subject.

29. A method of treating a subject with obesity, the method comprising administering to the subject the composition of any of claims 1-24, wherein the relative abundance of Clostridia is increased in the subject compared to the relative abundance prior to administration.

30. A method of treating a subject with metabolic syndrome, the method comprising administering to the subject the composition of any of claims 1-24, wherein the relative abundance of Clostridia is increased in the subject compared to the relative abundance prior to administration.

31. A method of treating a subject with irritable bowel disease, the method comprising administering to the subject the composition of any of claims 1-24, wherein the relative abundance of Clostridia is increased in the subject compared to the relative abundance prior to administration.

32. A method of reducing weight gain in a subject, the method comprising administering to the subject the composition of any of claims 1-24, wherein the relative abundance of Clostridia is increased in the subject compared to the relative abundance prior to administration.

33. A method of inhibiting lipid absorption in a subject's small intestine, the method comprising administering to the subject the composition of any of claims 1-24, wherein the relative abundance of Clostridia is increased in the subject compared to the relative abundance prior to administration.

34. A method of downregulating CD36 in a subject's liver, the method comprising administering to the subject the composition of any of claims 1-24, wherein the relative abundance of Clostridia is increased in the subject compared to the relative abundance prior to administration.

35. The method of any of the preceding claims, wherein the subject has been identified as being in need of the treatment.

36. The method of any of claim 25-28, 32, 33 or 34, wherein the subject has obesity, metabolic syndrome, insulin deficiency, insulin-resistance related disorders, glucose intolerance, diabetes, or an inflammatory bowel disease.

37. The method of claim 36, wherein the inflammatory bowel disease is Crohn's disease or ulcerative colitis.

38. The method of claim 36, wherein the insulin-resistance related disorder is diabetes, hypertension, dyslipidemia, or cardiovascular disease.

39. The method of any of the preceding claims, wherein the step of administering the composition comprises delivering the composition to at least a stomach, a small intestine, or a large intestine of the subject.

40. The method of any of the preceding claims, wherein the composition is administered orally.

41. The method of any of the preceding claims, wherein the relative abundance of at least one of species of Clostridia is increased by 5%.

42. The method of any of the preceding claims, wherein the subject is a human.

43. The composition or method of any of the preceding claims, wherein the cells of the consortia are active.

44. The method of any of preceding claims, wherein the composition is for replacing microbiota of a subject with a disease or disorder associated with an imbalanced microbiota.
